(19) United States
(12) Patent Application Publication
(10) Pub. No.: US 2026/0048066 A1
(43) Pub. Date: Feb. 19, 2026

(54) TREATMENT OF NEURODEGENERATIVE DISORDERS UTILISING METHYLTHIONINIUM (MT)-CONTAINING COMPOUNDS

(71) Applicant: WISTA LABORATORIES LTD, Singapore (SG)

(72) Inventors: Claude Michel WISCHIK, Aberdeen (GB); John Mervyn David STOREY, Old Aberdeen (GB); Björn Olaf SCHELTER, Aberdeen (GB)

(73) Assignee: WISTA LABORATORIES LTD, Singapore (SG)

(21) Appl. No.: 18/869,709

(22) PCT Filed: May 30, 2023
(86) PCT No.: PCT/EP2023/064369

§ 371 Date: Nov. 26, 2024

(30) Foreign Application Priority Data
May 31, 2022 (GB) 2208038.6
Oct. 5, 2022 (GB) 2214646.8

(51) Int. Cl. A61K 31/5415; A61P 25/28

(52) U.S. Cl. CPC A61K 31/5415; A61P 25/28

(57) ABSTRACT

The invention provides methods of treatment of a neurodegenerative disease in a subject, which methods comprise orally administering to said subject a methylthioninium (MT) containing compound, wherein said administration is at a dosage frequency of less than once daily; and wherein said administration provides an amount of MT to the subject that corresponds to an average of between 0.05 mg and 30 mg MT per day, preferably between 0.1 mg and 20 mg MT per day. The invention further provides related compositions and kits of parts for use in the method.

### CROSS-REFERENCE TO RELATED APPLICATIONS

This patent application claims the benefit of priority of GB 2208038.6 filed 31 May 2022 and GB 2214646.8 filed 5 Oct. 2022, which are herein incorporated in their entirety.

### FIELD OF THE INVENTION

The present invention relates generally to methods and materials for use in the treatment or prophylaxis of diseases of protein aggregation, for example cognitive disorders, using diaminophenothiazines.

### BACKGROUND

Aberrant protein aggregation is believed to be a proximal cause of numerous disease states, which may be manifested as neurodegeneration, clinical dementia, and other pathological symptoms.

In general, the aberrant protein aggregation is that which arises from an induced conformational polymerisation interaction, i.e., one in which a conformational change of the protein, or in a fragment thereof, gives rise to templated binding and aggregation of further (precursor) protein molecules in a self-propagating manner.

Once nucleation is initiated, an aggregation cascade may ensue which involves the induced conformational polymerisation of further protein molecules, leading to the formation of toxic product fragments in aggregates which are substantially resistant to further proteolysis.

For example certain conditions of dementia may be characterised by a progressive accumulation of intracellular and/or extracellular deposits of proteinaceous structures such as β-amyloid plaques and neurofibrillary tangles (NFTs) in the brains of affected patients. The appearance of these lesions largely correlates with pathological neurofibrillary degeneration and brain atrophy, as well as with cognitive impairment (see, e.g., Mukaetova-Ladinska, E. B. et al., 2000, Am. J. Pathol., Vol. 157, No. 2, pp. 623-636).

Current approved treatments for Alzheimer's disease include acetylcholinesterase inhibitors (AChEls) and the N-methyl-D-aspartate receptor antagonist memantine. These are symptomatic and do not address the underlying disease pathology. Therapies targeting the amyloid pathology have so far proved unsuccessful in late-stage clinical trials (Geerts et al., 2013; Mullane and Williams, 2013). According to a recent Lancet Neurology Commission, “an effective treatment for AD is perhaps the greatest unmet medical need facing modern medicine”, (Winblad et al., 2016) not least because the global economic cost of dementia is estimated to be $818 billion, or 0.65% of global gross domestic product (Alzheimer's Disease International, 2015).

NFTs (the pathology discovered by Alois Alzheimer, (Alzheimer, 1907)) are made up of paired helical filaments (PHFs), composed predominantly of a 12-kDa repeat-domain fragment of the microtubule-associated protein tau (Wischik et al., 1985; Wischik et al., 1988a,b). Numerous studies have confirmed a quantitative link for the spread of neurofibrillary tangle pathology and the quantity of aggregated tau with both the extent of clinical dementia and functional molecular imaging deficits in Alzheimer's disease (Arriagada et al., 1992; Brier et al., 2016; Giannakopoulos et al., 2003; Josephs et al., 2003; Maruyama et al., 2013). Since pathological aggregation of tau protein begins at least 20 years prior to any of the clinical manifestations, (Braak and del Tredici, 2013) targeting this pathology offers a rational approach to both treatment and prevention of AD and related tau aggregation disorders (Huang and Mucke, 2012; Wischik et al., 2014; Wischik et al., 2010).

The tau fragment originally identified as an intrinsic structural constituent of the PHF core has prion-like properties in vitro in that it captures normal tau protein with very high affinity (Lai et al., 2016) and converts it to a proteolytically stable replicate of itself (Wischik et al., 1996; Harrington et al., 2015) in a process which is self-propagating and autocatalytic. Phosphorylation is inhibitory to aggregation (Lai et al., 2016) and is unlikely to drive the cascade (Mukaetova-Ladinska et al., 2000; Schneider et al., 1999; Wischik et al., 1995). Direct inhibition of tau aggregation represents a plausible point for therapeutic intervention.

Methylthioninium (MT) acts as a tau aggregation inhibitor (TAI) in vitro, (Wischik et al., 1996; Harrington et al., 2015), dissolves PHFs from Alzheimer's disease brain tissue, (Wischik et al., 1996) and reduces tau pathology and associated behavioural deficits in transgenic mouse tau models at brain concentrations consistent with human oral dosing. (Melis et al., 2015; Baddeley et al., 2015) MT has also been shown to inhibit other disease-associated protein aggregation (see e.g. WO2007/110629).

MT is a redox molecule and, depending on environmental conditions (e.g., pH, oxygen, reducing agents), exists in equilibrium between a reduced [leucomethylthioninium (LMT)] and oxidized form (MT⁺).

Leucomethylthioninium (LMT) is the active moiety in compounds such as leucomethylthioninium mesylate (LMTM). Leucomethylthioninium (LMT) may also be referred to as hydromethylthionine (HMT). For the avoidance of doubt, these two terms are synonymous and may be used interchangeably herein. Similarly. the terms LMTM (leucomethylthioninium mesylate) and HMTM (hydromethylthionine mesylate) may be used interchangeably herein.

WO96/30766 describes such MT-containing compounds for use in the treatment and prophylaxis of various diseases, including AD and Lewy Body Disease. One example compound was methylthioninium chloride (“MTC”) commonly known as methylene blue, which is the chloride salt of the oxidized form of methylthioninium (MT) i.e. MT⁺.

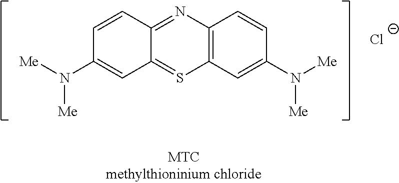

WO96/30766 describes, in the case of oral administration, a daily dosage of about 50 mg to about 700 mg, preferably about 150 mg to about 300 mg, divided in preferably 1-3 unit doses.

WO2007/110630 discloses certain specific diaminophenothiazine compounds related to MTC, including (so-called) ETC, DEMTC, DMETC, DEETC, MTZ, ETZ, MTI, MTILHI, ETI, ETLHI, MTN, and ETN, which are useful as drugs, for example in the treatment of Alzheimer's disease.

WO2007/110630 describes dosage units comprising 20 to 300 mg of 3,7-diaminophenothiazine (DAPTZ) compounds described therein e.g. 30 to 200 mg, for example 30 mg, 60 mg, 100 mg, 150 mg, 200 mg. A suitable dose of the DAPTZ compound is suggested in the range of about 100 ng to about 25 mg (more typically about 1 μg to about 10 mg) per kilogram body weight of the subject per day e.g. 100 mg, 3 times daily, 150 mg, 2 times daily, 200 mg, 2 times daily. A dosage of 50 mg 3 or 4 times daily is also discussed.

A preliminary pharmacokinetic model for methylene blue, based on studies of urinary excretion data sets in humans, dogs and rats, was proposed by DiSanto and Wagner, J Pharm Sci 1972, 61:1086-1090 and 1972, 61:1090-1094 and Moody et al., Biol Psych 1989, 26:847-858.

Peter et al. (2000) Eur J Clin Pharmacol 56:247-250 provided a model which integrated blood level data, which contradicted the earlier data from DiSanto and Wagner as regards terminal elimination half-life.

May et al. (Am J Physiol Cell Physiol, 2004, Vol. 286, pp. C1390-C1398) showed that human erythrocytes sequentially reduce and take up MTC i.e. that MTC itself is not taken up by the cells but rather that it is the reduced from of MT that crosses the cell membrane. They also showed that the rate of uptake is enzyme dependent; and that both oxidised and reduced MT are concentrated in cells (reduced MT re-equilibrates once inside the cell to form oxidised MT).

Based on these and other disclosures, it is believed that orally administered MTC and similar drugs are taken up in the gut and enter the bloodstream, with unabsorbed drug percolates down the alimentary canal, to the distal gut. One important undesired side-effect is the effect of the unabsorbed drug in the distal gut, for example, sensitisation of the distal gut and/or antimicrobial effects of the unabsorbed drug on flora in the distal gut, both leading to diarrhoea.

MTC was tested clinically in a phase 2 study (Wischik et al., 2015). Although the minimum safe and effective dose was identified as 138 mg/day, a higher dose of 218 mg/day had limited efficacy due to absorption limitations, most likely due to the need for the MT⁺ to be reduced to the leuco-MT (LMT) form to permit efficient absorption by passive diffusion.

WO2009/044127 disclosed the results of a phase 2 clinical trial, which indicated that MTC had two systemic pharmacological actions: cognitive effects and haematological effects, but that these actions were separable. Specifically the cognitive effects did not show a monotonic dose-response relationship, whereas the haematological effects did. It was proposed that two distinct species were responsible for the two types of pharmacological activity: MTC absorbed as the uncharged Leuco-MT form being responsible for the beneficial cognitive activity, and MTC absorbed as an oxidised dimeric species being responsible for the oxidation of haemoglobin. WO2009/044127 described how dosage forms could be used to maximise the bioavailability of the therapeutically active (cognitively effective) species whether dosing with oxidised or leuco-DAPTZ compounds.

Since it is the reduced form of MT that is taken up by cells, it has been proposed to administer a reduced form to patients. This may also reduce reliance on the rate-limiting step of enzymatic reduction.

MTC, a phenothiazin-5-ium salt, may be considered to be an “oxidized form” in relation to the corresponding 10H-phenothiazine compound, N,N,N′,N′-tetramethyl-10H-phenothiazine-3,7-diamine, which may be considered to be a “reduced form”:

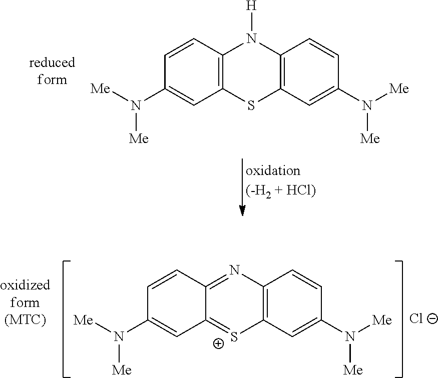

The “reduced form” (or “leuco form”) is known to be unstable and can be readily and rapidly oxidized to give the corresponding “oxidized” form.

WO 02/055720 discloses the use of reduced forms of certain diaminophenothiazines for the treatment of protein aggregating diseases, primarily tauopathies. Based on in vitro activity for the reduced forms of diaminophenothiazines therein, a suggested daily dosage was 3.2-3.5 mg/kg, and dosages of 20 mg t.d.s., 50 mg t.d.s. or 100 mg t.d.s., combined with 2×mg ratio of ascorbic acid in such a manner as to achieve more than 90% reduction prior to ingestion were also described.

WO2007/110627 disclosed certain 3,7-diamino-10H-phenothiazinium salts, effective as drugs or pro-drugs for the treatment of diseases including Alzheimer's disease. These compounds are also in the “reduced” or “leuco” form when considered in respect of MTC. These leucomethylthioninium compounds were referred to as “LMTX” salts, and included the following salts:

| | |
|---|---|
| 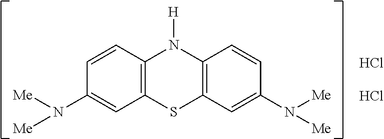 | N,N,N′,N′-tetramethyl-10H- phenothiazine-3,7-diaminium di(hydrochloride), (LMT.2HCl) |
| 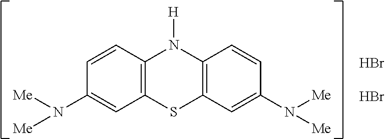 | N,N,N′,N′-tetramethyl-10H- phenothiazine-3,7-diaminium di(hydrobromide), (LMT.2HBr) |
| 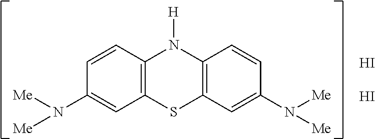 | N,N,N′,N′-tetramethyl-10H- phenothiazine-3,7-diaminium di(hydroiodide), (LMT.2HI) |

WO2012/107706 described other LMTX salts having superior properties to the LMTX salts listed above, including leuco-methylthioninium bis(hydromethanesulfonate) (LMTM; also known as HMTM, HMT, hydromethylthionine):

| | |
|---|---|
| 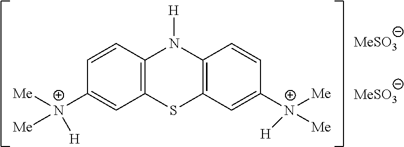 | N,N,N′,N′-tetramethyl-10H- phenothiazine-3,7- diaminium bis(hydromethanesulfonate). LMT.2MsOH/LMTM |

Specifically, LMTM retains TAI activity in vitro and in vivo (Wischik et al, 1996; Harrington et al., 2015; Melis et al., 2015), has superior pharmaceutic properties in terms of solubility and pKa, and is not subject to the absorption limitations of the MT⁺ form (Baddeley et al., 2015)

WO2007/110627 and WO2012/107706 describe dosage units comprising 20 to 300 mg of the DAPTZ compounds described therein e.g. 30 to 200 mg, for example 30 mg, 60 mg, 100 mg, 150 mg, 200 mg. A suitable dose of the DAPTZ compound is suggested in the range of about 100 ng to about 25 mg (more typically about 1 μg to about 10 mg) per kilogram body weight of the subject per day e.g. 100 mg, 3 times daily, 150 mg, 2 times daily, 200 mg, 2 times daily.

WO2008/155533 describes the use of MT compounds for the treatment of mild cognitive impairment (MCI). Total daily doses of from 10 mg to 400 mg are disclosed, preferably administered as twice daily (b.i.d.) or three times daily (t.i.d.) dosage.

WO2018/019823 describes novel regimens for treatment of neurodegenerative disorders utilising methylthioninium (MT)-containing compounds. Briefly, these regimens identified two key factors. The first was in relation to the dosage of MT compounds, and the second was their interaction with symptomatic treatments based on modulation of acetylcholinesterase levels.

In the analysis described in WO2018/019823, low doses of MT compounds (for example 4 mg b.i.d) showed therapeutic benefits when monotherapy was compared against add-on. The efficacy profiles were similar in mild and moderate subjects for most of the measured outcomes.

Furthermore, treatment benefit in AD (according to the trial criteria) was restricted to patients taking LMTM as monotherapy. By contrast, the decline seen at corresponding doses in patients taking LMTM in combination with AD-labelled treatments (acetylcholinesterase inhibitors [AChEls] and\or memantine), who were the majority, was indistinguishable on all parameters from that seen in the control arm.

The potential for LMT compounds to be active at the low dose, and the apparent lack of a dose-response, are discussed in WO2018/019823 and it is hypothesised that there may be a critical threshold for activity at the tau aggregation inhibitor target, and that the effect of higher doses may plateau or may even become negative at brain concentrations above 1 μM (Melis et al., 2015). It had previously been shown that the absorption and distribution of MT to the brain is complex, and likely to be mediated via red cells rather than plasma (Baddeley et al., 2015) providing a route which protects MT from first-pass metabolism. In the same study, MT uptake into red cells was approximately 20-fold higher in vivo when as administered intravenously as LMTM compared with MTC, most likely due to direct red cell uptake of LMT by passive diffusion without need for prior reduction of MT⁺ as is the case for MTC (Baddeley et al., 2015; May et al., 2004).

Based on analyses, and given that lower doses (4 mg twice a day) had a better overall clinical profile than the high dose (100 mg twice a day), WO2018/019823 teaches methods of treatment of neurodegenerative disorders of protein aggregation which comprise oral administration of MT-containing compounds, wherein said administration provides a total of between 0.5 and 20 mg of MT to the subject per day, optionally as a single dose or split into 2 or more doses. For a given daily dosage, WO2018/019823 teaches that more frequent dosing will lead to greater accumulation of a drug.

Other publications using “low dose” or “low dosage” in relation to MT-containing compounds are described in WO2018/019823. For example:

Telch, Michael J., et al. “Effects of post-session administration of methylene blue on fear extinction and contextual memory in adults with claustrophobia.” American Journal of Psychiatry 171.10 (2014): 1091-1098: this publication refers to the use of “low-dose methylene blue” on retention of fear extinction and contextual memory following fear extinction training. The paper reports that “Methylene blue is a diamino phenothiazine drug that at low doses (0.5-4 mg/kg) has neurometabolic-enhancing properties. The dosages used in the publication were 260 mg/day for adult participants, corresponding to a 4 mg/kg dose.

Gonzalez-Lima F and Auchter A (2015) “Protection against neurodegeneration with low-dose methylene blue and near-infrared light”. Front. Cell. Neurosci. 9:179. doi: 10.3389/fncel.2015.00179: this publication discusses the cellular mechanisms mediating the neuroprotective effects of low doses of methylene blue and near-infrared light. It refers to earlier work citing 0.5-4 mg/kg of methylene blue as safe and effective.

Alda, Martin, et al. “Methylene blue treatment for residual symptoms of bipolar disorder: randomised crossover study.” The British Journal of Psychiatry (2016): doi: 10.1192/bjp.bp. 115.173930: this publication described the use of a 15 mg “low dose” of methylene blue as a placebo in a 6-month trial. The “active dose” was 195 mg. In each case the dose was split three times daily.

Rodriguez, Pavel, et al. “Multimodal Randomized Functional MR Imaging of the Effects of Methylene Blue in the Human Brain.” Radiology (2016): 152893: this publication also refers to the ‘known’ pharmacokinetic and side effects of “low-dose” (0.5-4.0 mg/kg) methylene blue, which are contrasted with the effects of dosages greater than 10 mg/kg. The dosages used in the publication were 280 mg/day for adult participants, approximating to a 4 mg/kg dose.

Naylor et al. (1986) “A two-year double-blind crossover trial of the prophylactic effect of methylene blue in manic-depressive psychosis”. Biol. Psychiatry 21:915-920 and Naylor et al. (1987) A controlled trial of methylene blue in severe depressive psychosis. Biol. Psychiatry 22:657-659: these studies used 15 mg/day methylene blue, nominally as a placebo vs. a treatment of 300 mg/day methylene blue. However, in the latter paper the authors proposed that the placebo dosage may act as an antidepressant.

As discussed above, because of their activity in respect of tau aggregation and TDP-43 aggregation, MT-based compounds have also been suggested for the treatment of FTD (see WO2007/110630; WO2007/110627; WO2009/044127; WO2012/107706, all described supra).

WO2018/041739 describes the results of a phase 3 clinical trial investigating the treatment of Frontotemporal dementia (FTD) disease using LMTM.

The results indicated that even a relatively low dose of the MT compound (which was used in the trial as a control) may show benefit in FTD, as compared to the cognitive decline seen in historical controls.

Furthermore, unexpectedly, the results indicated strong interaction effects when MT is co-medicated with AD treatments which modify synaptic neurotransmission in the brain. There appeared significant cognitive benefits in FTD patients taking MT in combination with such AD treatments (e.g. acetylcholinesterase inhibitors and/or memantine) compared to MT alone. WO2018/041739 further describes how MT compounds can be combined with acetylcholinesterase inhibitors and/or memantine without apparent incompatibility.

More recently, WO2020/020751 described a novel pharmacokinetic (PK) model for dosing LMT compounds in patient populations. As expected, there was substantial variability in the MT Cₘₐₓ values across the population for the given low dosage. Analysis of the distribution confirmed the findings in WO2018/019823 that low dosages (4 mg MT b.i.d.) were efficacious (as measured, for example, by reduced decline on the Alzheimer's Disease Assessment Scale-cognitive subscale (ADAS-cog). It further confirmed that monotherapy gave a substantial benefit by this criterion compared to add-on therapy with AChEls and/or memantine (with the mean benefit of between monotherapy and add-on being ˜4 ADAS-cog units over 65 weeks).

However, unexpectedly in view of the previously described lack of any recognisable dose response, the analysis in WO2020/020751 revealed that there exists a concentration response within the low dose treated population. These insights suggested that it was advantageous to adopt a dosing regimen which both maximises the proportion of subjects in which the MT concentration will exceed the Cₘₐₓ or Cₐᵥₑ threshold, and also maximises the expected therapeutic efficacy of LMTM whether it is taken alone or in combination with (or at least preceded by) symptomatic treatments, while nevertheless maintaining a relatively low dose so as to maintain a desirable clinical profile in relation to being well tolerated with minimal side-effects. WO2020/020751 suggests that the minimum dose which achieves all these objectives is at least 20 mg/day, and doses in the range 20-40 mg/day, or 20-60 mg/day would be expected to maximise the therapeutic benefit, although good efficacy, particularly in AD patients not pre-treated with symptomatic treatments, can still be seen at dosages of 100 mg or more.

### DISCLOSURE OF THE INVENTION

The present inventors conducted a randomized, double-blind, placebo-controlled, three-arm, 12-month, safety and efficacy study of hydromethylthionine mesylate (LMTM) monotherapy in subjects with Alzheimer's disease.

Daily doses of LMTM at 8 mg and 16 mg (i.e. 8 mg and 16 mg MT, delivered as LMTM) were compared with a control dose, which comprised dosages of 4 mg MT as MTC, twice weekly. The control dosage was intended to maintain blinding with respect to discolouration of excreta.

The trial results show that administration of 16 mg/day LMTM as monotherapy is effective, with patients in the treatment arm of the study experiencing zero or minimal decline in cognitive and functional measures. As such, the results confirm previous clinical trial results (discussed above) and show that patients receiving 16 mg/day LMTM decline at a rate substantially less than is typical in Alzheimer's. This was seen across a broad range of severity from mild cognitive impairment (MCI) to moderate disease on cognitive and functional endpoints as well as measures of brain atrophy.

Unexpectedly, however, the 4 mg twice weekly ‘control’ dose of MTC also showed therapeutic benefits. Although steady state plasma MT levels in the control group were on average low (significantly lower than in the 16 mg LMTM/day group) in the majority of patients there appears to be equivalent therapeutic benefit even at very low concentration, in patients receiving a twice-weekly dose of MTC 4 mg.

The present inventors have previously shown that LMTM given twice daily produces exposure-dependent benefit on ADAS, ADL and WBV, as monotherapy and as add-on for ADAS and WBV (Schelter et al., 2019; WO2018/019283, vide supra). Therefore, the drug has pharmacological activity with respect to dementia-relevant outcomes. However, it is extremely surprising that such a very low dosing level and low frequency of dosing can produce equivalent benefit.

It is noted that for a given daily dosage, WO2018/019823 teaches that more frequent dosing will lead to greater accumulation of a drug, implying that more frequent dosing is preferred for therapeutic efficacy. The present results are therefore very unexpected.

Without wishing to be bound by theory, this surprising result may be attributable, at least partially, to a significant portion of patients being highly sensitive to quite low levels of the drug, and/or to the relatively selective accumulation of drug administered at low levels in these patients. PK analysis has revealed that the control dose achieves therapeutic blood levels because of an increase in blood levels of the active moiety over time. Blood levels of the drug reveal an exposure response, such that a clear response threshold can be identified.

Without wishing to be bound by theory, the result may also be explained, at least partially, by the timing of the doses being always in the evening, when the brain is undergoing endogenous repair (Alhola et al, 2007; Eugene et al 2015). The present inventors have previously shown in vitro that HMT prevents aggregation at a 1:0.1 stoichiometric ratio of tau: HMT (Al-Hilaly et al., 2018). Therefore, in some patients, even very low levels of MT overnight may be enough to help the brain get rid of aggregated tau.

Irrespective of the mechanism, the disclosure herein indicates that, surprisingly, low doses and/or intermittent administration of low doses of an MT compound can produce substantial clinical benefits. Being able to achieve clinical benefit with a relatively low frequency, low dosage, treatment represents a contribution to the art, since it is desirable to give patients the minimum amount of drug needed to treat the disease.

Thus in one aspect, there is disclosed a method of treatment of a neurodegenerative disease in a subject, which method comprises orally administering to said patient a methylthioninium (MT) containing compound, wherein said administration is at a dosage frequency of less than once daily.

Preferably, the dosage amount is selected such that said low frequency administration provides an amount of MT to the subject that corresponds to an average of between 0.05 and 30 mg of MT per day. In some embodiments, the dosage amount is selected such that said low frequency administration provides an amount of MT to the subject that corresponds to an average of between 0.1 and 20 mg/of MT per day.

In a related aspect, there is disclosed a method of treatment of a neurodegenerative disease in a subject, which method comprises orally administering to said patient a methylthioninium (MT) containing compound, wherein said administration provides an amount of MT to the subject that corresponds to an average of less than 0.5 mg of MT per day.

In some embodiments the neurodegenerative disorder may be AD.

In other embodiments the neurodegenerative disorder may be a neurodegenerative disorder other than AD.

In some embodiments the neurodegenerative disorder may be mild cognitive impairment (MCI). Also provided herein are methods of prophylactic treatment of neurodegenerative disorders of protein aggregation.

Also provided are methylthioninium (MT) containing compounds and compositions thereof, for use in the methods described herein.

These aspects and embodiments will now be described in more detail:

### Dosage Frequency/Timing/Amount

Without wishing to be bound by theory, it has been observed that there is a threshold blood plasma level of active drug moiety (i.e. MT) which is needed to produce a clinical effect. The present inventors have surprisingly discovered that this threshold is significantly lower than previously thought.

Without wishing to be bound by theory, in the methods disclosed herein, prevention of cognitive decline (e.g. in mild to moderate AD patients) appears to occur from a threshold plasma MT level of 0.10 ng/ml. In some embodiments, therefore, administration of an MT compound as described herein provides an amount of MT to the subject which results in a steady state plasma MT level of at least 0.10 ng/ml.

Furthermore, it is thought that an improvement in cognitive function (e.g. in MCI patients) may be associated with a threshold plasma MT level of 0.23 ng/ml. In some embodiments, therefore, administration of the MT compound provides an amount of MT to the subject which results in a steady state plasma MT level of at least 0.23 ng/ml.

In the methods of the invention, the dosage frequency, timing and amount are accordingly chosen so as to result in blood plasma levels of active drug moiety (i.e. MT) which are above the threshold needed to produce the relevant clinical effect.

In some embodiments, the threshold plasma MT levels described above are measured after 12 months of treatment (i.e. at a time point which is 12 months from the first dose of the MT compound). In some embodiments, the threshold plasma MT level described above may be achieved at an earlier time point, i.e. before 12 months of treatment, for example between 4 weeks and 12 months of continuous treatment.

The methods of the invention are partly based on the surprising discovery that infrequent or intermittent dosing (i.e. less than once daily) of a methylthioninium (MT) containing compound can produce therapeutic benefits in patients with neurodegenerative disorders.

In the methods of the invention, treatment with a methylthioninium (MT) containing compound to a patient is carried out under a dosage regimen wherein the compound may be administered to the patient less than once daily.

In other embodiments, the methods of the invention are based on the surprising discovery that even lower amounts of MT than previously thought may provide clinical benefits. Accordingly, in some embodiments, regardless of dosage frequency, administration provides an amount of MT to the subject that corresponds to an average of less than 0.5 mg MT per day.

As used herein, the ‘dosage frequency’ may be defined as an average frequency over a treatment period or duration (e.g. of at least eight weeks). That is, a frequency of less than once daily implies that, over an eight week treatment period, the total number of doses given will be less than the number of days in that period (56). Preferably, the total number of doses given will be substantially less than the number of days in the treatment period.

The treatment period or duration may, for example, be equal to or at least 8 weeks, 3 months, 6 months, or 12 months. Treatment duration is discussed in more detail hereinafter.

In preferred embodiments, the dosage frequency is such that the total number of doses given over a defined treatment period is less than or equal to half the number of days in the treatment period. For example, the total number of doses given over an eight-week treatment period (56 days) is less than or equal to 28. This dosage frequency may be referred to herein as a ≤½ dosage frequency.

In preferred embodiments, the dosage frequency is such that the total number of doses given over a defined treatment period is less than or equal to a third of the number of days in the treatment period. For example, the total number of doses given over an eight-week treatment period (56 days) is less than or equal to 18. This dosage frequency may be referred to herein as a ≤⅓ dosage frequency.

In preferred embodiments, the dosage frequency is such that the total number of doses given over a defined treatment period is less than or equal to a quarter of the number of days in the treatment period. For example, the total number of doses given over an eight-week treatment period (56 days) is less than or equal to 14. This dosage frequency may be referred to herein as a ≤¼ dosage frequency.

In some embodiments, doses of the MT-compound are not administered on consecutive days. In other words, there is a gap of at least one day between each dose of the MT compound.

In alternative embodiments, some doses of the MT-compound may be administered on consecutive days provided that the overall dosage frequency, as defined herein, is not exceeded.

In some embodiments, doses of the MT-compound are administered on no more than two days consecutively. In some embodiments, doses of the MT-compound are administered on no more than three days consecutively.

In some embodiments, dosage may be at regular intervals. For example, a dose may be administered every other day (once every two days), once every three days, or once or twice weekly, on fixed day(s) of each week.

In other embodiments, dosage may be irregular or intermittent. For example a dose may be administered once, twice or three times weekly, on a varying or random schedule i.e. on varying or random days each week.

The dosage frequency may be, for example, every two days, every three days, every four days, every five days, every six days, weekly, twice weekly, or thrice (3×) weekly.

In some embodiments, the dosage frequency is selected from: every other day; once weekly; or twice weekly.

In some embodiments, the MT compound is administered to the patient every other day.

In some embodiments, the MT compound is administered to the patient twice weekly, on a varying schedule.

In some embodiments, the MT compound is administered to the patient twice weekly, on a fixed schedule (i.e. on fixed days each week).

In some embodiments, the MT compound is administered to the patient once weekly, on a varying schedule.

In some embodiments, the MT compound is administered to the patient once weekly, on a fixed schedule

Without wishing to be bound by theory, it is thought that evening dosing may be more effective, at least in some patients.

In some embodiments, the MT compound is administered in the evening.

In some embodiments, at least some doses of the MT compound are administered in the evening.

As defined herein, the average amount of MT provided by a given dosage amount, at a given dosage frequency, is calculated by dividing the amount of MT provided in each dose of the MT compound, by the number of days in each dosage window. For example, as shown in Table 1:

| Dosage frequency | Dosage amount | Window | average MT/day | |
|---|---|---|---|---|
| Daily | X mg MT every | 1 day | X | mg |
| Every other day | X mg MT every | 2 days | X/2 | mg |
| Every 3 days | X mg MT every | 3 days | X/3 | mg |
| Every 4 days | X mg MT every | 4 days | X/4 | mg |
| Thrice weekly | 3 × X mg MT every | 7 days | 3X/7 | mg |
| Twice weekly | 2 × X mg MT every | 7 days | 2X/7 | mg |
| Once weekly | X mg MT every | 7 days | X/7 | mg |

In some embodiments, administration provides an amount of MT to the subject that corresponds to an average amount per day of from around any of 0.05, 0.1, 0.2, 0.3, 0.4, 0.5, 1, 1.5, and 2 mg to around any of 2.5, 3, 5, 6, 7, 8, 9, 10, 11, 12, 13, 14, 15, 16, 17, 18, 19, 20, 25, and 30 mg.

In some embodiments, the average amount of MT per day is from 0.05 to 30 mg.

In some embodiments, the average amount of MT per day is from 0.05 to 25 mg.

In some embodiments, the average amount of MT per day is from 0.05 to 20 mg.

In some embodiments, the average amount of MT per day is from 0.1 to 30 mg.

In some embodiments, the average amount of MT per day is from 0.1 to 25 mg.

In some embodiments, the average amount of MT per day is from 0.1 to 20 mg.

In some embodiments, the average amount of MT per day is from 0.2 to 30 mg.

In some embodiments, the average amount of MT per day is from 0.2 to 25 mg.

In some embodiments, the average amount of MT per day is from 0.2 to 20 mg.

In some embodiments, the average amount of MT per day is from 0.2 to 10 mg.

In some embodiments, the average amount of MT per day is from 0.2 to 5 mg.

In some embodiments, the average amount of MT per day is from 0.2 to 3 mg.

In some embodiments, the average amount of MT per day is from 0.3 to 10 mg.

In some embodiments, the average amount of MT per day is from 0.3 to 5 mg.

In some embodiments, the average amount of MT per day is from 0.3 to 3 mg.

In some embodiments, the average amount of MT per day is from 0.4 to 10 mg.

In some embodiments, the average amount of MT per day is from 0.4 to 5 mg.

In some embodiments, the average amount of MT per day is from 0.4 to 3 mg.

In some embodiments, the average amount of MT per day is from 0.5 to 10 mg.

In some embodiments, the average amount of MT per day is from 0.5 to 5 mg.

In some embodiments, the average amount of MT per day is from 0.5 to 3 mg.

In some embodiments, the average amount of MT per day is less than 0.5 mg.

In some embodiments, the average amount of MT per day is from 0.05 to less than 0.5 mg.

In some embodiments, the average amount of MT per day is from 0.1 to less than 0.5 mg.

In some embodiments, the average amount of MT per day is from 0.05 to 0.49 mg.

In some embodiments, the average amount of MT per day is from 0.10 to 0.49 mg.

In one embodiment the average amount of MT per day is from 0.75 to 1.25 mg/day, or about 1 mg/day, for example delivered as MTC.

As will be appreciated, the appropriate dosage amount will depend on the dosage frequency. The dosage amount is preferably chosen such that the amount of MT provided to the subject corresponds to the desired average amount of MT per day, as defined above.

In some embodiments, the amount of MT provided by each dose of the methylthioninium (MT) containing compound is from around any of 0.05, 0.1, 0.2, 0.4, 0.5, 0.6, 0.8, 1.0, 1.2, and 1.4 mg MT to around any of 5, 10, 20, 40, 50, 60, 70, 80, 100, 120 and 140 mg MT.

In some embodiments, the amount of MT per dose is from 0.1 to 100 mg.

In some embodiments, the amount of MT per dose is from 0.1 to 50 mg.

In some embodiments, the amount of MT per dose is from 0.2 to 100 mg.

In some embodiments, the amount of MT per dose is from 0.2 to 50 mg.

In some embodiments, the amount of MT per dose is from 0.2 to 20 mg.

In some embodiments, the amount of MT per dose is from 0.5 to 50 mg.

In some embodiments, the amount of MT per dose is from 0.5 to 20 mg.

In some embodiments, the amount of MT per dose is from 0.5 to 10 mg.

In some embodiments, the amount of MT per dose is from 0.5 to 5 mg.

In some embodiments, the dosage frequency is ≤½ and the amount of MT provided in each dose is between 0.4 and 40 mg.

In some embodiments, the dosage frequency is every other day and the amount of MT provided in each dose is between 0.4 and 40 mg.

In some embodiments, the dosage frequency is ≤½ and the amount of MT provided in each dose is between 0.1 and 60 mg.

In some embodiments, the dosage frequency is every other day and the amount of MT provided in each dose is between 0.1 and 60 mg.

In some embodiments, the dosage frequency is ≤⅓ and the amount of MT provided in each dose is between 0.6 and 60 mg.

In some embodiments, the dosage frequency is every 3 days and the amount of MT provided in each dose is between 0.6 and 60 mg.

In some embodiments, the dosage frequency is ≤⅓ and the amount of MT provided in each dose is between 0.15 and 90 mg.

In some embodiments, the dosage frequency is every 3 days and the amount of MT provided in each dose is between 0.15 and 90 mg.

In some embodiments, the dosage frequency is ≤¼ and the amount of MT provided in each dose is between 0.8 and 80 mg.

In some embodiments, the dosage frequency is every 4 days and the amount of MT provided in each dose is between 0.8 and 80 mg.

In some embodiments, the dosage frequency is ≤¼ and the amount of MT provided in each dose is between 0.2 and 120 mg.

In some embodiments, the dosage frequency is every 4 days and the amount of MT provided in each dose is between 0.2 and 120 mg.

In some embodiments, the dosage frequency is every 5 days and the amount of MT provided in each dose is between 1 and 100 mg.

In some embodiments, the dosage frequency is every 6 days and the amount of MT provided in each dose is between 1.2 and 120 mg.

In some embodiments, the dosage frequency is weekly and the amount of MT provided in each dose is between 1.4 and 140 mg.

In some embodiments, the dosage frequency is every 5 days and the amount of MT provided in each dose is between 0.25 and 150 mg.

In some embodiments, the dosage frequency is every 6 days and the amount of MT provided in each dose is between 0.3 and 180 mg.

In some embodiments, the dosage frequency is weekly and the amount of MT provided in each dose is between 0.35 and 210 mg.

In some embodiments, the dosage frequency is twice weekly and the amount of MT provided in each dose is between 0.7 and 70 mg.

In some embodiments, the dosage frequency is thrice (three times) weekly and the amount of MT provided in each dose is between 0.5 and 50 mg.

In some embodiments, the dosage frequency is twice weekly and the amount of MT provided in each dose is between 0.2 and 100 mg.

In some embodiments, the dosage frequency is thrice (three times) weekly and the amount of MT provided in each dose is between 0.15 and 70 mg.

### Methylthioninium Moiety

| Structure | 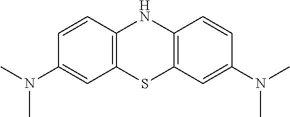 | 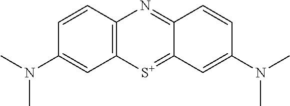 |
|---|---|---|
| IUPAC | N3,N3,N7,N7-tetramethyl-10H- | N3,N3,N7,N7- |
| | phenothiazine-3,7-diamine | tetramethylphenothiazin-5-ium- |
| | | 3,7-diamine |
| Composition | Formula Weight: 285.41(1) | Formula Weight: 284.40(1) |
| | Exact Mass: 285.1299683(1) | Exact Mass: 284.1215947(1) |
| | Formula: C₁₆H₁₉N₃S | Formula: C₁₆H₁₈N₃S |
| | Composition: C 67.33% H 6.71% | Composition: C 67.57% H |
| | N 14.72% S 11.23% | 6.38% N 14.78% S 11.27% |
| Synonyms | leucomethylthioninium (LMT) | oxidized methylthioninium (MT⁺) |
| | hydromethylthionine (HMT) | |

The MT-containing compounds used in the present invention can contain MT in either reduced or oxidised form. The “MT” is the active ingredient, which is to say that it is present to provide the recited therapeutic effect. Specifically, the compounds may comprise either of the MT moieties described above. The MT moieties per se described above are not stable. They will therefore be administered as MT compounds—for example LMT or MT⁺ salts.

MT⁺ salts will generally include one or more anionic counter ions (X⁻) to achieve electrical neutrality. The compounds may be hydrates, solvates, or mixed salts of the MT⁺ salt.

LMT containing compounds will generally be stabilised, for example by the presence of one or more protic acids e.g. two protic acids.

The MT content of such salts can be readily calculated by those skilled in the art based on the molecular weight of the compound, and the molecular weight of the MT moiety. Examples of such calculations are given herein.

### LMT Compounds

In some embodiments, the MT compound is preferably an LMT compound.

In some embodiments, the MT compound is an “LMTX” compound of the type described in WO2007/110627 or WO2012/107706.

Thus, the compound may be selected from compounds of the following formula, or hydrates or solvates thereof:

| | |
|---|---|
| 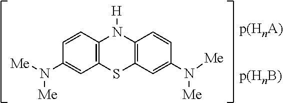 | Options: p = 1, 2 q = 0, 1 n = 1, 2 (p + q) × n = 2 |

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

| | |
|---|---|
| 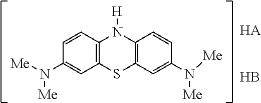 | when: p = 1 q = 1 n = 1 (1 + 1) × 1 = 2 |

However preferably the salt is not a mixed salt, and has the following formula:

| | |
|---|---|
| 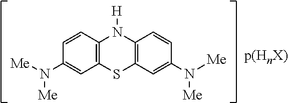 | when: p = 1, 2 n = 1, 2 p × n = 2 |

wherein each of HₙX is a protic acid, such as a di-protic acid or mono-protic acid.

In one embodiment the salt has the following formula, where H₂A is a di-protic acid:

| | |
|---|---|
| 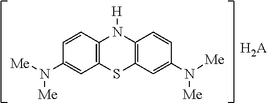 | when: p= 1 q = 0 n = 2 (1 + 0) × 2 = 2 |

Preferably the salt has the following formula which is a bis monoprotic acid:

| | |
|---|---|
| 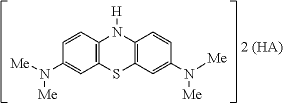 | when: p = 2 q= 0 n = 1 (2 + 0) × 1 = 2 |

Examples of protic acids which may be present in the LMTX compounds used herein include: Inorganic acids: hydrohalide acids (e.g., HCl, HBr), nitric acid (HNO₃), sulphuric acid (H₂SO₄)

Organic acids: carbonic acid (H₂CO₃), acetic acid (CH₃COOH), methanesulfonic acid, 1,2-Ethanedisulfonic acid, ethansulfonic acid, naphthalenedisulfonic acid, p-toluenesulfonic acid,

Preferred acids are monoprotic acid, and the salt is a bis(monoprotic acid) salt.

A preferred MT compound is LMTM:

| | | | |
|---|---|---|---|
| 1 | 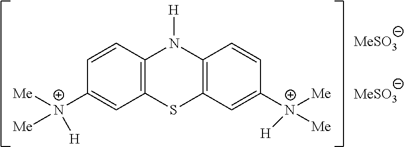 | LMT.2MsOH (LMTM) | 477.6 (1.67) |

The anhydrous salt has a molecular weight of around 477.6. Based on a molecular weight of 285.1 for the LMT core, the weight factor for using this MT compound in the invention is 1.67. By “weight factor” is meant the relative weight of the pure MT containing compound vs. the weight of MT which it contains.

Other weight factors can be calculated for example MT compounds herein, and the corresponding dosage ranges can be calculated therefrom.

Therefore, the present invention embraces dosage regimes providing an average amount of around 0.08 to 50 mg/day of LMTM, including dosage regimes providing an average amount of around 0.17 to 33 mg/day of LMTM.

More preferably the average amount of LMTM provided to the subject is from any of 0.1, 0.2, 0.3, 0.4, 0.5 to any of 2, 3, 5, 10, 20, 25, or 50 mg per day.

In some embodiments, the method comprises administration of about 1 mg LMTM, every other day. In some embodiments, the method comprises administration of about 3.5 mg LMTM, every other day. In some embodiments, the method comprises administration of about 7 mg LMTM, every other day. In some embodiments, the method comprises administration of about 14 mg LMTM, every other day. In some embodiments, the method comprises administration of about 27 mg LMTM, every other day.

In some embodiments, the method comprises administration of about 2 mg LMTM, twice weekly.

In some embodiments, the method comprises administration of about 7 mg LMTM, twice weekly.

In some embodiments, the method comprises administration of about 14 mg LMTM, twice weekly. In some embodiments, the method comprises administration of about 27 mg LMTM, twice weekly.

In some embodiments, the method comprises administration of about 2 mg LMTM, once weekly.

In some embodiments, the method comprises administration of about 7 mg LMTM, once weekly.

In some embodiments, the method comprises administration of about 14 mg LMTM, once weekly. In some embodiments, the method comprises administration of about 27 mg LMTM, once weekly.

Other example LMTX compounds are as follows. Their molecular weight (anhydrous) and weight factor is also shown:

| | | | MW |
|---|---|---|---|
| | | | (weight |
| | Compound | Abbreviation | factor) |
| 2 | 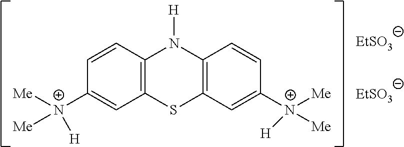 | LMT.2EsOH | 505.7 (1.77) |
| 3 | 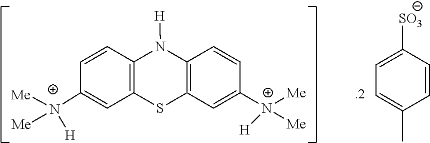 | LMT.2TsOH | 629.9 (2.20) |
| 4 | 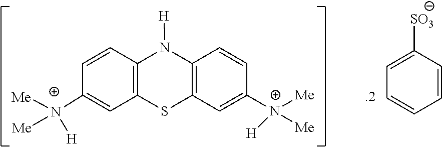 | LMT.2BSA | 601.8 (2.11) |
| 5 | 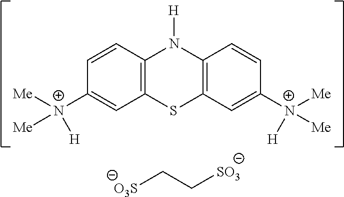 | LMT.EDSA | 475.6 (1.66) |
| 6 | 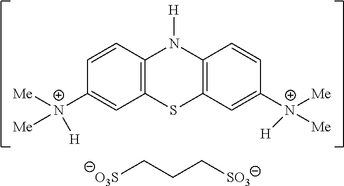 | LMT.PDSA | 489.6 (1.72) |
| 7 | 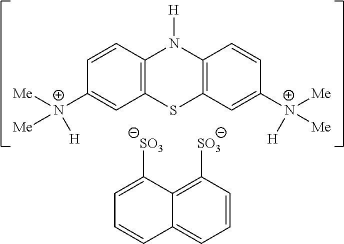 | LMT.NDSA | 573.7 (2.01) |
| 8 | 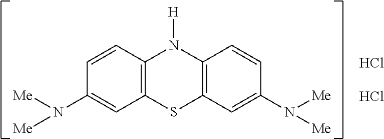 | LMT.2HCI | 358.33 (1.25) |

The dosages described herein with respect to MT thus apply mutatis mutandis for these MT containing compounds, as adjusted for their molecular weight.

### Oxidised MT Compounds

In another embodiment the MT compound is an MT⁺ compound.

Preferably the MT compound is an MT⁺ compound of the type described in WO96/30766 or WO2007/110630.

Thus, the compound may be selected from compounds of the following formula, or hydrates, solvates, or mixed salts thereof:

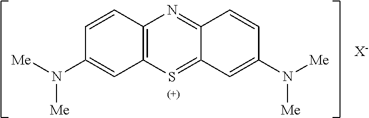

Where X⁻ is an anionic counter ion.

In some embodiments of the present invention the MT⁺ compound is MTC, for example a “high purity” MTC as described below.

In some embodiments of the present invention the MT⁺ compound is not MTC.

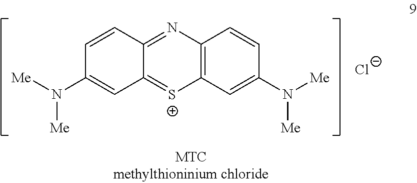

As explained in WO2011/036561 and WO2011/036558, MTC occurs in a number of polymorphic forms having different levels of hydration.

In some embodiments of the present invention, the MT⁺ compound is a high purity MTC. In this context ‘high purity’ is defined by one or more of the criteria set out below.

In some embodiments, the MTC has a purity of greater than 97%.

In some embodiments, the MTC has a purity of greater than 98%.

In some embodiments, the MTC has a purity of greater than 99%.

In some embodiments, the MTC has less than 2% Azure B as impurity.

In some embodiments, the MTC has less than 1% Azure B as impurity.

In some embodiments, the MTC has less than 0.5% Azure B as impurity.

In some embodiments, the MTC has less than 0.1% Azure B as impurity.

In some embodiments, the MTC has less than 0.15% Azure A as impurity.

In some embodiments, the MTC has less than 0.10% Azure A as impurity.

In some embodiments, the MTC has less than 0.05% Azure A as impurity.

In some embodiments, the MTC has less than 0.15% Azure C as impurity.

In some embodiments, the MTC has less than 0.10% Azure C as impurity.

In some embodiments, the MTC has less than 0.05% Azure C as impurity.

In some embodiments, the MTC has less than 0.13% MVB (Methylene Violet Bernstein) as impurity.

In some embodiments, the MTC has less than 0.05% MVB as impurity.

In some embodiments, the MTC has less than 0.02% MVB as impurity.

All percentage purities recited herein are by weight unless otherwise specified.

In some embodiments, the MTC has an elementals purity that is better than that specified by the European Pharmacopeia (EP).

As used herein, the term ‘elementals purity’ pertains to the amounts of the twelve (12) metals specified by the European Pharmacopeia: Al, Cd, Cr, Cu, Sn, Fe, Mn, Hg, Mo, Ni, Pb, and Zn. The current edition of the European Pharmacopeia (8th Edition, supplementum 8.8) specifies the following limits for these metals:

European Pharmacopeia 8.8 (EP8.8)

| Element | Maximum content (μg/g) |
|---|---|
| Aluminium (Al) | 100 |
| Cadmium (Cd) | 1 |
| Chromium (Cr) | 100 |
| Copper (Cu) | 300 |
| Tin (Sn) | 10 |
| Iron (Fe) | 200 |
| Manganese (Mn) | 10 |
| Mercury (Hg) | 1 |
| Molybdenum (Mo) | 10 |
| Nickel (Ni) | 10 |
| Lead (Pb) | 10 |
| Zinc (Zn) | 100 |

In one embodiment, the MTC has an elementals purity (e.g. for each of Al, Cd, Cr, Cu, Sn, Fe, Mn, Hg, Mo, Ni, Pb, and Zn) which is equal to or better than (i.e. lower than) the EP8.8 values set out in the table above.

In one embodiment, the MTC has an elementals purity which is equal to or better than 0.9 times the EP8.8 values set out in the table above.

In one embodiment, the MTC has an elementals purity which is equal to or better than 0.8 times the EP8.8 values set out in the table above.

In one embodiment, the MTC has an elementals purity which is equal to or better than 0.7 times the EP8.8 values set out in the table above.

In one embodiment, the MTC has an elementals purity which is equal to or better than 0.5 times the EP8.8 values set out in the table above.

(For example, 0.5 times the EP8.8 values as set out above are 50 μg/g Al, 0.5 μg/g Cd, 50 μg/g Cr, etc.)

In one embodiment the MTC has a chromium level that is equal to or better than (i.e. lower than) 100 μg/g.

In one embodiment the MTC has a chromium level that is equal to or better than (i.e. lower than) 10 μg/g.

In one embodiment the MTC has a copper level that is equal to or better than (i.e. lower than) 300 μg/g.

In one embodiment the MTC has a copper level that is equal to or better than (i.e. lower than) 100 μg/g.

In one embodiment the MTC has a copper level that is equal to or better than (i.e. lower than) 10 μg/g.

In one embodiment the MTC has an iron level that is equal to or better than (i.e. lower than) 200 μg/g.

In one embodiment the MTC has an iron level that is equal to or better than (i.e. lower than) 100 μg/g.

All plausible and compatible combinations of the above purity grades are disclosed herein as if each individual combination was specifically and explicitly recited.

In particular embodiments, the MTC is a high purity MTC wherein ‘high purity’ is characterised by a purity of greater than 98% and one or more of the following:
(i) less than 2% Azure B as impurity;
(ii) less than 0.13% MVB (Methylene Violet Bernstein) as impurity; or
(iii) an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by a purity of greater than 98% and one or more of the following:
(i) less than 1% Azure B as impurity;
(ii) less than 0.15% Azure A as impurity;
(iii) less than 0.15% Azure C as impurity;
(iv) less than 0.13% Methylene Violet Bernthsen (MVB) as impurity;
(v) an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by a purity of greater than 98% and one or more of the following:
(i) less than 1% Azure B as impurity;
(ii) less than 0.15% Azure A as impurity;
(iii) less than 0.15% Azure C as impurity;
(iv) less than 0.05% Methylene Violet Bernthsen (MVB) as impurity; or
(v) an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by at least 98% purity and less than 1% Azure B as impurity.

In particular embodiments, the MTC is a high purity MTC wherein high-purity is characterised by:
(i) at least 98% purity
(i) less than 1% Azure B as impurity; and
(ii) an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by at least 98% purity and an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

Methods for the production of ‘high purity’ diaminophenothiazinium compounds, including MTC, are described, for example, in WO2006/032879 and WO2008/007074 (WisTa Laboratories Ltd) and in WO2008/006979 (Provence Technologies).

A preferred MTC polymorph for use in the methods and compositions described herein is ‘form A’ described in WO2011/036561 which is a pentahydrate, at a “high purity” described above. That has a molecular weight of around 409.9. Based on a molecular weight of 284.1 for the MT⁺ core, the weight factor for using this MT compound in the invention is 1.44.

Other weight factors can be calculated for example MT compounds herein, and the corresponding dosage ranges can be calculated therefrom.

Therefore the invention the present invention embraces dosage regimes providing an average amount of around 0.07 to 43 mg/day of MTC.5H₂O, including dosage regimes providing an average amount of around 0.14 to 29 mg/day of MTC.5H₂O.

In some embodiments the average amount of MTC.5H₂O provided to the subject is from any of 0.1, 0.2, 0.3, 0.4, 0.5, 0.6, 0.7, 0.8 to any of 2, 3, 5, 6, 10, 12, 20, 24, 35, or 43 mg per day.

In some embodiments, the method comprises administration of about 2 mg MTC (e.g. MTC.5H₂O), every other day. In some embodiments, the method comprises administration of about 6 mg MTC (e.g. MTC.5H₂O), every other day. In some embodiments, the method comprises administration of about 12 mg MTC, every other day. In some embodiments, the method comprises administration of about 24 mg MTC, every other day.

In some embodiments, the method comprises administration of about 2 mg MTC (e.g. MTC.5H₂O), twice weekly. In some embodiments, the method comprises administration of about 6 mg MTC (e.g. MTC.5H₂O), twice weekly. In some embodiments, the method comprises administration of about 12 mg MTC, twice weekly. In some embodiments, the method comprises administration of about 24 mg MTC, twice weekly.

In some embodiments, the method comprises administration of about 2 mg MTC (e.g. MTC.5H₂O), once weekly. In some embodiments, the method comprises administration of about 6 mg MTC (e.g. MTC.5H₂O), once weekly. In some embodiments, the method comprises administration of about 12 mg MTC, once weekly. In some embodiments, the method comprises administration of about 24 mg MTC, once weekly.

Other example MT compounds are described in WO2007/110630. Their molecular weight (anhydrous) and weight factor is also shown:

| | | | MT⁺ anhydrous |
|---|---|---|---|
| | Compound | Molecular weight | weight factor |
| 10 | MTC•0.5ZnCl₂ | 388.0 | 1.36 |
| 11 | MTI | 411.3 | 1.45 |
| 12 | MTI•HI | 539.2 | 2.73 |
| 13 | MT•NO₃ | 346.4 | 1.22 |

The dosages described herein with respect to MT thus apply mutatis mutandis for these MT containing compounds, as adjusted for their molecular weight, and for choice of hydrate if used. For example MTC.0.5ZnCl₂ (also referred to as ‘METHYLENE BLUE ZINC CHLORIDE DOUBLE SALT; CI 52015) may be obtained commercially as a monohydrate by several suppliers, which would have a molecular weight higher by 18, and correspondingly altered weight factor. MTI is reportedly available as a hemihydrate.

### Adsorption Factors

The present inventors have previously determined that dosing with LMTX salts permits more efficient adsorption, compared with MT⁺ salts. Typically MT adsorption may be around 1.5× greater when delivered as an LMTX salt as opposed to an MT⁺ salts. This 1.5-fold factor may be termed herein an “adsorption factor”.

Therefore in certain embodiments of the invention, the dosed amount of MT⁺ salt may be higher than when using LMTX salt to achieve a similar plasma concentration.

Without wishing to be bound by theory, however, it is thought that at the very low doses and reduced dosage frequencies envisaged by the present invention, adsorption factors may be less significant as, at very low levels, substantially all MT is converted to HMT in vivo.

Any of the MT compounds described herein, may be formulated with a reducing agent. In particular, MT⁺ salts such as MTC may be formulated with a reducing agent such as ascorbate, and then lyophilized (as described in WO02/055720). This may improve adsorption of the MT delivered by the compound.

In the various aspects of the invention described herein (as they relate to an MT-containing compound) the MT-containing compound may optionally be any of those compounds described above:

In one embodiment, it is compound 1.

In one embodiment, it is compound 2.

In one embodiment, it is compound 3.

In one embodiment, it is compound 4.

In one embodiment, it is compound 5.

In one embodiment, it is compound 6.

In one embodiment, it is compound 7.

In one embodiment, it is compound 8.

In one embodiment, it is compound 9.

In one embodiment, it is compound 10.

In one embodiment, it is compound 11.

In one embodiment, it is compound 12.

In one embodiment, it is compound 13.

Or the compound may be a hydrate, solvate, or mixed salt of any of these.

### Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention may be much lower than was hitherto understood in the art.

The invention also embraces treatment as a prophylactic measure is also included.

Thus the invention also provides a method of prophylaxis of a neurodegenerative disorders of protein aggregation in a subject,
which method comprises orally administering to said patient a methylthioninium (MT) containing compound,
wherein said administration is at a dosage frequency of less than once daily;
   and wherein said administration provides an amount of MT to the subject that corresponds to an average of between 0.05 mg and 30 mg MT per day, preferably between 0.1 mg and 20 mg MT per day.

The invention also provides a method of prophylaxis of a neurodegenerative disorders of protein aggregation in a subject,
which method comprises orally administering to said patient a methylthioninium (MT) containing compound,
wherein said administration provides an amount of MT to the subject that corresponds to less than 0.5 mg MT per day.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

### Combination Treatments and Monotherapy

The term “treatment” includes “combination” treatments and therapies, in which two or more treatments or therapies for the same neurodegenerative disorder of protein aggregation, are combined, for example, sequentially or simultaneously. These may be symptomatic or disease modifying treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be an agent which is MT-containing compound at the specified dosage in combination with an agent which is an inhibitor of amyloid precursor protein to beta-amyloid (e.g., an inhibitor of amyloid precursor protein processing that leads to enhanced generation of beta-amyloid).

In other embodiments the treatment is a “monotherapy”, which is to say that the MT-containing compound is not used in combination (within the meaning discussed above) with another active agent for treating the same neurodegenerative disorder of protein aggregation in the subject.

As explained below, in the present invention, when treating AD at least, it is preferred that the treatment does not include administration of either or both of: an acetylcholinesterase inhibitor or an N-methyl-D-aspartate receptor antagonist. The MT-compound based treatment of AD may optionally be a monotherapy.

### Duration of Treatment

For treatment of the neurodegenerative disorder of protein aggregation described herein, a treatment regimen based on the low dose MT compounds will preferably extend over a sustained period of time. The particular duration would be at the discretion of the physician.

For example, the duration of treatment may be:

At least 1, 2, 3, 4, 5, 6, 7, 8, 9, 10, 11, 12 months, or longer.

At least 2, 3, 4, 5 years, or longer.

Between 6 and 12 months.

Between 1 and 5 years.

Where the disorder is AD, duration may be such as to achieve any one or more of:
A 3, 4 or 5-point improvement on the 11-item Alzheimer's Disease Assessment Scale-cognitive subscale (ADAS-cog) over a 52-week period;
4, 5 or 6 point improvement on the 23-item Alzheimer's Disease Cooperative Study Activities of Daily Living (ADCS-ADL) over a 52-week period;
A reduction in the increase of Lateral Ventricular Volume (LVV), as measured by the Ventricular Boundary Shift Integral (VBSI) of 1 or 2 cm³ over a 52-week period.
A decrease in annualized rate of whole brain atrophy on brain MRI using BSI.

For prophylaxis, the treatment may be ongoing.

In all cases the treatment duration will generally be subject to advice and review of the physician.

### Pharmaceutical Dosage Forms

Preferably the MT compound of the invention is administered in the form of a pharmaceutical composition. Preferably such a composition comprises a compound as described herein, and a pharmaceutically acceptable carrier or diluent.

In some embodiments, the composition is a pharmaceutical composition (e.g., formulation, preparation, medicament) comprising a compound as described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

In some embodiments, the composition is in the form of a dosage unit (e.g., a pharmaceutical tablet or capsule) comprising an MT compound as described herein (e.g., obtained by, or obtainable by, a method as described herein; having a purity as described herein; etc.), and a pharmaceutically acceptable carrier, diluent, or excipient.

The “MT compound”, although present in relatively low amount, is the active agent of the dosage unit, which is to say is intended to have the therapeutic or prophylactic effect in respect of a neurodegenerative disorder of protein aggregation. Rather, the other ingredients in the dosage unit will be therapeutically inactive e.g. carriers, diluents, or excipients. Thus, preferably, there will be no other active ingredient in the dosage unit, no other agent intended to have a therapeutic or prophylactic effect in respect of a disorder for which the dosage unit is intended to be used.

In some embodiments, the dosage unit is a tablet.

In some embodiments, the dosage unit is a capsule.

In some embodiments, said capsules are gelatine capsules.

In some embodiments, said capsules are HPMC (hydroxypropylmethylcellulose) capsules.

In some embodiments, the amount of MT in the unit is 0.1 to 10 mg.

In some embodiments, the amount of MT in the unit is 0.05 to 10 mg.

In some embodiments, the amount of MT in the unit is 0.05 to 5 mg.

An example dosage unit may contain 1 to 10 mg of MT.

A further example dosage unit may contain 2 to 9 mg of MT.

A further example dosage unit may contain 3 to 8 mg of MT.

A further preferred dosage unit may contain 3.5 to 7 mg of MT.

A further preferred dosage unit may contain 4 to 6 mg of MT.

In some embodiments, the amount is about 0.05, 0.1, 0.5, 1, 1.5, 2, 2.5, 3, 3.5, 4, 5, 6, 7, 8, 9, 10 mg of MT.

Using the weight factors described or explained herein, one skilled in the art can select appropriate amounts of an MT containing compound to use in oral formulations.

As explained above, the MT weight factor for LMTM is 1.67. Since it is convenient to use unitary or simple fractional amounts of active ingredients, non-limiting example LMTM dosage units may include 0.5, 1, 1.5, 2, 2.5, 3, 3.5, 4, 5, 6, 7, 8, 9, 10, 15, 16, 17, 18 mg etc.

As explained above, the MT weight factor for MTC.5H₂O is 1.44. Since it is convenient to use unitary or simple fractional amounts of active ingredients, non-limiting example MTC.5H₂O dosage units may include 0.5, 1, 1.5, 2, 2.5, 3, 3.5, 4, 5, 6, 7, 8, 9, 10, 15, 18, 20 mg etc.

The surprising results leading to the present invention suggest that lower doses of MT compounds than previously thought may be useful in the treatment of neurodegenerative disorders. Dosage units containing very low amounts of the MT compound may therefore be beneficial. Accordingly, in another aspect of the present invention, there is provided a pharmaceutical composition comprising an MT compound as defined herein, and a pharmaceutically acceptable carrier or diluent, optionally in the form of a dosage unit, wherein the amount of MT in the composition or unit is less than 0.5 mg.

In some embodiments, the amount of MT in the composition or unit is from any one of 0.05 mg, 0.1 mg, 0.2 mg, and 0.3 mg to any one of 0.45, 0.46, 0.47, 0.48, 0.49, and about 0.5 mg.

### Oral Dosage Forms

The MT compound of the present invention, or pharmaceutical composition comprising it, is preferably administered to a subject/patient orally.

### Nutraceutical Compositions

The “nutraceutical compositions” of the invention comprise a low dose of MT compound, as described herein, in combination with one or more nutrients in an edible form (for example an oral dosage form).

The novel nutraceutical compositions of the invention can find use as supplements to food and beverages, and as pharmaceutical compositions.

“Nutrients” as used herein refers to the components of nutraceutical compositions that serve a biochemical and/or physiological role in the human or animal body. “Nutrients” includes such substances as vitamins, minerals, trace elements, micronutrients, antioxidants and the like, as well as other bioactive materials, such as enzymes, or compounds biosynthetically produced by human or animal enzymes; as well as herbs and herbal extracts; fatty acids, amino acids and derivatives thereof.

“Edible form” denotes a composition that can be ingested directly or converted to an ingestible form, such as, by dissolving in water.

Alternatively, the nutraceutical composition can be in the form of a food or drink, such as a defined portion of a foodstuff (which term includes both food or drink) supplemented with the defined dosage of MT compound. These foodstuffs will typically comprise one or more of a fat, a protein, or a carbohydrate.

The term “nutraceutical’ as used herein denotes a usefulness in both the nutritional and pharmaceutical field of application, and the disclosure herein relating to pharmaceutical dosage forms applies mutatis mutandis to the nutraceutical compositions.

Oral dosage forms particularly suitable for nutraceutical compositions are well known in the art and described in more detail elsewhere herein. They include powders, capsules, pills, tablets, caplets, gelcaps, and defined portions of edible food items. Liquid forms include solutions or suspensions. General examples of dosage forms and nutraceutical forms are given, for example in WO2010/078659.

Some examples of nutrients useful in the compositions of the present invention are as follows. Any combination of these nutrients is envisaged by the present invention:

### Vitamins

It is reported that B-vitamin supplementation (folic acid [folate, vitamin B₉], vitamin B₁₂, vitamin B₆) can slow the atrophy of specific brain regions that are a key component of the AD process and that are associated with cognitive decline. This is particularly the case for elderly subjects with high homocysteine levels (Douaud, Gwenaëlle, et al. “Preventing Alzheimer's disease-related gray matter atrophy by B-vitamin treatment.” Proceedings of the National Academy of Sciences 110.23 (2013): 9523-9528; see also Quadri, Pierluigi, et al. “Homocysteine, folate, and vitamin B₁₂ in mild cognitive impairment, Alzheimer disease, and vascular dementia.” The American journal of clinical nutrition 80.1 (2004): 114-122; Rosenberg I H, Miller J W. Nutritional factors in physical and cognitive functions of elderly people. The American journal of clinical nutrition. 1992 Jun. 1; 55 (6): 1237S-1243S.).

It has been suggested that, along with other antioxidants (see below), vitamin C may have utility in protecting neural tissue, as well as potentially decreasing β-amyloid generation and acetylcholinesterase activity and prevents endothelial dysfunction by regulating nitric oxide (see e.g. Heo J H, Hyon-Lee, Lee K M. The possible role of antioxidant vitamin C in Alzheimer's disease treatment and prevention. American Journal of Alzheimer's Disease & Other Dementias. 2013 March; 28 (2): 120-5).

It has also been suggested that Vitamin E supplementation may have a role to play in AD treatment (see e.g. Mangialasche, Francesca, et al. “Serum levels of vitamin E forms and risk of cognitive impairment in a Finnish cohort of older adults.” Experimental Gerontology 48.12 (2013): 1428-1435).

### Micronutrients, Antioxidants

Micronutrients or antioxidants, such as polyphenols, have been reported to have benefits in relation to protection or treatment of age-related diseases including neurodegenerative ones, particularly cognitive impairment and AD.

Micronutrients and/or antioxidants which may be utilised in the nutraceutical compositions described herein include the flavonoids shown in the Table below (reproduced from Mecocci, Patrizia, et al. “Nutraceuticals in cognitive impairment and Alzheimer's disease.” Frontiers in Pharmacology 5:147 (2014)):

Flavonoid chemical subgroups and relative food sources:

| Groups | Molecules | Food source |
|---|---|---|
| FLAVANOLS | Catechin, epicatechin, | Cocoa and chocolate, |
| | epigallocathechin, | green tea, grapes |
| | epigallocatechin | |
| | gallate (EGCG) | |
| FLAVONOLS | Kaempferol, quercetin | Onions, apples, green |
| | | tea, capers, leeks, |
| | | broccoli |
| FLAVONES | Luteolin, apigenin | Celery, parsley, |
| | | rosemary |
| ISOFLAVONES | Daidzein, genistein | Soy |
| FLAVANONES | Hesperetin, naringenin | Citrus fruit, tomatoes |
| ANTHOCYANIDINS | Pelargonidin, | Berry fruits, red wine |
| | cyanidine, malvidin | |

Other micronutrients having potential utility in relation to protection or treatment of age-related diseases, and described by Mecocci et al include:
Non-flavonoid polyphenols: resveratrol and curcumin,
Carotenoids: lycopene, lutein, zeaxanthin, B-cryptoxanthin, a-carotene, and the most prominent carotenoid, β-carotene,
Crocin (the main chemical compound identified in saffron),
Diterpenes: for example, carnosic and rosmarinic acids are two of the most important antioxidant compounds in rosemary.

### Herbs and Plant Extracts

In addition to the plants described or cross-referenced above in relation to micronutrients and antioxidants, other plant extracts and herbs are reported to have benefit in CNS disorders—see Kumar, Vikas. “Potential medicinal plants for CNS disorders: an overview.” Phytotherapy Research 20.12 (2006): 1023-1035. These include Ginkgo biloba, Hypericum perforatum (St John's wort), Piper methysticum Forst. (Family Piperaceae) also called kava kava, Valeriana officinalis L. (Valerian), Bacopa monniera (which in India is locally known as Brahmi or Jalanimba), Convolvulus pluricaulis (also known as Shankhpushpi or shankapushpi)

### Oils and Fats

It is reported that ω-3 poyunsaturated fatty acid (PUFA), for example, may be a promising tool for preventing age-related brain deterioration. Sources of PUFA such as (docosahexaenoic acid (DHA, 22:6) and eicosapentenoic acid (EPA, 20:5) include fish oils (Denis, I., et al. “Omega-3 fatty acids and brain resistance to ageing and stress: body of evidence and possible mechanisms.” Ageing Research Reviews 12.2 (2013): 579-594.)

### Immediate Release Dosage Units

The formulations and compositions (especially pharmaceutical compositions) may be prepared to provide for rapid or slow release; immediate, delayed, timed, or sustained release; or a combination thereof.

An immediate release product allows the ingredient or active moiety to dissolve in the gastrointestinal tract, without causing any delay or prolongation of the dissolution or absorption of the drug. Requirements for dissolution testing of immediate release products are set out in the Guidance for Industry (CDER 1997) “Dissolution testing for immediate release solid oral dosage forms”, (CDER 1997) “Immediate release solid oral dosage forms-Scale up and Post approval Changes”, ICH Guidance Q6A, Specifications: Test Procedures and Acceptance Criteria For New Drug Substances And New Drug Products. The most commonly employed dissolution test methods as described in the USP and European Pharmacopeia (6th edition) are the basket method (USP 1) and the paddle method (USP 2). The described methods are simple, robust, well standardized, and used worldwide. They are flexible enough to allow dissolution testing for a variety of drug products. The following parameters influencing the dissolution behaviour may for example be relevant for selecting the appropriate in vitro dissolution test conditions for an immediate release solid oral product: apparatus, stirring speed, dissolution medium and temperature. Because of the biopharmaceutical properties of MTC and its expected desirable absorption characteristics in the upper gastrointestinal tract, it was preferable to produce rapidly dissolving tablets of MTC.

Compositions according to the invention can be dissolution tested in a USP-2 apparatus in 900 ml of 0.1N HCl, with paddles rotating at 50-75 rpm. Compositions according to the invention exhibit at least the acceptance criteria cited for Stage 1 (S1) testing in the USP 32 (The United States Pharmacopeia, edited by the United States Pharmacopeial Convention, Inc., 12601 Twinbrook Parkway, Rockville, MD 20852; Published by Rand Mcnally, Inc., 32nd Edition, 2008):

Acceptance Criteria: Each tablet achieved 85% dissolution of MTC within 30 minutes after insertion of the coated tablet into the 0.1N HCl.

Thus in some embodiments, the MTC based formulations of the invention, when evaluated using this method, provide at least:
75% dissolution of MTC within 45 minutes after insertion of the coated tablet into the 0.1N HCl; or
85% dissolution of MTC within 30 minutes after insertion of the coated tablet into the 0.1N HCl;
85% dissolution of MTC within 15 minutes after insertion of the coated tablet into the 0.1N HCl.

Another aspect of the present invention pertains to methods of making a low dosage MT compound pharmaceutical composition comprising admixing at least one MT compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

In some embodiments, the pharmaceutically acceptable carrier, diluent, or excipient is or comprises one or both of a glyceride (e.g., Gelucire 44/14®; lauroyl macrogol-32 glycerides PhEur, USP) and colloidal silicon dioxide (e.g., 2% Aerosil 200®; Colliodal Silicon Dioxide PhEur, USP).

Preferably the pharmaceutical compositions comprising a compound of the invention, in solid dosage form. The composition preferably further comprises at least one diluent suitable for dry compression. The pharmaceutical composition is characterised in that the compound exists in a substantially stable form.

The pharmaceutical composition will generally also include a lubricant. Examples of lubricants include magnesium stearate, calcium stearate, sodium stearyl fumarate, stearic acid, glycerylbehaptate, polyethylene glycol, ethylene oxide polymers (for example, those available under the registered trademark Carbowax from Union Carbide, Inc., Danbury, CT), sodium lauryl sulphate, magnesium lauryl stearate, mixtures of magnesium stearate with sodium lauryl sulphate, and hydrogenated vegetable oil. Preferred lubricants include calcium stearate, magnesium stearate and sodium stearyl fumarate. Most preferred as the lubricant is magnesium stearate. Lubricants generally comprise from about 0.5 to about 5.0% of the total (uncoated) tablet weight. The amount of lubricant employed is generally from about 1.0 to about 2.0%, preferably 0.5 to 2.0% w/w.

In addition to the diluent(s) and lubricant(s), other conventional excipients may also be present in the pharmaceutical compositions of the invention. Such additional excipients include disintegrants, binders, flavouring agents, colours and glidants. Some excipients can serve multiple functions, for example as both binder and tablet disintegrant.

A tablet disintegrant may be present in an amount necessary to achieve rapid dissolution. Disintegrants are excipients which oppose the physical forces of particle bonding in a tablet or capsule when the dosage form is placed in an aqueous environment. Examples of disintegrants include crosslinked polyvinylpyrrolidone (crospovidone), sodium starch glycolate, crosslinked sodium carboxymethyl cellulose (sodium croscarmellose), and pregelatinized starch. Generally the amount of disintegrant can be from 0 to about 25% w/w, more commonly from about 1% to about 15% w/w, and usually less than 10% or less than 5% w/w, of the composition.

Binders are excipients which contribute to particle adhesion in a solid formulation. Examples of binders include cellulose derivatives (carboxymethylcellulose, hydroxypropyl methylcellulose, hydroxypropyl cellulose, hydroxyethylcellulose, ethylcellulose, microcrystalline cellulose) and sugars such as lactose, sucrose, dextrose, glucose, maltodextrin, and mannitol, xylitol, polymethacrylates, polyvinylpyrrolidone, sorbitol, pregelatinized starch, alginic acids, and salts thereof such as sodium alginate, magnesium aluminum silicate, polyethylene glycol, carrageenan and the like. Generally, the amount of binder can vary widely, e.g. from 0% to 95% w/w of the composition. As noted above, excipients may serve multiple functions. For instance, the tabletting diluent may also serve as a binder.

Glidants are substances added to a powder to improve its flowability. Examples of glidants include magnesium stearate, colloidal silicon dioxide (such as the grades sold as Aerosil), starch and talc. Glidants may be present in the pharmaceutical composition at a level of from 0 to about 5% w/w. Again, however, it should be noted that excipients may serve multiple functions. The lubricant, for example magnesium stearate, may also function as a glidant.

Examples of colours that may be incorporated into the pharmaceutical compositions of the invention include titanium dioxide and/or dyes suitable for food such as those known as FD&C dyes and natural colouring agents. A colouring agent is unlikely to be used in the powder mixture that is compressed in accordance with the aspects of the invention discussed above, but may form part of a coating applied to the composition, as described below, in which case the colouring agent may be present in the film coat in an amount up to about 2.0% w/w.

The tablet is desirably coated with a conventional film coating which imparts toughness, ease of swallowing, and an elegant appearance to the final product. Many polymeric film-coating materials are known in the art. A preferred film-coating material is hydroxypropylmethylcellulose (HPMC) or polyvinyl alcohol-part hydrolysed (PVA). HPMC and PVA may be obtained commercially, for example from Colorcon, in coating formulations containing excipients which serve as coating aids, under the registered trademark Opadry. Opadry formulations may also contain talc, polydextrose, triacetin, polyethyleneglycol, polysorbate 80, titanium dioxide, and one or more dyes or lakes. Other suitable film-forming polymers may also be used, including hydroxypropylcellulose, vinyl copolymers such as polyvinyl pyrollidone and polyvinyl acetate, and acrylate-methacrylate copolymers. Use of a film coating is beneficial for ease of handling and because a blue coloured uncoated core may stain the inside of the mouth during swallowing. Coating also improves light stability of the dosage form.

Coating of the tablets may conveniently be carried out using a conventional coating pan. In preferred embodiments of the process, the coating pan is pre-heated using heated inlet air until the exhaust temperature reaches 35°−55° C., more preferably 40-50° C. This may typically require application of heated inlet air at an inlet temperature of 45-75° C., preferably 50-65° C., for 10-15 minutes. The tablet cores containing the active ingredient (e.g. LMTM) are then added to the coating pan and the aqueous film coat applied. The spray rate is controlled such that the bed temperature is maintained at 38-48° C., more preferably 42-44° C., until the desired weight gain (coating weight) has been achieved.

### Subjects, Patients and Patient Groups

The subject/patient may be an animal, a mammal, a placental mammal, a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), a monotreme (e.g. platypus), an ape (e.g., gorilla, chimpanzee, orangutan, gibbon), or a human.

In preferred embodiments, the subject/patient is a human who has been diagnosed as having one of the cognitive or CNS disorders described herein, or (for prophylactic treatment) assessed as being susceptible to one of the neurodegenerative disorders of protein aggregation (e.g. cognitive or CNS disorder) described herein—for example based on familial or genetic or other data.

The patient may be an adult human, and the dosages described herein are premised on that basis (typical weight 50 to 70 kg). If desired, corresponding dosages may be utilised for subjects outside of this range by using a subject weight factor whereby the subject weight is divided by 60 kg to provide the multiplicative factor for that individual subject.

The low dosage treatments of the present invention increase the feasibility of purely prophylactic treatments, since the reduced concentration of active ingredients inevitably reduces risk of any adverse side effects (and increases the safety profile) and hence increases the risk/benefit ratio for such prophylactic treatments.

Thus, for example, for diagnosis of AD, and assessment of severity, the initial selection of a patient may involve any one or more of: rigorous evaluation by experienced clinician; exclusion of non-AD diagnosis as far as possible by supplementary laboratory and other investigations; objective evaluation of level of cognitive function using neuropathologically validated battery.

Diagnosis of AD and other disorders described herein can be performed by physicians by methods well known to those skilled in the art.

In some embodiments it is preferred that the subject or patient group, if they are being treated in respect of AD, is one who is not receiving treatment with any of: an acetylcholinesterase inhibitor or an N-methyl-D-aspartate receptor antagonist. Examples of acetylcholinesterase inhibitors include Donepezil (Aricept™), Rivastigmine (Exelon™) or Galantamine (Reminyl™). An examples of an NMDA receptor antagonist is Memantine (Ebixa™, Namenda™).

For example, the AD subject or patient group may be entirely naïve to these other treatments, and have not historically received one or both of them. For example, the AD subject or patient group may have historically received one or both of them, but ceased that medication at least 1, 2, 3, 4, 5, 6, 7 days, or 2, 3, 4, 5, 6, 7, 8, 12, or 16 weeks, or more preferably at least 1, 2, 3, 4, 5 or 6 months etc. prior to treatment with an MT compound according to the present invention.

The present inventors have found that certain patients appear to be particularly responsive to intermittent administration of low dose MT compounds.

As described in the examples herein, a subgroup of patients receiving 4 mg MTC twice weekly were found to have steady state HMT plasma levels (measured 1 hour post-dose) which were much higher than average.

Without wishing to be bound by theory, these patients may be particularly good at absorbing and/or accumulating HMT, for reasons which are as yet unclear. Demographic and genetic factors do not appear to be significant. Furthermore patients in this subgroup, i.e. those with higher steady-state HMT plasma levels, appear to be more responsive to the therapeutic effects of MT. For example, as explained in the Examples below, patients in the ‘high HMT plasma’ subgroup of the control arm experienced no decline, or above baseline improvement, in ADAS-cog11 assessment over 12 months.

Without wishing to be bound by theory, patients demonstrating above average steady state HMT plasma levels may therefore be particularly suitable for treatment using the methods of the present invention.

In some embodiments, therefore, the invention provides a method as described herein, wherein the subject has a steady state plasma level of MT of at least 0.1 ng/ml.

In some embodiments, therefore, the invention provides a method as described herein, wherein the subject has a steady state plasma level of MT of at least 0.23 ng/ml.

In some embodiments, therefore, the invention provides a method as described herein, wherein the subject has a steady state plasma level of MT of at least 0.25 ng/ml.

In some embodiments, therefore, the invention provides a method as described herein, wherein the subject has a steady state plasma level of MT of at least 0.6 ng/ml.

In some embodiments, the steady state plasma level is measured one hour after dosage with a MT compound.

In some embodiments, the steady state plasma level is measured one hour after dosage with between 0.2 and 4 mg MT.

Any aspect of the present invention may include the active step of selecting the AD subject or patient group according to these criteria.

For example, the methods of the invention may involve a patient selection step comprising:
administering to a subject a low dose of a MT-containing compound, for example a dose as described herein;
taking a blood sample after administration of said dose of the MT-containing compound;
measuring or estimating the MT plasma concentration for said subject;
using said measurement or estimate to determine whether or not to treat said patient with a treatment method of the invention.

For example, if the MT plasma concentration is higher than a threshold figure after a period of time, then this would indicate a higher likelihood of therapeutic benefit from the low frequency, low dosage treatment.

For example, the methods of the invention may involve a patient selection step comprising:
administering to a subject a low dose of a MT-containing compound, for example a dose providing about 4 mg MT to the subject;
taking a blood sample, preferably about one hour after administration of said dose of the MT-containing compound;
determining a steady state MT plasma concentration for said subject;
wherein a steady state MT plasma concentration higher than a threshold, as defined herein, indicates a higher likelihood of therapeutic benefit from the treatment.

In some embodiments, the invention provides a method as described herein, for example for the treatment of Alzheimer's disease, wherein the subject has a steady state plasma level of MT of at least 0.1 ng/ml.

In some embodiments, the invention provides a method as described herein, for example for the treatment of mild cognitive impairment (MCI), wherein the subject has a steady state plasma level of MT of at least 0.23 ng/ml.

### Labels, Instructions and Kits of Parts

The unit dosage compositions described herein (e.g. a low dose MT containing compound plus optionally other ingredients, or MT composition more generally for treatment in AD) may be provided in a labelled packet along with instructions for their use.

In one embodiment, the pack is a bottle, such as are well known in the pharmaceutical art. A typical bottle may be made from pharmacopoeial grade HDPE (High-Density Polyethylene) with a childproof, HDPE pushlock closure and contain silica gel desiccant, which is present in sachets or canisters. The bottle itself may comprise a label and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding the neurodegenerative disorders of protein aggregation (e.g. cognitive or CNS disorder) for which the medication is intended.

Where the medication is indicated for AD, said label or instructions may provide information instructing the user that the compositions should not be used in conjunction with any of: an acetylcholinesterase inhibitor or an N-methyl-D-aspartate receptor antagonist.

Said label or instructions may provide information regarding the maximum permitted daily dosage of the compositions as described herein.

Said label or instructions may provide information regarding the suggested dosage regimen for the treatment, as described herein. For example, a suggested dosage frequency of every other day, twice weekly, once weekly, etc. The disclosure of dosage frequencies above thus applies mutatis mutandis to this aspect.

Said label or instructions may provide information regarding the suggested duration of treatment, as described herein. The disclosure of treatment duration above thus applies mutatis mutandis to this aspect.

Reversing and/or Inhibiting the Aggregation of a Protein

One aspect of the invention is the use of an MT compound or composition as described herein, to regulate (e.g., to reverse and/or inhibit) the aggregation of a protein, for example, aggregation of a protein associated with a neurodegenerative disease and/or clinical dementia. The aggregation will be associated with a disease state as discussed below.

Similarly, one aspect of the invention pertains to a method of regulating (e.g., reversing and/or inhibiting) the aggregation of a protein in the brain of a mammal, which aggregation is associated with a disease state as described herein, the treatment comprising the step of administering to said mammal in need of said treatment, a prophylactically or therapeutically effective amount of an MT compound or composition as described herein, that is an inhibitor of said aggregation.

Disease conditions treatable via the present invention are discussed in more detail below.

### Methods of Treatment

Another aspect of the present invention, as explained above, pertains to a method of treatment comprising administering to a patient in need of treatment a prophylactically or therapeutically effective amount of a compound as described herein, preferably in the form of a pharmaceutical composition.

### Use in Methods of Therapy

Another aspect of the present invention pertains to a compound or composition as described herein, for use in a method of treatment (e.g., of a disease condition) of the human or animal body by therapy.

### Use in the Manufacture of Medicaments

Another aspect of the present invention pertains to use of an MT compound or composition as described herein, in the manufacture of a medicament for use in treatment (e.g., of a disease condition).

In some embodiments, the medicament is a composition e.g. a low-dose unit dose composition as described herein.

### Diseases of Protein Aggregation

The compounds and compositions of the present invention are useful in the treatment or prophylaxis of diseases of protein aggregation.

Thus, in some embodiments, the disease condition is a disease of protein aggregation, and, for example, the treatment is with an amount of a compound or composition as described herein, sufficient to inhibit the aggregation of the protein associated with said disease condition.

The following Table lists various disease-associated aggregating proteins and the corresponding neurodegenerative disease of protein aggregation. The use of the compounds and compositions of the invention in respect of these proteins or diseases is encompassed by the present invention.

Diseases of protein aggregation

| | | | | |
|---|---|---|---|---|
| | | | Fibril | |
| | | Aggregating | subunit | |
| | | domain and/or | size | |
| Protein | Disease | mutations | (kDa) | Reference |

Neurodegenerative disorders

| | | | | |
|---|---|---|---|---|
| Prion protein | Prion diseases | Inherited and | 27 | Prusiner (1998) |
| | | sporadic forms | | |
| | (CJD, nvCJD, Fatal | PrP-27-30; many | 27 | Prusiner (1998) |
| | familial insomnia, | mutations. | | |
| | Gerstmann-Straussler- | | | |
| | Scheinker syndrome, | | | |
| | Kuru) | | | |
| | | Fibrillogenic | | Gasset et al. (1992) |
| | | domains: 113- | | |
| | | 120, 178-191, | | |
| | | 202-218. | | |
| Tau protein | Alzheimer's disease, | Inherited and | 10-12 | Wischik et al. (1988) |
| | Down's syndrome, FTDP- | sporadic forms | | |
| | 17, CBD, post-encephalitic | | | |
| | parkinsonism, Pick's | | | |
| | disease, parkinsonism | | | |
| | with dementia complex of | | | |
| | Guam | | | |
| | | Truncated tau | 10-12 | Wischik et al. (1988) |
| | | (tubulin-binding | | |
| | | domain) 297-391. | | |
| | | Mutations in tau | | Hutton et al. (1998) |
| | | in FTDP-17. | | |
| | | Many mutations | | Czech et al. (2000) |
| | | in presenilin | | |
| | | proteins. | | |
| Amyloid | Alzheimer's disease, | Inherited and | 4 | Glenner & Wong, |
| β-protein | Down's syndrome | sporadic forms | | (1984) |
| | | Amyloid β- | 4 | Glenner & Wong, |
| | | protein; 1-42(3). | | (1984) |
| | | Mutations in APP | | Goate et al. (1991) |
| | | in rare families. | | |
| Huntingtin | Huntington's disease | N-termini of | 40 | DiFiglia et al. (1997) |
| | | protein with | | |
| | | expanded | | |
| | | glutamine | | |
| | | repeats. | | |
| Ataxin) | Spinocerebellar ataxias | Proteins with | | Paulson et al. (1999) |
| | (SCA1, 2, 3, 7) | expanded | | |
| | | glutamine | | |
| | | repeats. | | |
| Atrophin | Dentatorubropallidoluysian | Proteins with | | Paulson et al. (1999) |
| | atrophy (DRPLA) | expanded | | |
| | | glutamine | | |
| | | repeats. | | |
| Androgen | Spinal and bulbar | Proteins with | | Paulson et al. (1999) |
| receptor | muscular atrophy | expanded | | |
| | | glutamine | | |
| | | repeats. | | |
| Neuroserpin | Familial encephalopathy | Neuroserpin; | 57 | Davis et al. (1999) |
| | with neuronal inclusion | S49P, S52R. | | |
| | bodies (FENIB) | | | |
| α-Synuclein | Parkinson's disease, | Inherited and | 19 | Spillantini et al. |
| | dementia with Lewy | sporadic forms | | (1998) also |
| | bodies, multiple system | | | PCT/GB2007/001105 |
| | atrophy | | | |
| | | A53T, A30P in | | Polymeropoulos et |
| | | rare autosomal- | | al. (1997) |
| | | dominant PD | | |
| | | families. | | |
| TDP-43 | FTLD-TDP | Several TDP-43 | 10-43 | Mackenzie et al. |
| | | mutations | | (2010) |
| | Amyotrophic lateral | Several TDP-43 | 10-43 | Mackenzie et al. |
| | sclerosis | mutations | | (2010) |
| Cystatin C | Hereditary cerebral | Cystatin C less | 12-13 | Abrahamson et al. |
| | angiopathy (Icelandic) | 10 residues; | | (1992) |
| | | L68Q. | | |
| Superoxide | Amyotrophic lateral | SOD1 mutations. | 16 | Shibata et al. (1996) |
| dismutase 1 | sclerosis | | | |

As described in WO 02/055720, WO2007/110630, and WO2007/110627, diaminophenothiazines have utility in the inhibition of such protein aggregating diseases.

Thus it will be appreciated that, except where context requires otherwise, description of embodiments with respect to tau protein or tau-like proteins (e.g., MAP2; see below), should be taken as applying equally to the other proteins discussed herein (e.g., β-amyloid, synuclein, prion, etc.) or other proteins which may initiate or undergo a similar pathological aggregation by virtue of conformational change in a domain critical for propagation of the aggregation, or which imparts proteolytic stability to the aggregate thus formed (see, e.g., the article by Wischik et al. in “Neurobiology of Alzheimer's Disease”, 2nd Edition, 2000, Eds. Dawbarn, D. and Allen, S. J., The Molecular and Cellular Neurobiology Series, Bios Scientific Publishers, Oxford). All such proteins may be referred to herein as “aggregating disease proteins.”

Likewise, where mention is made herein of “tau-tau aggregation”, or the like, this may also be taken to be applicable to other “aggregating-protein aggregation”, such as β-amyloid aggregation, prion aggregation, synuclein aggregation, etc. The same applies for “tau proteolytic degradation” etc.

### Preferred Aggregating Disease Target Proteins

Preferred embodiments of the invention are based on tau protein. The term “tau protein,” as used herein, refers generally to any protein of the tau protein family. Tau proteins are characterised as being one among a larger number of protein families which co-purify with microtubules during repeated cycles of assembly and disassembly (see, e.g., Shelanski et al., 1973, Proc. Natl. Acad. Sci. USA, Vol. 70, pp. 765-768), and are known as microtubule-associated-proteins (MAPs). Members of the tau family share the common features of having a characteristic N-terminal segment, sequences of approximately 50 amino acids inserted in the N-terminal segment, which are developmentally regulated in the brain, a characteristic tandem repeat region consisting of 3 or 4 tandem repeats of 31-32 amino acids, and a C-terminal tail.

MAP2 is the predominant microtubule-associated protein in the somatodendritic compartment (see, e.g., Matus, A., in “Microtubules” [Hyams and Lloyd, Eds.] pp. 155-166, John Wiley and Sons, New York, USA). MAP2 isoforms are almost identical to tau protein in the tandem repeat region, but differ substantially both in the sequence and extent of the N-terminal domain (see, e.g., Kindler and Garner, 1994, Mol. Brain Res., Vol. 26, pp. 218-224). Nevertheless, aggregation in the tandem-repeat region is not selective for the tau repeat domain. Thus it will be appreciated that any discussion herein in relation to tau protein or tau-tau aggregation should be taken as relating also to tau-MAP2 aggregation, MAP2-MAP2 aggregation, and so on.

In some embodiments, the protein is tau protein.

In some embodiments, the protein is a synuclein, e.g., α- or β-synuclein.

In some embodiments, the protein is TDP-43.

TAR DNA-Binding Protein 43 (TDP-43) is a 414 amino acid protein encoded by TARDBP on chromosome 1p36.2. The protein is highly conserved, widely expressed, and predominantly localised to the nucleus but can shuttle between the nucleus and cytoplasm (Mackenzie et al 2010). It is involved in transcription and splicing regulation and may have roles in other processes, such as: microRNA processing, apoptosis, cell division, stabilisation of messenger RNA, regulation of neuronal plasticity and maintenance of dendritic integrity. Furthermore, since 2006 a substantial body of evidence has accumulated in support of the TDP-43 toxic gain of function hypothesis in amyotrophic lateral sclerosis (ALS). TDP-43 is an inherently aggregation-prone protein and aggregates formed in vitro are ultrastructurally similar to the TDP-43 deposits seen in degenerating neurones in ALS patients (Johnson et al 2009). Johnson et al (2008) showed that when TDP-43 is overexpressed in a yeast model only the aggregated form is toxic. Several in vitro studies have also shown that C-terminal fragments of TDP-43 are more likely than full-length TDP-43 to form insoluble cytoplasmic aggregates that become ubiquitinated, and toxic to cells (Arai et al 2010; Igaz et al 2009; Nonaka et al 2009; Zhang et al 2009). Though Nonaka et al (2009) suggested that these cytoplasmic aggregates bind the endogenous full-length protein depleting it from the nucleus, Zhang et al (2009) found retention of normal nuclear expression, suggesting a purely toxic effect for the aggregates. Yang et al (2010) have described the capture of full-length TDP-43 within aggregates of C- and N-terminal fragments of TDP-43 in NSC34 motor neurons in culture. Neurite outgrowth, impaired as a result of the presence of such truncated fragments, could be rescued by overexpression of the full-length protein. Although the role of neurite outgrowth in vivo has not been established, this model would support the suggestion made by Nonaka and colleagues for a role of TDP-43 aggregation in ALS pathogenesis.

Mutant TDP-43 expression in cell cultures has repeatedly been reported to result in increased generation of C-terminal fragments, with even greater cytoplasmic aggregation and toxic effects than the wild-type protein (Kabashi et al 2008; Sreedharan et al 2008; Johnson et al 2009; Nonaka et al 2009; Arai et al 2010; Barmarda et al 2010; Kabashi et al 2010).

Where the protein is tau protein, in some embodiments of the present invention, there is provided a method of inhibiting production of protein aggregates (e.g. in the form of paired helical filaments (PHFs), optionally in neurofibrillary tangles (NFTs) in the brain of a mammal, the treatment being as described above.

### Preferred Indications-Diseases of Protein Aggregation

In one embodiment the present invention is used for the treatment of Alzheimer's disease (AD)—for example mild, moderate or severe AD.

Notably it is not only Alzheimer's disease (AD) in which tau protein (and aberrant function or processing thereof) may play a role. The pathogenesis of neurodegenerative disorders such as Pick's disease and progressive supranuclear palsy (PSP) appears to correlate with an accumulation of pathological truncated tau aggregates in the dentate gyrus and stellate pyramidal cells of the neocortex, respectively. Other dementias include fronto-temporal dementia (FTD); FTD with parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD) and others (see, e.g., the article by Wischik et al. in “Neurobiology of Alzheimer's Disease”, 2nd Edition, 2000, Eds. Dawbarn, D. and Allen, S. J., The Molecular and Cellular Neurobiology Series, Bios Scientific Publishers, Oxford; especially Table 5.1). All of these diseases, which are characterized primarily or partially by abnormal tau aggregation, are referred to herein as “tauopathies”.

Thus, in some embodiments, the disease condition is a tauopathy. In some embodiments, the disease condition is a neurodegenerative tauopathy.

In some embodiments, the disease condition is selected from Alzheimer's disease (AD), Pick's disease, progressive supranuclear palsy (PSP), fronto temporal dementia (FTD), FTD with parkinsonism linked to chromosome 17 (FTDP 17), frontotemporal lobar degeneration (FTLD) syndromes; disinhibition-dementia-parkinsonism-amyotrophy complex (DDPAC), pallido-ponto-nigral degeneration (PPND), Guam-ALS syndrome, pallido nigro luysian degeneration (PNLD), cortico-basal degeneration (CBD), dementia with argyrophilic grains (AgD), dementia pugilistica (DP) or chronic traumatic encephalopathy (CTE), Down's syndrome (DS), dementia with Lewy bodies (DLB), subacute sclerosing panencephalitis (SSPE), MCI, Niemann-Pick disease, type C (NPC), Sanfilippo syndrome type B (mucopolysaccharidosis III B), or myotonic dystrophies (DM), DM1 or DM2, or chronic traumatic encephalopathy (CTE).

In some embodiments, the disease condition is a lysosomal storage disorder with tau pathology. NPC is caused by mutations in the gene NPC1, which affects cholesterol metabolism (Love et al 1995) and Sanfilippo syndrome type B is caused by a mutation in the gene NAGLU, in which there is lysosomal accumulation of heparin sulphate (Ohmi et al. 2009). In these lysosomal storage disorders, tau pathology is observed and its treatment may decrease the progression of the disease. Other lysosomal storage disorders may also be characterised by accumulation of tau.

Use of phenothiazine diaminium salts in the treatment of Parkinson's disease and MCI is described in more detail in PCT/GB2007/001105 and PCT/GB2008/002066.

In some embodiments, the disease condition is Parkinson's disease, MCI, or Alzheimer's disease.

In some embodiments, the disease condition is MCI or Alzheimer's disease.

In some embodiments, the disease condition is MCI.

In a further aspect of the present invention, a method of treating MCI in a subject is provided, which method comprises orally administering to said subject a methylthioninium (MT) containing compound, wherein said administration provides a daily dosage of about 21 mg to about 29 mg MT. For example, a daily dosage of about 21, 22, 23, 24, 25, 26, 27, 28, or 29 mg MT. The MT compound in this aspect is preferably an MT⁺ salt, most preferably methylthioninium chloride (MTC). In an embodiment, the method comprises administration of MTC at a total daily dosage of about 21, 22, 23, 24, 25, 26, 27, 28, or 29 mg.

In another aspect of the present invention, a method of treating MCI in a subject is provided, which method comprises orally administering to said subject a methylthioninium (MT) containing compound, wherein said administration is at a dosage frequency of less than once daily and wherein said administration provides an amount of MT to the subject that corresponds to an average of 21 to 29 mg MT per day. In an embodiment, the method comprises administration of a total daily dosage of about 21, 22, 23, 24, 25, 26, 27, 28, or 29 mg of MT. The MT compound in this aspect is preferably an MT⁺ salt, most preferably methylthioninium chloride (MTC). In an embodiment, the method comprises administration of MTC at a total daily dosage between 21 and 29 mg. In an embodiment, the method comprises administration of MTC at a total daily dosage of about 21, 22, 23, 24, 25, 26, 27, 28, or 29 mg.

Optionally, treating MCI according to methods of the invention comprises inhibiting decline, preventing an expected decline, or improving the condition. For example, in some embodiments, treatment of MCI may comprise improving cognitive ability or function in a subject.

In some embodiments, the disease condition is Huntington's disease or other polyglutamine disorder such as spinal bulbar muscular atrophy (or Kennedy disease), and dentatorubropallidoluysian atrophy and various spinocerebellar ataxias.

In some embodiments, the disease condition is an FTLD syndrome (which may for example be a tauopathy or TDP-43 proteinopathy, see below).

In some embodiments, the disease condition is PSP or ALS.

TDP-43 proteinopathies include amyotrophic lateral sclerosis (ALS; ALS-TDP) and frontotemporal lobar degeneration (FTLD-TDP).

The role of TDP-43 in neurodegeneration in ALS and other neurodegenerative disorders has been reviewed in several recent publications (Chen-Plotkin et al 2010; Gendron et al 2010; Geser et al 2010; Mackenzie et al 2010).

ALS is a neurodegenerative disease, characterised by progressive paralysis and muscle wasting, consequent on the degeneration of both upper and lower motor neurones in the primary motor cortex, brainstem and spinal cord. It is sometimes referred to as motor neuron disease (MND) but there are diseases other than ALS which affect either upper or lower motor neurons. A definite diagnosis requires both upper and lower motor neurone signs in the bulbar, arm and leg musculature with clear evidence of clinical progression that cannot be explained by any other disease process (Wijesekera and Leigh 2009).

Although the majority of cases are ALS-TDP, there are other cases where the pathological protein differs from TDP-43. Misfolded SOD1 is the pathological protein in ubiquitin-positive inclusions in ALS with SOD1 mutations (Seetharaman et al 2009) and in a very small subset (approximately 3-4%) of familial ALS, due to mutations in FUS (fused in sarcoma protein), the ubiquitinated pathological protein is FUS (Vance et al 2009; Blair et al 2010). FUS, like TDP-43, appears to be important in nuclear-cytoplasmic shuttling although the ways in which impaired nuclear import of FUS remains unclear. A new molecular classification of ALS, adapted from Mackenzie et al (2010), reflects the distinct underlying pathological mechanisms in the different subtypes (see Table below).

New Molecular Classification of ALS (modified from Mackenzie et al 2010). In the majority of cases, TDP-43 is the pathological ubiquitinated protein found in ALS.

Ubiquitin-positive inclusions in ALS

| Ubiquitinated disease protein | TDP-43 | FUS | SOD1 |
|---|---|---|---|
| Clinico-pathologic subtype | ALS-TDP | ALS-FUS | ALS-SOD1 |
| Associated genotype | TARDBP | FUS | SOD1 |
| Frequency of ALS cases | Common | Rare | Rare |

Amyotrophic lateral sclerosis has been recognised as a nosological entity for almost a century and a half and it is recognised in ICD-10 is classified as a subtype of MND in ICD 10 (G12.2). Reliable clinical diagnostic criteria are available for ALS, which differ little from Charcot's original description, and neuropathological criteria, reflecting the underlying molecular pathology, have also been agreed.

While ALS is classified pathologically into three subgroups, ALS-TDP, ALS-SOD1 and ALS-FUS, both latter conditions are rare. The largest study to date showed all sporadic ALS cases to have TDP-43 pathology (Mackenzie et al 2007). Only around 5% of ALS is familial (Byrne et al 2010) and mutations in SOD1, the commonest mutations found in FALS, account for between 12-23% of cases (Andersen et al 2006). SOD1 may also be implicated in 2-7% of SALS. Mutations in FUS appear to be far less common, accounting for only around 3-4% of FALS (Blair et al 2010). So it can be reliably predicted that a clinical case of SALS will have TDP-43 based pathology. Similarly this can be reliably predicted in FALS due to mutations in TDP-43, which account for around 4% of cases (Mackenzie et al 2010). ALS with mutations in: VCP, accounting for 1-2% of FALS (Johnson et al 2010), ANG (Seilhean et al 2009), and CHMP2B (Cox et al 2010) have also been reported to be associated with TDP-43 positive pathology. Although SOD1, FUS and ATXN2 mutations have not been found to be associated with TDP-43 positive aggregates, it has however been reported that TDP-43 is implicated in the pathological processes putatively arising from these mutations (Higashi et al 2010; Ling et al 2010; Elden et al 2010).

It is therefore established that TDP-43 has an important, and potentially central role, in the pathogenesis of the vast majority of SALS cases and may be implicated in the pathogenesis of a significant proportion of FALS. ALS is now widely considered to be a TDP-43 proteinopathy (Neumann et al 2009) and numerous in vitro, and in vivo studies provide support to the hypothesis that toxic gain of function, due to TDP-43 aggregation is responsible for at least some of the neurotoxicity in the disease.

FTLD syndromes are insidious onset, inexorably progressive, neurodegenerative conditions, with peak onset in late middle age. There is often a positive family history of similar disorders in a first degree relative.

Behavioural variant FTD is characterised by early prominent change in social and interpersonal function, often accompanied by repetitive behaviours and changes in eating pattern. In semantic dementia there are prominent word finding problems, despite otherwise fluent speech, with degraded object knowledge and impaired single word comprehension on cognitive assessment. Progressive non-fluent aphasia presents with a combination of motor speech problems and grammatical deficits. The core clinical diagnostic features for these three FTLD syndromes are shown in the Table below and the full criteria in Neary et al (1998).

### Clinical Profile and Core Diagnostic Features of FTLD Syndromes

| FTLD Syndrome -Clinical Profile | Core Diagnostic Features |
|---|---|
| Frontotemporal Dementia | 1. Insidious onset and gradual |
| Character change and disordered social | progression |
| conduct are the dominant features initially | 2. Early decline in social interpersonal |
| and throughout the disease course. | conduct |
| Instrumental functions of perception, | 3. Early impairment in regulation of |
| spatial skills, praxis and memory are intact | personal conduct |
| or relatively well preserved. | 4. Early emotional blunting |
| | 5. Early loss of insight |
| Semantic Dementia | A) Insidious onset and gradual progression |
| Semantic disorder (impaired | B) Language disorder characterised by |
| understanding of word meaning and/or | 1. Progressive, fluent empty speech |
| object identity) is the dominant feature | 2. Loss of word meaning manifest by |
| initially and throughout the disease course. | impaired naming and comprehension |
| Other aspects of cognition, including | 3. Semantic paraphasias and/or |
| autobiographic memory, are intact or | 4. Perceptual disorder characterised by |
| relatively well preserved. | 1. Prosopagnosia: impaired |
| | recognition of identity of familiar |
| | faces and/or |
| | 2. Associative agnosia: impaired |
| | recognition of object identity |
| | C) Preserved perceptual matching and |
| | drawing reproduction |
| | D) Preserved single word repetition |
| | E) Preserved ability to read aloud and |
| | write to dictation orthographically regular |
| | words |
| Progressive Non-fluent Aphasia | A) Insidious onset and gradual |
| Disorder of expressive language is the | progression |
| dominant feature initially and throughout | B) Non-fluent spontaneous speech with at |
| the disease course. Other aspects of | least one of the following: agrammatism, |
| cognition are intact or relatively well | phonemic paraphasias or anomia |
| preserved. | |

The discovery that TDP-43-positive inclusions characterize ALS and FTLD-TDP (Neumann et al 2006) was quickly followed by the identification of missense mutations in the TARDBP gene in both familial and sporadic cases of ALS (Gitcho of a/2008; Sreedharan et al., 2008). So far, 38 different TARDBP mutations have been reported in 79 genealogically unrelated families worldwide (Mackenzie et al 2010). TARDBP mutations account for approximately 4% of all familial and around 1.5% of sporadic ALS cases.

As of December 2010, mutations in thirteen genes which are associated with familial and sporadic ALS have been identified. Linkage of ALS to five other chromosome loci has been demonstrated but thus far specific mutations have not been identified.

### TDP-43 Proteinopathies

MT has a mode of action which targets and can reduce TDP-43 protein aggregation in cells, which is a pathological feature of the vast majority of both familial and sporadic ALS and is also characteristic of FTLD-P.

In addition laboratory data shows that methylthioninium inhibits the formation of TDP-43 aggregates in SH-SY5Y cells. Following treatment with 0.05 UM MT, the number of TDP-43 aggregates was reduced by 50%. These findings were confirmed by immunoblot analysis (Yamashita et al 2009).

The compounds and compositions of the invention may therefore be useful for the treatment of amyotrophic lateral sclerosis (ALS) and frontotemporal lobar degeneration (FTLD).

### Huntington's Disease and Polyglutamine Disorders

MT can reduce polyglutamine protein aggregation in cells, which is a pathological feature of Huntington's disease. Huntington's disease is caused by expansion of a translated CAG repeat located in the N-terminus of huntingtin. Wild-type chromosomes contain 6-34 repeats whereas, in Huntington's disease, chromosomes contain 36-121 repeats. The age of onset of disease correlates inversely with the length of the CAG tracts that code for polyglutamine repeats within the protein.

Laboratory data shows that methylthioninium inhibits the formation of aggregates of a huntingtin derivative containing a polyglutamine stretch of 102 residues in zebrafish (van Bebber et al. 2010). MT, when tested at 0, 10 and 100 μM, prevented the formation of such aggregates in zebrafish in a dose dependent manner. The compounds and compositions of the invention may therefore be useful for the treatment of Huntington's disease and other polyglutamine disorders such as spinal bulbar muscular atrophy (or Kennedy disease), and dentatorubropallidoluysian atrophy and various spinocerebellar ataxias (Orr & Zoghbi, 2007).

### Mitochondrial Diseases and Lafora Disease

The organ most frequently affected in mitochondrial disorders, particularly respiratory chain diseases (RCDs), in addition to the skeletal muscle, is the central nervous system (CNS). CNS manifestations of RCDs comprise stroke-like episodes, epilepsy, migraine, ataxia, spasticity, movement disorders, psychiatric disorders, cognitive decline, or even dementia (mitochondrial dementia). So far mitochondrial dementia has been reported in MELAS, MERRF, LHON, CPEO, KSS, MNGIE, NARP, Leigh syndrome, and Alpers-Huttenlocher disease (Finsterer, 2009). There are four complexes in the mitochondrial respiration chain, involving a series of electron transfers. Abnormal function of any of these complexes can result in mitochondrial diseases secondary to an abnormal electron transport chain and subsequent abnormal mitochondrial respiration. Complex III of the mitochondrial respiration chain acts to transfer electrons to cytochrome c.

Compounds and compositions of the invention may also be used to treat mitochondrial diseases which are associated with a deficient and/or impaired complex Ill function of the respiration chain. The compounds have the ability to act as effective electron carrier and/or transfer, as the thioninium moiety has a low redox potential converting between the oxidised and reduced form. In the event of an impaired and/or deficient function of Complex III leading to mitochondrial diseases, compounds of the invention are also able to perform the electron transportation and transfer role of complex III because of the ability of the thioninium moiety to shuttle between the oxidised and reduced form, thus acting as an electron carrier in place of sub-optimally functioning complex III, transferring electrons to cytochrome c.

Compounds and compositions of the invention also have the ability to generate an active thioninium moiety that has the ability to divert misfolded protein/amino acid monomers/oligomers away from the Hsp70 ADP-associated protein accumulation and/or refolding pathways, and instead rechannel these abnormal folded protein monomers/oligomers to the pathway that leads directly to the Hsp70 ATP-dependent ubiquitin-proteasome system (UPS), a pathway which removes these misfolded proteins/amino acid monomers/oligomers via the direct route (Jinwal et al. 2009).

Lafora disease (LD) is an autosomal recessive teenage-onset fatal epilepsy associated with a gradual accumulation of poorly branched and insoluble glycogen, termed polyglucosan, in many tissues. In the brain, polyglucosan bodies, or Lafora bodies, form in neurons. Inhibition of Hsp70 ATPase by MT (Jinwal et al. 2009) may upregulate the removal of misfolded proteins. Lafora disease is primarily due to a lysosomal ubiquitin-proteasomal system (UPS) defect because of a mutation in either the Laforin or Malin genes, both located on Chromosome 6, which result in inclusions that may accelerate the aggregation of misfolded tau protein. Secondary mitochondrial damage from the impaired UPS may further result in a suppressed mitochondrial activity and impaired electron transport chain leading to further lipofuscin and initiating the seizures that are characteristic of Lafora disease.

The MT moiety may disaggregate existing tau aggregates, reduce more tau accumulating and enhance lysosomal efficiency by inhibiting Hsp70 ATPase. MT may lead to a reduction in tau tangles by enhancing the ubiquitin proteasomal system removal of tau monomers/oligomers, through its inhibitory action on Hsp70 ATPase.

Thus compounds and compositions of the present invention may have utility in the treatment of Lafora disease.

### Mixtures of Oxidised and Reduced MT Compounds

MT compounds for use in the present invention may include mixtures of the oxidised and reduced form.

In particular, the LMT-containing compounds may include oxidised (MT⁺) compounds as ‘impurities’ during synthesis, and may also oxidize (e.g., autoxidize) after synthesis to give the corresponding oxidized forms. Thus, it is likely, if not inevitable, that compositions comprising the compounds of the present invention will contain, as an impurity, at least some of the corresponding oxidized compound. For example an “LMT” salt may include 10 to 15% of MT⁺ salt.

When using mixed MT compounds the MT dose can be readily calculated using the molecular weight factors of the compounds present.

### Salts and Solvates

Although the MT containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Clinical trial design. ‘Placebo’ contained 4 mg MTC tablets administered on a varying schedule on average 2 tablets per week to maintain the blind. MTC tablets were always given with the evening dose. MTC at low dose and frequency was chosen for control dosage, rather than LMTM, based on Phase 2 data showing that the minimal effective dose of MTC was 138 mg/day, with minimal efficacy at 69 mg/day.

FIG. 2. Least squares estimates of change from baseline over 12m in (A) ADAS-cog11 (cognitive) and (B) ADCS-ADL₂₃ (functional).

FIG. 3. Least squares estimates of change in whole brain volume (WBV; cm³) over 12m for control (plac) and 16 mg/day arms.

FIG. 4. Distribution of HMT concentrations (ng/ml) in control arm at steady state (blood levels at 1 hr post-dose) at first dose (Visit 2), 4 weeks (Visit 3) and 12 months (Visit 7). All patients received 16 mg/day (i.e. 8 mg twice daily) at Visit 7.

FIG. 5. Comparison of pre-dose and 1 hr plasma levels at Visits 2 & 3. Plasma MT concentration (ng/ml) one hour after dose (y-axis) vs. plasma MT concentration (ng/ml) pre-dose (x-axis).

FIG. 6. Distribution of plasma concentrations within control group, 1h post-dose Visit 3 (4 weeks). Patient density (%) vs plasma concentration (ng/ml). Majority of patients have measurable levels of HMT in plasma.

FIG. 7. Exposure-response in clinical trial, control arm. Plasma HMT concentration (ng/ml) vs ADAS-cog11 decline over 12 months for (A) all control group patients (B) all control group patients separated into MCI and AD sub-groups.

FIG. 8. Exposure-response for all clinical trial data (all doses pooled). Plasma HMT concentration (ng/ml) vs ADAS-cog11 decline over 12 months for (A) all patients in all arms of trial (B) all trial patients, separated into MCI and AD sub-groups.

FIG. 9. (A) Expected placebo decline in mild/moderate AD over 12 months from published studies: metadata analysis of expected decline data from published studies for ADAS-cog (4,357 patients) and ADCS-ADL (1,296 patients); (B) Comparison with expected placebo decline in mild AD over 12 months from published studies for (i) ADAS-cog (2,796 patients) and (ii) ADCS-ADL (1,259 patients).

FIG. 10. (A) Change in ADAS-cog11 plotted against parent MT levels (AD patients): prevention of cognitive decline in AD occurs from a threshold of 0.10 ng/ml (equivalent to 0.2 mg/day or 0.7 mg twice weekly). (B) Distribution of blood levels of MT in “placebo” arm of clinical trial; 89% of AD patients receiving MTC 4 mg, twice weekly had plasma levels above 0.10 ng/ml (Cₘₐₓ) after 12 months of treatment.

FIG. 11. (A) Change in ADAS-cog13 against parent MT levels (MCI patients): cognitive improvement in MCI over 12 months depends on exposure and is seen with HMTM 16 mg/day. Cognitive improvement in MCI required plasma levels >0.23 ng/ml. (B) Distribution of blood levels of MT; plasma levels >0.23 ng/ml were only seen in patients receiving HMTM 16 mg/day.

FIG. 12. Subjects receiving MTC 8 mg/week did not show brain atrophy or cognitive decline as expected for untreated placebo. (A) Change in Whole Brain Volume (WBV). There was no significant difference between trial subjects and matched Alzheimer's Neuroimaging Initiative (ADNI) group in respect of age, MMSE, treatment with AChEl/Memantine, education, smoking history, CDR-sob and APOE genotype and no difference in baseline brain atrophy (p=0.1104). (B) Change in ADAS-cog11 over 12 months, vs. a meta-analysis placebo (amyloid positive)-see FIG. 9. (C) Change in ADCS-ADL over 12 months, vs. a meta-analysis placebo (amyloid positive).

FIG. 13. Plasma concentration of HMT over 12 months.

### EXAMPLES

### Example 1—Provision of MT-Containing Compounds

Methods for the chemical synthesis of the MT-containing compounds described herein are known in the art. For example:

Synthesis of compounds 1 to 7 can be performed according to the methods described in WO2012/107706, or methods analogous to those.

Synthesis of compound 8 can be performed according to the methods described in WO2007/110627, or a method analogous to those.

Synthesis of compound 9 (MTC) is well known in the art. Examples syntheses of highly pure MTC are provided in WO2006/032879 and WO2008/007074.

Synthesis of compounds 10 to 13 can be performed according to the methods described in WO2007/110630, or methods analogous to those.

### Example 2—Formulation of MT-Containing Compounds

Methods for the chemical synthesis of the MT-containing compounds described herein are known in the art. Example methods using dry compression, for example, are provided in WO2012/072977.

### Example 3—Phase 3 Clinical Trial in Mild to Moderate AD

### Study Design

The LUCIDITY trial (NCT03446001; EudraCT: 2017-003558-17) is a Phase 3, randomized, double-blind, placebo-controlled, outpatient trial to evaluate the safety, efficacy, and tolerability of hydromethylthionine mesylate monotherapy in participants with severity ranging from mild cognitive impairment (MCI) to moderate AD. The initial 12-month blinded period is followed by a 12-month, open-label extension period to provide comparative, delayed-start data. There are 76 study sites located in Canada, France, Italy, Poland, Spain, United Kingdom and United States. The protocol was approved by an institutional review board or independent ethics committee at each site.

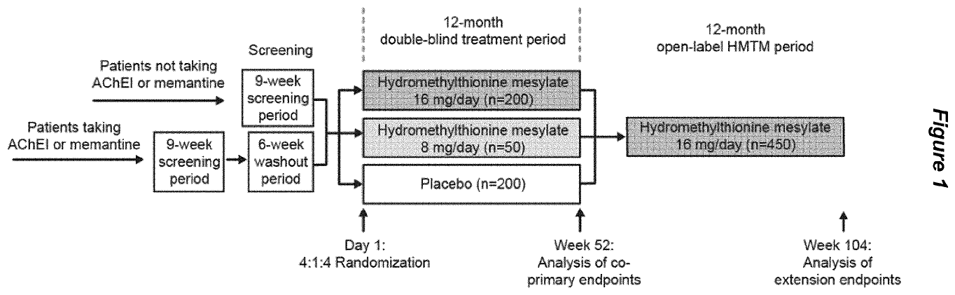

The study design is summarized in FIG. 1. Following screening, eligible participants are randomized at baseline in a 4:1:4 ratio to receive hydromethylthionine mesylate 16 mg/day, hydromethylthionine mesylate 8 mg/day, or placebo. Following completion of the 52-week double-blind treatment period, all participants continue open-label treatment with hydromethylthionine mesylate 16 mg/day for a further 52 weeks. Participants who were randomized to start on HMTM are considered early-starters; patients who begin on placebo and switch to HMTM at week 52 are considered delayed starters. Unlike a traditional delayed start design, in which participants remain blinded to treatment throughout the trial, treatment during weeks 52 to 104 is open-label, although participants and the site study staff remain blinded to prior treatment assignment. Randomization was stratified by severity (three levels: MMSE 16-19, 20-25, or 26-27, with a target ratio of approximately 2:3:1), prior use of symptomatic treatments (AChEls and/or memantine-two levels: prior use or none), and region (two levels: Canada/USA or UK/Europe). To achieve this target, enrolment was monitored and controlled at the site level for high recruiting sites and capped as needed at the study level. Patients who dropped out after randomization were not replaced, but participants were encouraged to continue with study visits off treatment until the scheduled completion of the double-blind treatment period (Visit 7). Only participants who continued in the study and receive hydromethylthionine mesylate treatment up to and including the last visit (Visit 10) without the addition of concomitant AChEls and/or memantine were eligible for continued treatment in a subsequent expanded access program.

### Study Drug and Placebo Formulation

The active and placebo treatment formulations are tablets that look visually the same. Hydromethylthionine mesylate can cause variable urinary discoloration. Therefore, to maintain blinding, the placebo group receives tablets containing a urinary discolourant (MTC, 4 mg) included among blank tablets containing only excipients on a varying schedule with an average frequency of 2/week.

Dosing frequency of 4 mg MTC tablets was on a varying schedule, such that each patient received a 4 mg tablet among the blanks on average twice per week. All subjects (on all arms) received 4 tablets at two times of day. This allowed for 2×4 mg followed by 2×4 mg to give 16 mg/day dose. 4 tablets were also given daily for placebo (2×placebo then 2×placebo) with 4 mg MTC tablets inserted at a rate of 2 per week i.e. 2 MTC tablets out of 28 per week, with remainder being true placebo.

### Inclusion and Exclusion Criteria

Participants had to be aged less than 90 years and meet the diagnostic criteria for probable AD or MCI-AD and must not have been taking an AChEl or memantine, for at least 60 days at Baseline. They must have been community-dwelling, have a mini mental state exam (MMSE) score of 16-27, and with functional impairment as evidenced by a Clinical Dementia Dating (CDR) stage of 0.5 to 2 at screening. Patients must also have had a positive amyloid PET scan. All patients must also have at least one study partner, consenting to their own participation; study partners can be changed so long as they have sufficient contact to complete outcome and safety assessments meaningfully and verify compliance with trial treatment.

Patients were excluded from the study if they have a substantial CNS cause for MCI or dementia other than AD, including significant vascular pathology seen on brain MRI. Other exclusions include severe, unstable, or poorly controlled medical or psychiatric conditions; pregnancy or breastfeeding: contra-indications or previous adverse reactions to MT or excipients; and involvement in another clinical trial or potential for lack of compliance as judged by the investigator. Stable doses of antipsychotics and antidepressants are permitted. Patients with glucose-6-phosphate dehydrogenase (G6PD) deficiency or taking medications with warnings or cautions about methaemoglobinemia are also excluded. Other pharmacologic agents that could affect cognition or pose undue risk are also excluded.

### Recruitment and Consent Procedures

Patients were recruited from memory clinics, outpatient clinics, or other components of specialist neurology, psychiatric, or geriatric medicine services. Where possible, fully informed, written consent was obtained from the patient. If the patient had reduced decision-making capacity, agreement to participate in the study is obtained to the patient's best level of understanding, supported by consent on the patient's behalf by a legally authorized representative.

### Assessments

The screening period is up to 9 weeks for participants who are not receiving an AChEl and/or memantine. For participants on AChEl and/or memantine who agree to discontinue, it may be extended for up to a further 6 weeks to allow for wash out. Eight post-Baseline visits are scheduled: five during the double-blind treatment period (Visit 3 for safety assessment; and Visits 4, 5, 6, and 7 at intervals of 3 months for assessments of efficacy, safety, and MRI) and three during the open-label phase. Visit 8, 4 weeks after commencing the open-label phase, is to assess safety; Visits 9 and 10 at intervals of 6 months are for efficacy and safety assessments, with brain imaging only at Visit 10 (Table 1).

At Visits 2, 3, 7 and 10, timed morning blood samples are collected for determination of plasma level of the drug. Samples are collected prior to dosing and then at 1, 2, and 4 hours post dose. A single blood sample for apolipoprotein E (ApoE) is obtained from participants who provide informed consent at any time after eligibility determination and prior to Visit 7. Blood may also be analyzed for other biomarkers for possible future research related to determination of potential biomarker predictors or surrogates for treatment response, to be described in a separate protocol.

### Safety and Tolerability

All safety assessments are performed during Screening to assess subject eligibility, by an independent qualified medical assessor not involved in efficacy assessments. For the enrolled participants, safety assessments are undertaken at Baseline and at each clinic visit after 4, 13, 26, 39, and 52 weeks during the double-blind treatment period, as well as after 56 (telephone assessment, or on-site in UK), 78, and 104 weeks in the open-label, delayed-start phase; when needed to follow up on a treatment-emergent adverse event (AE); and upon early termination (Table 1). Patients are followed as needed for the resolution or stabilization of any AE, consistent with the investigator's medical judgement.

### Primary Efficacy End Points

The co-primary end points of the LUCIDITY trial will be assessed in participants taking 16 mg/day hydromethylthionine mesylate, and compared with participants taking placebo. The co-primary end points are change from baseline to Week 52 in cognitive function measured by ADAS-Cog11, and functional abilities measured by ADCS-ADL₂₃.

### Secondary Efficacy End Points

Secondary end points to be assessed in participants taking placebo compared to participants taking 8 mg/day hydromethylthionine mesylate include:
1. Change from baseline to Week 52 in cognitive function measured by ADAS-Cog11, and functional abilities measured by ADCS-ADL₂₃.

Secondary end points to be assessed in participants taking placebo compared separately to participants taking 8 mg/day or 16 mg/day hydromethylthionine mesylate include change from baseline to Week 52 in:
1. Cognitive and functional abilities, measured by MMSE and CDR
2. Whole brain, parietal, and temporal lobe volume, measured by MRI; the annualized rate of atrophy from baseline to Week 52 will be quantified using the Boundary Shift Integral (BSI)
3. Brain metabolic function, measured by 18F-fluorodeoxyglucose positron emission tomography (18F-FDG-PET) change in temporal lobe Standardized Uptake Value Ratio (SUVR) (normalized to pons); this analysis will be restricted to participants with a CDR score of 0.5 at Screening, if a predefined threshold is reached for a sufficient number of participants providing data

Secondary end points to be assessed over the open-label delayed-start period (Week 52 to Week 104) will compare participants originally randomized to placebo (delayed starters) with participants originally randomized to either dose of hydromethylthionine mesylate (early starters) include:
4. Cognitive function measured by change from Week 52 to Week 104 in ADAS-Cog11.

### Exploratory End Points

1. TauRx Composite Scale is a new composite designed to be sensitive to decline in early AD constructed on the basis of data available from completed TauRx Phase 3 trials and from Alzheimer's Disease Neuroimaging Initiative data. The scale consists of cognitive subdomains (orientation, constructional praxis, word recall, assessor's rating of subject's speech, and assessor's rating of subject's comprehension) from the standard ADAS-Cog13, and functional items (use of telephone, keeping appointments, cooking and preparation of meals, and cleaning dishes) from the ADCS-ADL₂₃. Scores range from 0-48, with lower scores indicating greater impairment.
2. Change from Week 52 to Week 104 in ADCS-ADL₂₃, brain atrophy (MRI), and brain metabolic function (18F-FDG-PET), comparing delayed starters with early starters.
3. APOE genotype influence on primary and secondary outcomes.
4. Population pharmacokinetic (PK) analyses will be performed to estimate exposure in each subject for use in the evaluation of exposure-response relationships.

### Statistical Analysis

Sample size estimations to achieve 90% power (two-sided alpha=0.05) to detect a difference between hydromethylthionine mesylate 16 mg/day and placebo, the primary treatment group comparison in the double-blind treatment period, have been performed for the two co-primary clinical end points, assuming a withdrawal rate of 20% to 25%. The study sample size of approximately 450 is based on the ADCS-ADL₂₃, which has a larger standard deviation (SD) than ADAS-Cog11. Based on an estimated decline in ADCS-ADL₂₃ over 52 weeks in the control arm of 7.7 units with an estimated SD of 8.5 units, the study will have >90% power to detect a reduction in decline of 3.4 units or more. The 3.4-unit effect size is derived from an estimated treatment effect of 5.0±1.6 (mean±standard error) units in the completed hydromethylthionine mesylate studies. Based on an estimated decline of 6.5 units in ADAS-Cog11 over 52 weeks with an estimated SD of 5.9 units, 200 participants per treatment arm provide >90% power to detect a reduction in decline of 2.6 units or more, provided by a conservative value of the estimated treatment effect based on pooled completed Phase 3 studies of −5.2±1.3 (mean±standard error) units in completed hydromethylthionine mesylate studies.

With 200 participants randomized per arm to the primary comparison in the double-blind treatment period, 160 to 170 participants per arm will enter the open-label, delayed-start treatment phase, assuming the 20%-25% dropout rates mentioned above. Assuming a further 10% dropout during the delayed-start phase, the key secondary analysis to demonstrate disease modification by comparing early to late starters using a noninferiority margin of −2 ADAS-Cog11 units has approximately 80% power.

The primary analysis will be performed using the intent-to-treat (ITT) and efficacy modified intent-to-treat (E-MITT) populations. The ITT population will include all randomised participants. The E-MITT population will include all randomized participants who took at least one dose of study drug and have a baseline and a valid post-baseline efficacy assessment. The global null hypotheses are as follows:
H01: There is no difference in the change in ADAS-Cogn between the hydromethylthionine mesylate 16 mg/day and placebo groups from baseline to Week 52.
H02: There is no difference in the change in ADCS-ADL₂₃ between the hydromethylthionine mesylate 16 mg/day and placebo groups from baseline to Week 52.

The global null versus alternative primary efficacy hypotheses is a Union-Intersection Test which requires both the co-primary end points to show statistical significance at the 5% two-sided level of significance, for the global null hypothesis to be rejected.

### Evolution of Protocol Design

The LUCIDITY trial protocol has had 3 major revisions motivated by changing regulatory expectations for AD therapies and emerging data. As initially conceived in 2017 (Version 1.0, August 2017), a limited study was intended to confirm pharmacological activity of the 8 mg/day dose over 6 months in a population of 180 participants meeting diagnostic criteria for early AD, using change in FDG-PET as the primary outcome. At that time, it was envisaged that clinical end points would be assessed in a later larger study in mild to moderate AD. The first revision was in response to draft guidances issued by the FDA in February 2018 and by the European Medicines Agency (EMA) in March 2018 (28,29) indicating that a single trial could form the basis for regulatory approval in early AD on the basis of a statistically significant benefit with respect to placebo on a single composite clinical outcome scale comprising cognitive and functional elements. Accordingly, the trial was enlarged to 375 participants and lengthened to 9 months, with the intention of using a composite scale developed by TauRx based on the items found to be most sensitive and discriminatory from ADAS-Cog11 and ADCS-ADL₂₃ scales using data from the completed trials. In light of the emerging exposure-response data summarized above, a dose of 16 mg/day was added to the design. Scientific advice from the EMA in May 2019 indicated that the scope of the approval would be restricted to early AD if the trial were successful. Since hydromethylthionine mesylate has clinically relevant pharmacological activity over AD severity ranging from early to moderate disease (21), the study was changed to a more conventional design with co-primary cognitive (ADAS-Cog11) and functional (ADCS-ADL₂₃) end points. This entailed a further enlargement to 450 participants in the amended version of the protocol and a longer duration of the double-blind, placebo-controlled treatment phase to 12 months to ensure adequate power. The primary focus of the study was also changed to the 16 mg/day dose. The basic structure of the final amended design (Version 5.0) agreed upon with the EMA was for a double-blind, placebo-controlled treatment period of 12 months, followed by a modified delayed-start open-label extension period of 12 months in which participants initially randomized to placebo were switched to 16 mg/day to approximate a delayed-start design to investigate disease-modifying potential. Further modifications were introduced in October 2020 to allow for the impact of COVID-19. The statistical analysis plan was adjusted to accommodate these changes, and the current version is described briefly above. The study was powered on the basis of the primary comparison, 16 mg/day and placebo. A small 8 mg/day arm was retained in a 1:4 ratio with 16 mg/day and placebo to provide a bridge to prior completed trials and to include participants randomised to earlier versions of the protocol receiving this lower dose.

Patient selection criteria for the completed trial included MMSE score 16-17, age <90, meeting diagnostic criteria for Mild Cognitive Impairment (MCI) due to AD or mild to moderate AD, having a positive amyloid-PET scan. Further details are provided in Wischik et al., 2022.

The study recruited 20% over target in April 2021. The target MMSE ratio was achieved. All participants were expected to have completed the blinded phase by the end of March 2022, and top-line results are due mid-2022. Currently, 20% of the participants have terminated early from the double-blind phase. Fewer than 3% of visits have been impacted by COVID-19, and this does not appear to have affected the validity of the trial. A sufficient number of participants with CDR 0.5 have 18F-FDG-PET data available to enable analysis of this end point.

### Results/Discussion

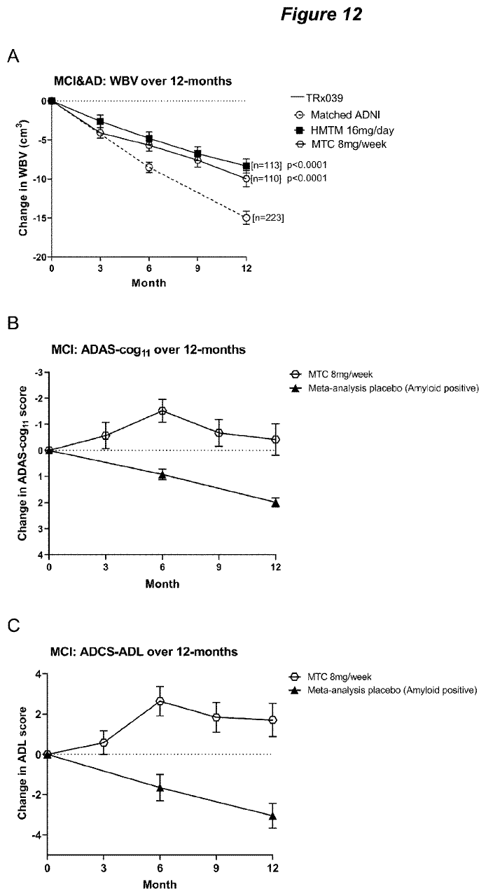

Because of the slight urinary discolouration caused by LMTM, participants in the control arm received a 4 mg dose of active drug taken on a varying schedule twice per week. This was needed to maintain study blindness in this placebo-controlled trial. The majority of participants on this arm declined as expected (i.e. predicted decline without active treatment, based on meta-analysis of data from previously published studies) over 52 weeks. However, a group from this arm benefited even with such a small dose of active drug (see FIG. 12, B, C).

Without wishing to be bound by theory, this may be attributable to accumulating low blood levels of drug in these participants, suggesting some people are very sensitive to LMTM. It appears that 4 mg MTC twice weekly leads to accumulation of MT in these patients, as further discussed below.

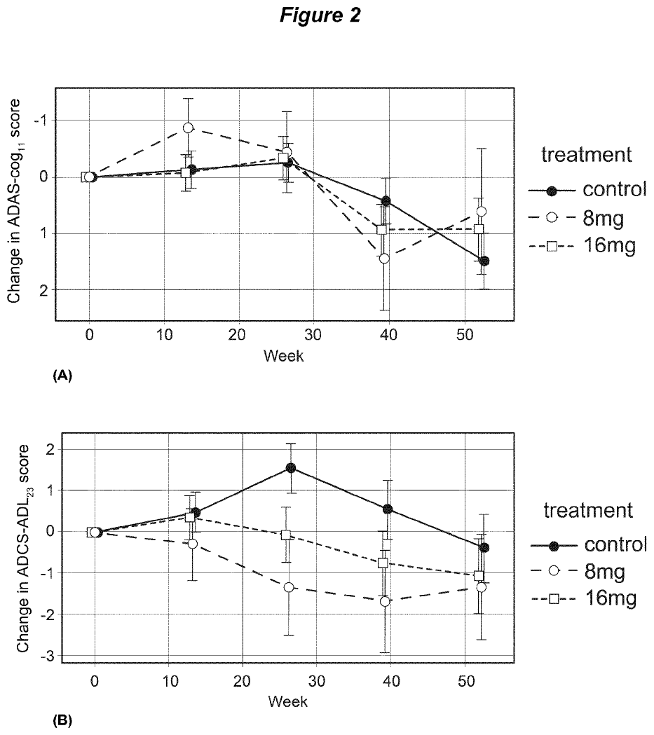

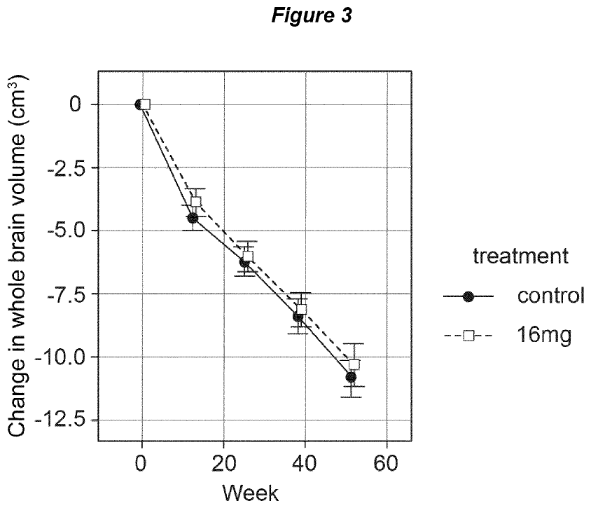

As shown in FIG. 2, for the primary efficacy endpoints (change in ADAS-cog11 and ADCS-ADL₂₃), results in the control arm are on average comparable to those in the treatment arms, with minimal or no decline over the duration of the trial. Changes in whole brain volume (WBV) were also equivalent (FIG. 3).

From the results available, a subset of patients with MCI appear to be particularly responsive to low dose MTC.

### Example 4—Analysis of Plasma HMT Levels

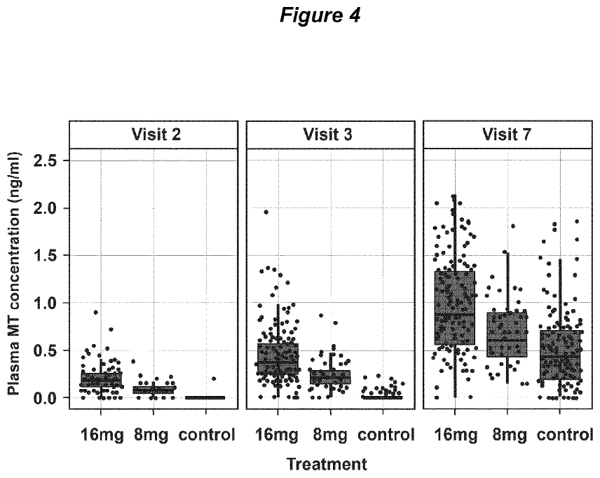

The distribution of HMT concentrations in the control group at steady state (blood levels at 1 hr post-dose) at first dose (Visit 2), 4 weeks (Visit 3) and 12 months (Visit 7) is shown in FIG. 4. Concentrations for the 8 mg LMTM/day and 16 mg LMTM/day are also shown. All patients received 16 mg/day (i.e. 8 mg twice daily) from Visit 7 onwards.

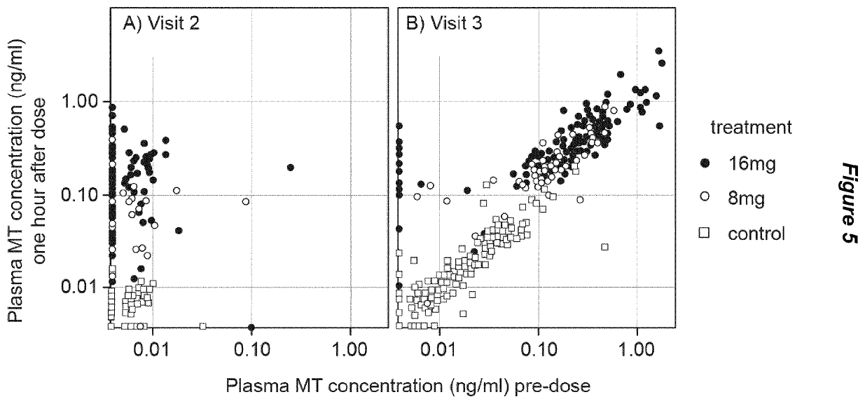

FIG. 5 shows the correlation between pre-dose and post-dose plasma levels at first dose (Visit 2) and 4 weeks treatment (Visit 3), across all three arms of the study.

Due to the dosage frequency in the control arm (4 mg MTC-containing capsules administered only twice weekly) the timing of the plasma measurement is a factor: notably, due to the trial design the placebo dose taken 1 hr before the measurement may or may not actually have contained MTC. Hence evidence of an accumulation of MT in some patients cannot be completely definitive. However, this fact alone cannot explain the distribution in plasma levels across the whole control group. Furthermore, although dosing the evening before may have improved cognitive scores the next day on 2/7 of visits randomly (since MTC is known to have nootropic properties at very low doses), this alone cannot explain the observed treatment effect in the control group.

The steady-state plasma concentrations (Visit 4. 1 h post-dose) in each dosing arm was investigated further. Patients were grouped based on plasma concentration. Mean values for each dosing arm and subgroup are shown in Table 2:

| | | Number of |
|---|---|---|
| | Mean value HMT conc (ng/ml) | patients |
| All dosing arms | 0.20 (SD = 0.33, SE = 0.02) | n = 457 |
| Concentration sub-group: | | |
| BLLOQ | 0.00 (SD = 0.00, SE = 0.00) | n = 71 |
| ultralow | 0.01 (SD = 0.01, SE = 0.00) | n = 114 |
| mid | 0.04 (SD = 0.01, SE = 0.00) | n = 51 |
| high | 0.41 (SD = 0.38, SE = 0.03) | n = 221 |

| | Dosing arm | | | | | |
|---|---|---|---|---|---|---|
| Conc | 16 mg | | 8 mg | | control | |
| sub | Mean HMT | Number of | Mean HMT | Number of | Mean HMT | Number of |
| group: | conc (ng/ml) | patients | conc (ng/ml) | patients | conc (ng/ml) | patients |
| BLLOQ | 0.00 | n = 3 | 0.00 | n = 3 | 0.00 | n = 65 |
| | (SD = 0.00, | | (SD = 0.00, | | (SD = 0.00, | |
| | SE = 0.00) | | SE = 0.00) | | SE = 0.00) | |
| ultralow | 0.02 | n = 3 | — | — | 0.01 | n = 111 |
| | (SD = 0.01, | | | | (SD = 0.01, | |
| | SE = 0.01) | | | | SE = 0.00) | |
| mid | 0.04 | n = 2 | 0.05 | n = 2 | 0.04 | n = 47 |
| | (SD = 0.00, | | (SD = 0.02, | | (SD = 0.01, | |
| | SE = 0.00) | | SE = 0.01) | | SE = 0.00) | |
| high | 0.50 | n = 146 | 0.26 | n = 59 | 0.12 | n = 16 |
| | (SD = 0.43, | | (SD = 0.16, | | (SD = 0.05, | |
| | SE = 0.04) | | SE = 0.02) | | SE = 0.01) | |

* note that values listed as BLLOQ (below limit of quantification) are set to zero

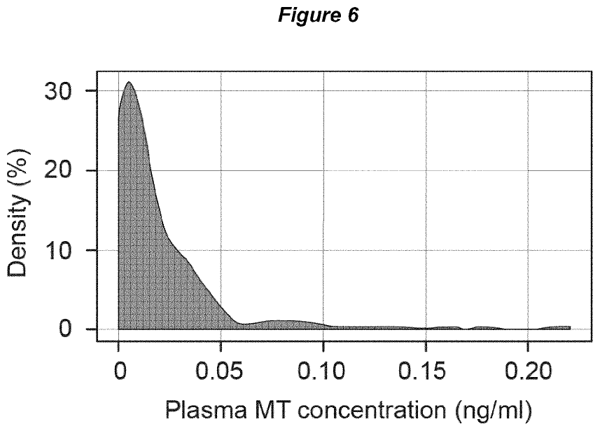

Further analysis of the plasma HMT levels within the control group is illustrated in FIG. 6 which shows the distribution of different steady state plasma concentrations within that group (Visit 4. 1 h post-dose).

In general, it can be seen that, as expected, HMT plasma levels in the 16 mg LMTM/day group are higher than in the control group. At week 4 the overall mean plasma concentration in the control group (1h post dose) is 0.12 ng/ml, compared to 0.50 ng/ml in the 16 mg LMTM/day group. However, some patients in the control group appear to have higher levels than others.

It appears that 4 mg MTC twice weekly leads to accumulation of MT in these patients, as illustrated in e.g. FIG. 5. This has the consequence that the relationship between dose and steady-state plasma levels becomes non-linear at these very low doses. This is unexpected as the half life of MT in this elderly population is approximately 22 hrs, therefore there should be negligible accumulation at a mean dosing interval of 84 hrs (i.e. twice weekly). As shown in FIG. 6, the majority of control patients have measurable levels of HMT in plasma. The overall mean HMT concentration in this group was approximately 0.012 ng/ml. It is likely that all MT⁺ would be converted to HMT at this very low dose. Food-dependent limitations in absorption appear only at much higher MTC doses (Baddeley et al., 2015).

There are 111 control group patients with plasma levels between 0.005-0.025 ng/ml (ultra-low), 47 between 0.025-0.06 ng/ml (mid) and 16 cases with >0.06 ng/ml (high). There are also 68 cases with plasma levels <0.005 ng/ml (undetectable).

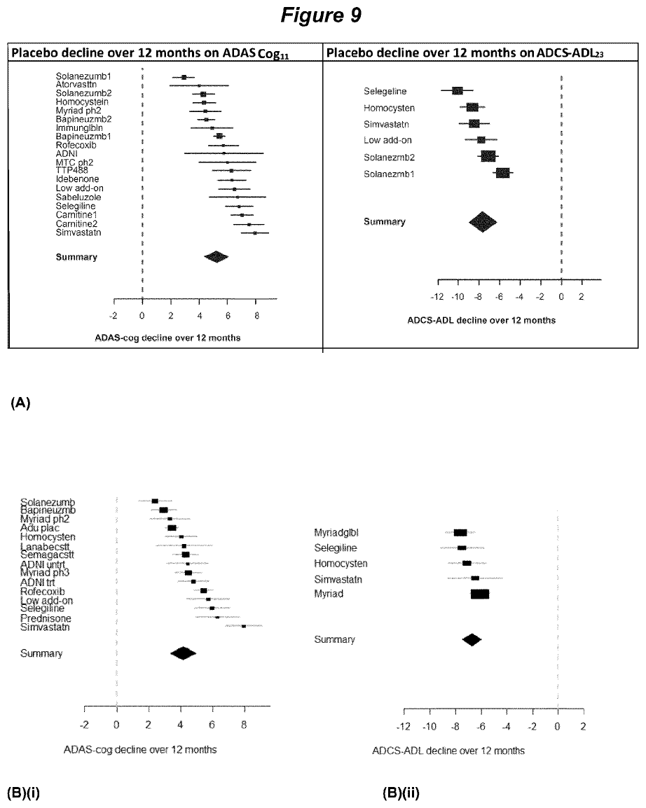

In the control group, the ultra-low cases have ADAS-cog decline as expected (3.75±0.79). The expected decline. i.e. the decline that would occur without active treatment, can be modelled, based on metadata analysis of expected decline data, from previously published studies (see, e.g. FIG. 9).

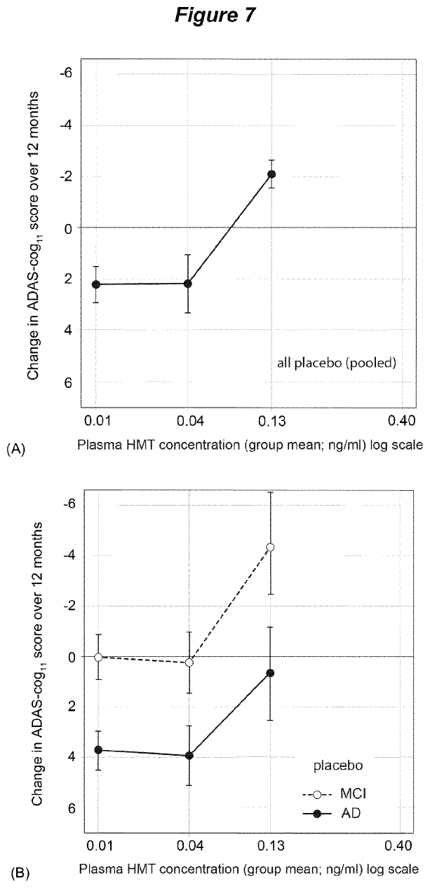

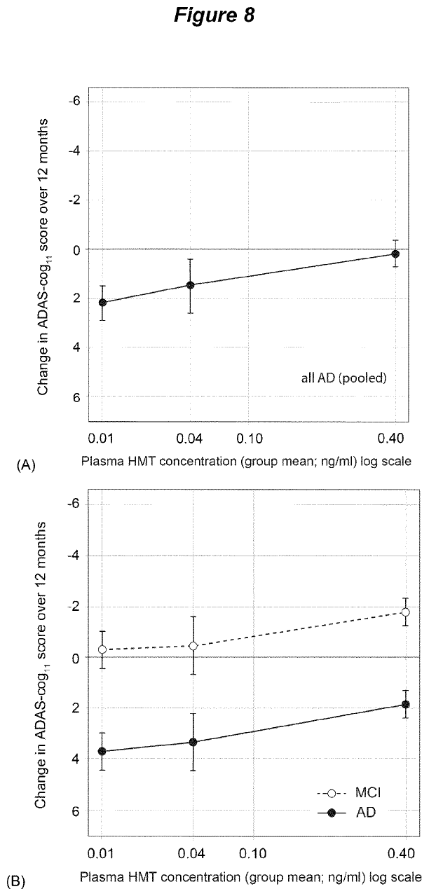

Compared to this expected decline, the treatment effect for the high group is-4.73±2.01, so zero decline or above baseline. Including everyone, the overall treatment effect at 16 mg/day relative to the control group with ultra-low plasma levels is-1.79 ADAS-cog units (p=0.0400). Exposure response as a function of mean plasma HMT levels in the three groups is shown in FIG. 7 (for control group). For comparison, exposure response across the whole study (all three treatment arms) is shown in FIG. 8.

### Example 5—Clinical Trial Results-Summary

The Phase 3 LUCIDITY study compared a dose of 16 mg/day with methylthioninium chloride (MTC) given at a dose of 4 mg twice weekly, the minimum required to prevent bias arising from potential urinary discolouration. The study was conducted in 598 patients with AD severity ranging from Mild Cognitive Impairment (MCI) through to the moderate stage of disease.

TauRx has now completed the first 12-month double-blind phase of the trial. This was followed by a further 12-month period, which is still ongoing, during which all patients receive HMTM 16 mg/day. All patients in this study were required to have a positive amyloid-PET scan and not taking standard symptomatic treatments for AD.

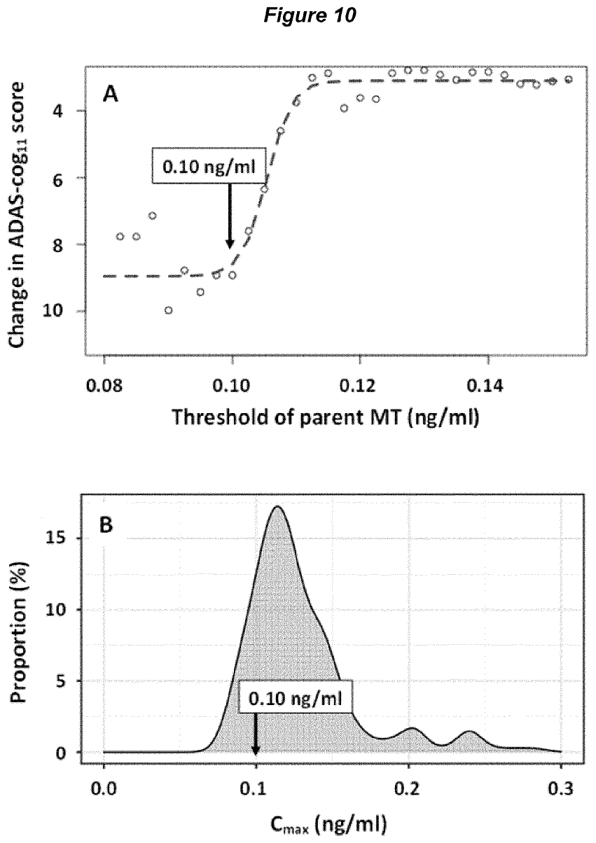

Of patients receiving MTC 4 mg twice weekly, the majority (85%) were unexpectedly found to have blood levels of active drug above the threshold needed to produce a clinical effect (see FIG. 10).

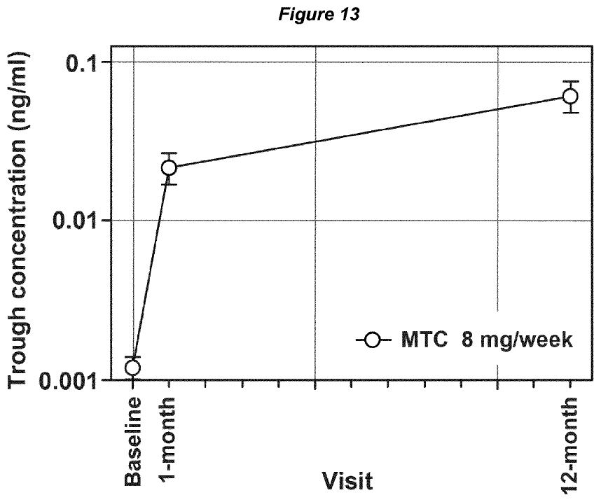

The PK of MTC/LMTM seems to show further accumulation from visit 3 (4 weeks) to visit 7 (12 months) regardless of the dose (a 2-fold increase is observed) (see FIG. 13). Trough levels were found to increase over time by 9-fold over predicted steady state. Interestingly, no corresponding increase in inactive glucuronide metabolite (which accounts for ˜99% of plasma total HMT/MT) was seen. Therefore there was no overall accumulation of HMT/MT, but there may have been time-dependent inhibition of the conversion of HMT to the glucuronide metabolite. Overall, the HMT accumulation from the MTC 8 mg/week dose is sufficient to produce a treatment effect.

In the absence of a true placebo, the trial as designed could not determine outcomes on primary clinical endpoints relative to a therapeutically inactive placebo as prespecified. In light of the evidence now available, TauRx does not believe that a blinded placebo-controlled trial using clinical endpoints is technically feasible. TauRx has therefore analysed the data in terms of the relationship between blood concentration and treatment effect, change from pre-treatment baseline, and comparisons against historical controls available from closely matched data from the Alzheimer's Neuroimaging Initiative (ADNI).

The overall baseline MMSE score was 21 for the study population spanning MCI through to moderate disease. There was minimal decline over the first 12 months in patients receiving the 16 mg/day dose on both of the coprimary cognitive and functional endpoints (1.3 ADAS-cog11 units and −1.0 ADCS-ADL₂₃ units). The expected decline in an untreated population would be approximately 5 units on both scales.

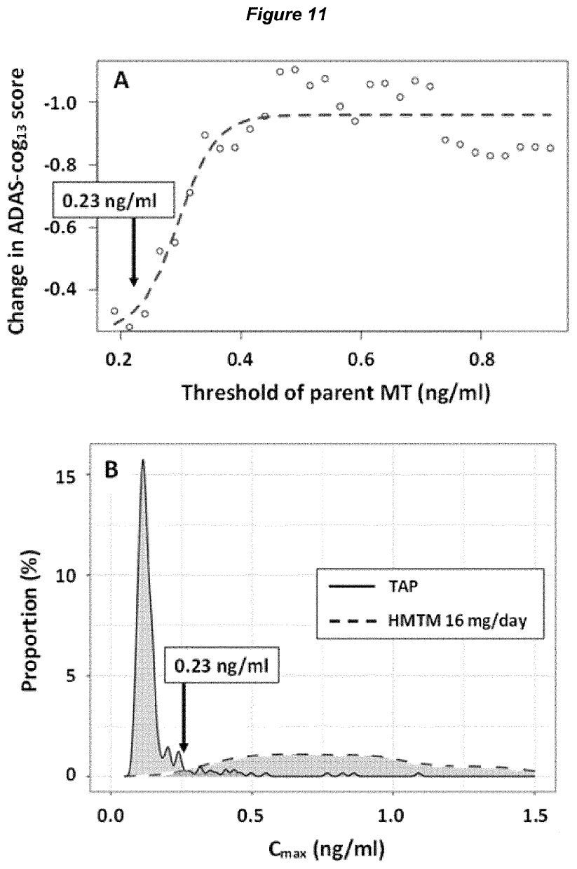

In the 105 patients with MCI (baseline MMSE score 23) receiving the 16 mg/day dose, there was statistically significant cognitive improvement of 2 units over baseline at 6 months (p=0.0002), 12 months (p=0.0391) and 18 months (p=0.0473) on the ADAS-cog13 scale. The plasma level threshold needed to produce this improvement was determined (FIG. 11). The mean change on the instrumental activities of daily living subscale of ADCS-ADL also remained above the pre-treatment baseline at 6, 12 and 18 months.

In the 147 patients with mild/moderate AD (baseline MMSE 20) receiving 16 mg/day, there was a 2.5 unit cognitive decline in the first 9 months and no further decline over the following 9 months. The functional decline on the ADCS-ADL scale was −2 units at 12 months and −3 units at 18 months representing a reduction in decline of about 75% relative to a published meta-analysis of publicly available placebo decline data from historical trials in mild to moderate AD.

Statistically significant reductions in disease progression as measured by change in cognitive function (p=0.0008) and brain atrophy (p<0.0001) were confirmed by comparisons of participants receiving the 16 mg/day dose against ADNI subjects who were closest to the study population by age and clinical severity (see FIG. 12A). The differences remained statistically significant in both MCI and AD subgroups. As expected, LUCIDITY trial participants with MCI entered the study with more brain atrophy than ADNI healthy aging subjects and consistent with ADNI MCI subjects. Those treated with HMTM 16 mg/day had a rate of progression of brain atrophy that was significantly less than in ADNI MCI subjects (p<0.0001) and comparable to that seen in ADNI healthy aging subjects.

Recent trials that have tested treatments targeting amyloid have been conducted in comparable or milder populations than the MCI group in the LUCIDITY trial. When HMTM is compared to publicly available placebo decline data from these studies as a benchmark, the treatment effects on cognitive and functional decline are about three-fold larger over 18 months. The benefit seen with HMTM is clinically meaningful for people with Alzheimer's.

The safety profile seen in LUCIDITY remains strong and consistent with earlier published HMTM trial data. There were no treatment-related serious adverse events or evidence of amyloid related imaging abnormalities (ARIA).

### REFERENCES

### References for Discussion of Background Art

Al-Hilaly Y K, Pollack S J, Rickard J E, Simpson M, Raulin A C, Baddeley T, Schellenberger P, Storey J M D, Harrington C R, Wischik C M, Serpell L C. Cysteine-Independent Inhibition of Alzheimer's Disease-like Paired Helical Filament Assembly by Leuco-Methylthioninium (LMT). J Mol Biol. 2018 Oct. 19; 430 (21): 4119-4131.
Alzheimer A. Über eine eigenartige Erkrankung der Hirnrinde. Allg Z Psych Psych-gerich Med 1907; 64:146-8 [German].
Alzheimer's Disease International. World Alzheimer Report 2015: The global impact of dementia, an analysis of prevalence, incidence, cost and trends. World Alzheimer Report 2015.
Arriagada P W, Growdon J H, Hedley-White E T, Hyman B T. Neurofibrillary tangles but not senile plaques parallel duration and severity of Alzheimer's disease. Neurology 1992; 42:631-9.
Baddeley T, C., McCaffrey J, Storey JMD, et al. Complex disposition of methylthioninium redox forms determines efficacy in tau aggregation inhibitor therapy for Alzheimer's disease. J Pharmacol Exptl Therapeutics 2015; 352:110-8.
Braak H, Del Tredici K. The pathological process underlying Alzheimer's disease in individuals under thirty. Acta Neuropathol 2011; 121:171-81.
Brier M R, Gordon B, Friedrichsen K, et al. Tau and Aβ imaging, CSF measures, and cognition in Alzheimer's disease. Science Transl Med 2016; 8: 338ra66.
DiSanto A R, Wagner J G. Pharmacokinetics of highly ionized drugs. II. Methylene blue-absorption, metabolism, and excretion in man and dog after oral administration. J Pharmaceut Sci 1972; 61:1086-90.
Gauthier, S., Feldman, H. H., Schneider, L. S., Wilcock, G. K., Frisoni, G. B., Hardlund, J. H., Moebius, H. J., Bentham, P., Kook, K. A., Wischik, D. J., Schelter, B. O., Davis, C. S., Staff, R. T., Bracoud, L., Shamsi, K., Storey, J. M. D., Harrington, C. R. & Wischik, C. M. (2016) Efficacy and safety of tau-aggregation inhibitor therapy in patients with mild or moderate Alzheimer's disease: a randomised, controlled, double-blind, parallel-arm, phase 3 trial. The Lancet 388, 2873-2884.
Geerts H, Spiros A, Roberts P, Carr R. A strategy for developing new treatment paradigms for neuropsychiatric and neurocognitive symptoms in Alzheimer's disease. Front Pharmacol 2013; 4:47.
Giannakopoulos P, Herrmann F R. Bussiere T, et al. Tangle and neuron numbers, but not amyloid load, predict cognitive status in Alzheimer's disease. Neurology 2003; 60:1495-500.
Harrington C R, Storey J M D. Clunas S, et al. Cellular models of aggregation-dependent template-directed proteolysis to characterize tau aggregation inhibitors for treatment of Alzheimer's disease. J Biol Chem 2015; 290:10862-75.
Huang Y, Mucke L. Alzheimer mechanisms and therapeutic strategies. Cell 2012; 148:1204-22.
Josephs K A, Whitwell J L, Ahmed Z, et al. b-Amyloid burden is not associated with rates of brain atrophy. Ann Neurol 2008; 63:204-12.
Lai R Y K, Harrington C R, Wischik C M. Absence of a role for phosphorylation in the tau pathology of Alzheimer's disease. Biomolecules 2016; 6:19.
Maruyama M, Shimada H, Suhara T, et al. Imaging of tau pathology in a tauopathy mouse model and in Alzheimer patients compared to normal controls. Neuron 2013; 79:1094-108.
May J M, Qu Z-c, Cobb C E. Reduction and uptake of methylene blue by human erythrocytes. Am J Physiol—Cell Physiol 2004; 286: C1390-C8.
Melis V, Magbagbeolu M, Rickard J E, et al. Effects of oxidized and reduced forms of methylthioninium in two transgenic mouse tauopathy models. Behav Pharmacol 2015; 26:353-68.
Mukaetova-Ladinska E B, Garcia-Sierra F, Hurt J, et al. Staging of cytoskeletal and b-amyloid changes in human isocortex reveals biphasic synaptic protein response during progression of Alzheimer's disease. Am J Pathol 2000; 157:623-36.
Mullane K, Williams M. Alzheimer's therapeutics: Continued clinical failures question the validity of the amyloid hypothesis-but what lies beyond? Biochem Pharmacol 2013; 85:289-305.
Peter C, Hongwan D, Kupfer A, Lauterburg B H. Pharmacokinetics and organ distribution of intravenous and oral methylene blue. Eur J Clin Pharmacol 2000; 56:247-50.
Schelter B O, Shiells H, Baddeley T C, Rubino C M, Ganesan H, Hammel J, Vuksanovic V, Staff R T, Murray A D, Bracoud L, Riedel G, Gauthier S, Jia J, Bentham P, Kook K, Storey J M D, Harrington C R, Wischik C M. Concentration-Dependent Activity of Hydromethylthionine on Cognitive Decline and Brain Atrophy in Mild to Moderate Alzheimer's Disease. J Alzheimers Dis. 2019; 72 (3): 931-946.
Schneider A, Biernat J, von Bergen M, Mandelkow E, Mandelkow E-M. Phosphorylation that detaches tau protein from microtubules (Ser262, Ser214) also protects it against aggregation into Alzheimer paired helical filaments. Biochemistry 1999; 38:3549-58.
Winblad B, Amouyel P, Andrieu S, et al. Defeating Alzheimer's disease and other dementias: a priority for European science and society. Lancet Neurol 2016; 15:455-532.
Wischik C M, Crowther R A, Stewart M, Roth M. Subunit structure of paired helical filaments in Alzheimer's disease. J Cell Biol 1985; 100:1905-12.
Wischik C M, Edwards P C, Lai R Y K, et al. Quantitative analysis of tau protein in paired helical filament preparations: implications for the role of tau protein phosphorylation in PHF assembly in Alzheimer's disease. Neurobiol Aging 1995; 16:409-31.
Wischik C M, Edwards P C, Lai R Y K, Roth M, Harrington C R. Selective inhibition of Alzheimer disease-like tau aggregation by phenothiazines. Proc Natl Acad Sci USA 1996; 93:11213-8.
Wischik C M, Harrington C R, Storey J M D. Tau-aggregation inhibitor therapy for Alzheimer's disease. Biochem Pharmacol 2014; 88:529-39.
Wischik C M, Novak M, Edwards P C, Klug A, Tichelaar W, Crowther R A. Structural characterization of the core of the paired helical filament of Alzheimer disease. Proc Natl Acad Sci USA 1988; 85:4884-8.
Wischik C M, Novak M, Thøgersen H C, et al. Isolation of a fragment of tau derived from the core of the paired helical filament of Alzheimer's disease. Proc Natl Acad Sci USA 1988; 85:4506-10.
Wischik C M, Staff R T, Wischik D J, et al. Tau aggregation inhibitor therapy: an exploratory phase 2 study in mild or moderate Alzheimer's disease. J Alzheimer's Dis 2015; 44:705-20.
Wischik C M, Wischik D J, Storey J M D, Harrington C R. Rationale for tau aggregation inhibitor therapy in Alzheimer's disease and other tauopathies. In: Martinez A, ed. Emerging drugs and targets for Alzheimer's disease Volume 1: Beta-amyloid, tau protein and glucose metabolism. Cambridge: Royal Society of Chemistry; 2010:210-32.
Wilcock, G. K., Gauthier, S., Frisoni, G. B., Jia, J., Hardlund, J. H., Moebius, H. J., Bentham, P., Kook, K. A., Schelter, B. O., Wischik, D. J., Davis, C. S., Staff, R. T., Vuksanovic, V., Ahearn, T., Bracoud, L., Shamsi, K., Marek, K., Seibyl, J., Reidel, G., Storey, J. M. D., Harrington, C. R. & Wischik, C. M. (2018) Potential of low dose leuco-methylthioninium bis(hydromethanesulphonate) (LMTM) monotherapy for treatment of mild Alzheimer's disease: cohort analysis as modified primary outcome in a phase 3 clinical trial. Journal of Alzheimer's Disease 61, 635-657.

### REFERENCES FOR PROTEINS INVOLVED IN DISEASES OF PROTEIN AGGREGATION

Abrahamson, M., Jonsdottir, S., Olafsson, I. & Grubb, A. (1992) Hereditary cystatin C amyloid angiopathy identification of the disease-causing mutation and specific diagnosis by polymerase chain reaction based analysis. Human Genetics 89, 377-380.
Andersen, P. (2006) Amyotrophic lateral sclerosis associated with mutations in the CuZn superoxide dismutase gene. Current Neurology and Neuroscience Reports 6, 37-46.
Arai, T., Hasegawa, M., Nonoka, T., Kametani, F., Yamashita, M., Hosokawa, M., Niizato, K., Tsuchiya, K., Kobayashi, Z., Ikeda, K., Yoshida, M., Onaya, M., Fujishiro, H. & Akiyama, H. (2010) Phosphorylated and cleaved TDP-43 in ALS, FTLD and other neurodegenerative disorders and in cellular models of TDP-43 proteinopathy. Neuropathology 30, 170-181.
Askanas, V., Engel, W. K. & Nogalska, A. (2009) Inclusion body myositis: a degenerative muscle disease associated with intra-muscle fiber multi-protein aggregates, proteasome inhibition, endoplasmic reticulum stress and decreased lysosomal degradation. Brain Pathology 19, 493-506.
Barmada, S. J., Skibinski, G., Korb, E., Rao, E. J., Wu, J. Y. & Finkbeiner, S. (2010) Cytoplasmic mislocalization of TDP-43 is toxic to neurons and enhanced by a mutation associated with familial amyotrophic lateral sclerosis. Journal of Neuroscience 30, 639-649.
Blair, I. P., Williams, K. L., Warraich, S. T., Durnall, J. C., Thoeng, A. D., Manavis, J., Blumbergs, P. C., Vucic, S., Kiernan, M. C. & Nicholson, G. A. (2010) FUS mutations in amyotrophic lateral sclerosis: clinical, pathological, neurophysiological and genetic analysis. Journal of Neurology Neurosurgery and Psychiatry 81, 639-645.
Booth, D. R., Sunde, M., Bellotti, V., Robinson, C. V., Hutchinson, W. L., Fraser, P. E., Hawkins, P. N., Dobson, C. M., Radford, S. E., Blake, C. C. F. & Pepys, M. B. (1997) Instability, unfolding and aggregation of human lysozyme variants underlying amyloid fibrillogenesis. Nature 385, 787-793.
Byrne, S., Walsh, C., Lynch, C., Bede, P., Elamin, M., Kenna, K., McLaughlin, R. & Hardiman, O. (2011) Rate of familial amyotrophic lateral sclerosis: a systematic review and meta-analysis. Journal of Neurology, Neurosurgery & Psychiatry 82, 623-627.
Carrell, R. W. & Gooptu, B. (1998) Conformational changes and disease-serpins, prions and Alzheimer's. Current Opinion in Structural Biology 8, 799-809.
Chen-Plotkin, A. S., Lee, V. M. Y. & Trojanowski, J. Q. (2010) TAR DNA-binding protein 43 in neurodegenerative disease. Nature Reviews Neurology 6, 211-220.
Chiti, F., Webster, P., Taddei, N., Clark, A., Stafani, M., Ramponi, G. & Dobson, C. (1999) Designing conditions for in vitro formation of amyloid protofilaments and fibrils. Proceedings of the National Academy of Sciences, USA 96, 3590-3594.
Cox, L. E., Ferraiuolo, L., Goodall, E. F., Heath, P. R., Higginbottom, A., Mortiboys, H., Hollinger, H. C., Hartley, J. A., Brockington, A., Burness, C. E., Morrison, K. E., Wharton, S. B., Grierson, A. J., Ince, P. G., Kirby, J. & Shaw, P. J. (2010) Mutations in CHMP2B in lower motor neuron predominant amyotrophic lateral sclerosis (ALS). PLOS One 5, e9872.
Czech, C., Tremp, G. & Pradier, L. (2000) Presenilins and Alzheimer's disease: biological functions and pathogenic mechanisms. Progress in Neurobiology 60, 363-384.
Davis, R. L., Shrimpton, A. E., Holohan, P. D., Bradshaw, C., Feiglin, D., Collins, G. H., Sonderegger, P., Kinter, J., Becker, L. M., Lacbawan, F., Krasnewich, D., Muenke, M., Lawrence, D. A., Yerby, M. S., Shaw, C.-M., Gooptu, B., Elliott, P. R., Finch, J. T., Carrell, R. W. & Lomas, D. A. (1999) Familial dementia caused by polymerization of mutant neuroserpin. Nature 401, 376-379.
DiFiglia, M., Sapp, E., Chase, K. O., Davies, S. W., Bates, G. P., Vonsattel, J. P. & Aronin, N. (1997) Aggregation of huntingtin in neuronal intranuclear inclusions and dystrophic neurites in brain. Science 277, 1990-1993.
Dische, F. E., Wernstedt, C., Westermark, G. T., Westermark, P., Pepys, M. B., Rennie, J. A., Gilbey, S. G. & Watkins, P. J. (1988) Insulin as an amyloid-fibril protein at sites of repeated insulin injections in a diabetic patient. Diabetologia 31, 158-161.
Elden, A. C., Kim, H.-J., Hart, M. P., Chen-Plotkin, A. S., Johnson, B. S., Fang, X., Armakola, M., Geser, F., Greene, R., Lu, M. M., Padmanabhan, A., Clay-Falcone, D., Mccluskey, L., Elman, L., Juhr, D., Gruber, P. J., Rub, U., Auburger, G., Trojanowski, J. Q., Lee, V. M. Y., Van Deerlin, V. M., Bonini, N. M. & Gitler, A. D. (2010) Ataxin-2 intermediate-length polyglutamine expansions are associated with increased risk for ALS. Nature 466, 1069-1075.
Finsterer, J (2009) Mitochondrial disorders, cognitive impairment and dementia. J. Neurol. Sci. 283:143-148
Gasset, M., Bladwin, M. A., Lloyd, D. H., abriel, J.-M., Holtzman, D. M., Cohen, F. E., Fletterick, R. & Prusiner, S. B. (1992) Predicted a-helical region of the prion protein when synthesized as peptides form amyloid. Proceedings of the National Academy of Sciences, USA 89, 10940-10944.
Gendron, T. F., Josephs, K. A. & Petrucelli, L. (2010) Review: Transactive response DNA-binding protein 43 (TDP-43): mechanisms of neurodegeneration. Neuropathology and Applied Neurobiology 36, 97-112.
Geser, F., Lee, V. M.-Y. & Trojanowski, J. Q. (2010) Amyotrophic lateral sclerosis and frontotemporal lobar degeneration: A spectrum of TDP-43 proteinopathies. Neuropathology 30, 103-112.
Gitcho, M. A., Baloh, R. H., Chakraverty, S., Mayo, K., Norton, J. B., Levitch, D., Hatanpaa, K. J., White, C. L., III, Bigio. E. H., Caselli, R., Baker, M., Al-Lozi, M. T., Morris, J. C., Pestronk, A., Rademakers, R., Goate, A. M. & Cairns, N. J. (2008) TDP-43 A315T mutation in familial motor neuron disease. Annals of Neurology 63, 535-538.
Glenner, G. G. & Wong, C. W. (1984) Alzheimer's disease: initial report of the purification and characterisation of a novel cerebrovascular amyloid protein. Biochemical and Biophysical Research Communications 120, 885-890.
Goate, A., Chartier-Harlin, M.-C., Mullan, M., Brown, J., Crawford, F., Fidani, L., Giuffra, L., Haynes, A., Irving, N., James, L., Mant, R., Newton, P., Rooke, K., Roques, P., Talbot, C., Pericak-Vance, M., Roses, A., Williamson, R., Rossor, M., Owen, M. & Hardy, J. (1991) Segregation of a missense mutation in the amyloid precursor protein gene with familial Alzheimer's disease. Nature 349, 704-706.
Gorevic, P. D., Casey, T. T., Stone, W. J., DiRaimondo, C. R., Prelli, F. C. & Frangione, B. (1985) b-2 Microglobulin is an amyloidogenic protein in man. Journal of Clinical Investigation 76, 2425-2429.
Gustavsson, A., Engstrom, U. & Westermark, P. (1991) Normal transthyretin and synthetic transthyretin fragments form amyloid-like fibrils in vitro. Biochemical and Biophysical Research Communications 175, 1159-1164.
Higashi, S., Tsuchiya, Y., Araki, T., Wada, K. & Kabuta, T. (2010) TDP-43 physically interacts with amyotrophic lateral sclerosis-linked mutant CuZn superoxide dismutase. Neurochemistry International 57, 906-913.
Hutton, M., Lendon, C., Rizzu, P., Baker, M., Froelich, S., Houlden, H., Pickering-Brown, S., Chakraverty, S., Isaacs, A., Grover, A., Hackett, J., Adamson, J., Lincoln, S., Dickson, D., Davies, P., Petersen, R. C., Stevens, M., de Graaf, E., Wauters, E., van Baren, J., Hillebrand, M., Joosse, M., Kwon, J. M., Nowotny, P., Che, L. K., Norton, J., Morris, J. C., Reed, L. A., Trojanowski, J. Q., Basun, H., Lannfelt, L., Neystat, M., Fahn, S., Dark, F., Tannenberg, T., Dodd, P. R., Hayward, N., Kwok, J. B. J., Schofield, P. R., Andreadis, A., Snowden, J., Craufurd, D., Neary, D., Owen, F., Oostra, B. A., Hardy, J., Goate, A., van Swieten, J., Mann, D., Lynch, T. & Heutink, P. (1998) Association of missense and 5′-splice-site mutations in tau with the inherited dementia FTDP-17. Nature 393, 702-705.
Igaz, L. M., Kwong, L. K., Chen-Plotkin, A., Winton, M. J., Unger, T. L., Xu, Y., Neumann, M., Trojanowski, J. Q. & Lee, V. M. Y. (2009) Expression of TDP-43 C-terminal fragments in vitro recapitulates pathological features of TDP-43 proteinopathies. Journal of Biological Chemistry 284, 8516-8524.
Jinwal, U K, Miyata, Y, Koren, J, III, Jones, J R, Trotter, J H et al. (2009) Chemical manipulation of Hsp70 ATPase activity regulates tau stability. J. Neurosci. 29:12079-12088
Johansson, B., Wernstedt, C. & Westermark, P. (1987) Atrial natriuretic peptide deposited as atrial amyloid fibrils. Biochemical and Biophysical Research Communications 148, 1087-1092.
Johnson, B. S., McCaffery, J. M., Lindquist, S. & Gitler, A. D. (2008) A yeast TDP-43 proteinopathy model: Exploring the molecular determinants of TDP-43 aggregation and cellular toxicity. Proceedings of the National Academy of Sciences 105, 6439-6444.
Johnson, B. S., Snead, D., Lee, J. J., McCaffery, J. M., Shorter, J. & Gitler, A. D. (2009) TDP-43 is intrinsically aggregation-prone, and amyotrophic lateral sclerosis-linked mutations accelerate aggregation and increase toxicity. Journal of Biological Chemistry 284, 20329-20339.
Johnson, J. O., Mandrioli, J., Benatar, M., Abramzon, Y., Van Deerlin, V. M., Trojanowski, J. Q., Gibbs, J. R., Brunetti, M., Gronka, S., Wuu, J., Ding, J., Mccluskey, L., Martinez-Lage, M., Falcone, D., Hernandez, D. G., Arepalli, S., Chong, S., Schymick, J. C., Rothstein, J., Landi, F., Wang, Y.-D., Calvo, A., Mora, G., Sabatelli, M., Monsurrò, M. R., Battistini, S., Salvi, F., Spataro, R., Sola, P., Borghero, G., Galassi, G., Scholz, S. W., Taylor, J. P., Restagno, G., Chiò, A. & Traynor, B. J. (2010) Exome sequencing reveals VCP mutations as a cause of familial ALS. Neuron 68, 857-864.
Kabashi, E., Lin, L., Tradewell, M. L., Dion, P. A., Bercier, V., Bourgouin, P., Rochefort, D., Bel Hadj, S., Durham, H. D., Velde, C. V., Rouleau, G. A. & Drapeau, P. (2010) Gain and loss of function of ALS-related mutations of TARDBP (TDP-43) cause motor deficits in vivo. Human Molecular Genetics 19, 671-683.
Kabashi, E., Valdmanis, P. N., Dion, P., Spiegelman, D., McConkey, B. J., Velde, C. V., Bouchard, J.-P., Lacomblez, L., Pochigaeva, K., Salachas, F., Pradat, P.-F., Camu, W., Meininger, V., Dupre, N. & Rouleau, G. A. (2008) TARDBP mutations in individuals with sporadic and familial amyotrophic lateral sclerosis. Nature Genetics 40, 572-574.
Ling, S.-C., Albuquerque, C. P., Han, J. S., Lagier-Tourenne, C., Tokunaga, S., Zhou, H. & Cleveland, D. W. (2010) ALS-associated mutations in TDP-43 increase its stability and promote TDP-43 complexes with FUS/TLS. Proceedings of the National Academy of Sciences 107, 13318-13323.
Lomas, D. A., Evans, D. L., Finch, J. T. & Carrell, R. W. (1992) The mechanism of Z a1-antitrypsin accumulation in the liver. Nature 357, 605-607.
Love, S., Bridges, L. R. & Case, C. P. (1995) Neurofibrillary tangles in Niemann-Pick disease type C. Brain 118, 119-129.
Mackenzie, I. R. A., Bigio, E. H., Ince, P. G., Geser, F., Neumann, M., Cairns, N. J., Kwong, L. K., Forman, M. S., Ravits, J., Stewart, H., Eisen, A., McClusky, L., Kretzschmar, H. A., Monoranu, C. M., Highley, J. R., Kirby, J., Siddique, T., Shaw, P. J., Lee, V. M. Y. & Trojanowski, J. Q. (2007) Pathological TDP-43 distinguishes sporadic amyotrophic lateral sclerosis from amyotrophic lateral sclerosis with SOD1 mutations. Annals of Neurology 61, 427-434.
Mackenzie, I. R. A., Rademakers, R. & Neumann, M. (2010) TDP-43 and FUS in amyotrophic lateral sclerosis and frontotemporal dementia. The Lancet Neurology 9, 995-1007.
Maury, C. P. & Baumann, M. (1990) Isolation and characterization of cardiac amyloid in familial amyloid polyneuropathy type IV (Finnish): relation of the amyloid protein to variant gelsolin. Biochimica et Biophysica Acta 1096, 84-86.
Neary, D., Snowden, J. S., Gustafson, L., Passant, U., Stuss, D., Black, S., Freedman, M., Kertesz, A., Robert, P. H., Albert, M., Boone, K., Miller, B. L., Cummings, J. & Benson, D. F. (1998) Frontotemporal lobar degeneration: a consensus on clinical diagnostic criteria. Neurology 51, 1546-1554.
Neumann, M. (2009) Molecular neuropathology of TDP-43 proteinopathies. International Journal of Molecular Sciences 10, 232-246.
Neumann, M., Sampathu, D. M., Kwong, L. K., Truax, A. C., Micsenyi, M. C., Chou, T. T., Bruce, J., Schuck, T., Grossman, M., Clark, C. M., Mccluskey, L. F., Miller, B. L., Masliah, E., Mackenzie, I. R., Feldman, H., Feiden, W., Kretzschmar, H. A., Trojanowski, J. Q. & Lee, V. M. Y. (2006) Ubiquitinated TDP-43 in frontotemporal lobar degeneration and amyotrophic lateral sclerosis. Science 314, 130-133.
Nonaka, T., Kametani, F., Arai, T., Akiyama, H. & Hasegawa, M. (2009) Truncation and pathogenic mutations facilitate the formation of intracellular aggregates of TDP-43. Human Molecular Genetics 18, 3353-3364.
Ohmi, K., Kudo, L. C., Ryazantsev, S., Zhao, H.-Z., Karsten, S. L. & Neufeld, E. F. (2009) Sanfilippo syndrome type B, a lysosomal storage disease, is also a tauopathy. Proceedings of the National Academy of Sciences 106, 8332-8337.
Orr, H. T. & Zoghbi, H. Y. (2007) Trinucleotide repeat disorders. Annual Review of Neuroscience 30, 575-621.
Paulson, H. L. (1999) Human genetics '99: trinucleotide repeats. American Journal of Human Genetics 64, 339-345.
Pepys, M. B., Hawkins, P. N., Booth, D. R., Vigushin, D. M., Tennent, G. A., Soutar, A. K., Totty, N., Nguyen. O., Blake, C. C. F., Terry, C. J., Feest, T. G., Zalin, A. M. & Hsuan, J. J. (1993) Human lysozyme gene mutations cause hereditary systemic amyloidosis. Nature 362, 553-557.
Polymeropoulos, M. H., Lavedan, C., Leroy, E., Ide, S. E., Dehejia, A., Dutra, A., Pike, B., Root, H., Rubenstein, J., Boyer, R., Stenroos, E. S., Chandrasekharappa, S., Athanassiadou, A., Papaetropoulos, T., Johnson, W. G., Lazzarini, A. M., Duvoisin, R. C., Di lorio, G., Golbe, L. I. & Nussbaum, R. L. (1997) Mutation in the α-synuclein gene identified in families with Parkinson's disease. Science 276, 2045-2047.
Prusiner, S. B., Scott, M. R., DeArmond, S. J. & Cohen, F. E. (1998) Prion protein biology. Cell 93, 337-348.
Seetharaman, S. V., Prudencio, M., Karch, C., Holloway, S. P., Borchelt, D. R. & Hart, P. J. (2009) Immature copper-zinc superoxide dismutase and familial amyotrophic lateral sclerosis. Experimental Biology and Medicine 234, 1140-1154.
Seilhean, D., Cazeneuve, C., Thuries, V., Russaouen, O., Millecamps, S., Salachas, F., Meininger, V., LeGuern, E. & Duyckaerts, C. (2009) Accumulation of TDP-43 and a-actin in an amyotrophic lateral sclerosis patient with the K171 ANG mutation Acta Neuropathologica 118, 561-573.
Shibata, N., Hirano, A., Kobayashi, M., Siddique, T., Deng, H. X., Hung, W. Y., Kato, T. & Asayama, K. (1996) Intense superoxide dismutase-1 immunoreactivity in intracytoplasmic hyaline inclusions of familial amyotrophic lateral sclerosis with posterior column involvement. Journal of Neuropathology and Experimental Neurology 55, 481-490.
Sletten, K., Westermark, P. & Natvig, J. B. (1976) Characterization of amyloid fibril proteins from medullary carcinoma of the thyroid. Journal of Experimental Medicine 143, 993-998.
Spillantini, M. G., Crowther, R. A., Jakes, R., Hasegawa, M. & Goedert, M. (1998) a-Synuclein in filamentous inclusions of Lewy bodies from Parkinson's disease and dementia with Lewy bodies. Proceedings of the National Academy of Sciences, USA 95, 6469-6473.
Sreedharan, J., Blair, I. P., Tripathi, V. B., Hu, X., Vance, C., Rogelj. B., Ackerley, S., Durnall, J. C., Williams, K. L., Buratti, E., Baralle, F., de Belleroche, J., Mitchell, J. D., Leigh, P. N., Al-Chalabi, A., Miller, C. C., Nicholson, G. & Shaw, C. E. (2008) TDP-43 mutations in familial and sporadic amyotrophic lateral sclerosis. Science 319, 1668-1672.
Uemichi, T., Liuepnicks, J. j. & Benson, M. D. (1994) Hereditary renal amyloidosis with a novel variant fibrinogen. Journal of Clinical Investigation 93, 731-736.
van Bebber, F., Paquet, D., Hruscha, A., Schmid, B. & Haass, C. (2010) Methylene blue fails to inhibit Tau and polyglutamine protein dependent toxicity in zebrafish. Neurobiology of Disease 39, 265-271.
Vance, C., Rogelj, B., Hortobagyi, T., De Vos, K. J., Nishimura, A. L., Sreedharan, J., Hu, X., Smith, B., Ruddy, D., Wright, P., Ganesalingam, J., Williams, K. L., Tripathi, V., Al-Saraj, S., Al-Chalabi, A., Leigh, P. N., Blair, I. P., Nicholson, G., de Belleroche, J., Gallo, J.-M., Miller, C. C. & Shaw, C. E. (2009) Mutations in FUS, an RNA processing protein, cause familial amyotrophic lateral sclerosis type 6. Science 323, 1208-1211.
Westermark, P., Engstrom, U., Johnson, K. H., Westermark, G. T. & Betsholtz, C. (1990) Islet amyloid polypeptide: pinpointing amino acid residues linked to amyloid fibril formation. Proceedings of the National Academy of Sciences, USA 87, 5036-5040.
Westermark, P., Johnson, K. H. & Pitkanen, P. (1985) Systemic amyloidosis: A review with emphasis on pathogenesis. Applied Physiology 3, 55-68.
Westermark, P., Johnson, K. H., O'Brien, T. D. & Betsholtz, C. (1992) Islet amyloid polypeptide-a novel controversy in diabetes research. Diabetologia 35, 297-303.
Wijesekera, L. & Leigh, P. N. (2009) Amyotrophic lateral sclerosis. Orphanet Journal of Rare Diseases 4, 3.
Wischik, C. M., Novak, M., Thøgersen, H. C., Edwards, P. C., Runswick, M. J., Jakes, R., Walker, J. E., Milstein, C., M., R. & Klug, A. (1988) Isolation of a fragment of tau derived from the core of the paired helical filament of Alzheimer's disease. Proceedings of the National Academy of Sciences, USA 85, 4506-4510.
Yamashita, M., Nonaka, T., Arai, T., Kametani, F., Buchman, V. L., Ninkina, N., Bachurin, S. O., Akiyama, H., Goedert, M. & Hasegawa, M. (2009) Methylene blue and dimebon inhibit aggregation of TDP-43 in cellular models. FEBS Letters 583, 2419-2424.
Zhang, Y.-J., Xu, Y.-F., Cook, C., Gendron, T. F., Roettges, P., Link, C. D., Lin, W.-L., Tong, J., Castanedes-Casey, M., Ash, P., Gass, J., Rangachari, V., Buratti, E., Baralle, F., Golde, T. E., Dickson, D. W. & Petrucelli, L. (2009) Aberrant cleavage of TDP-43 enhances aggregation and cellular toxicity. Proceedings of the National Academy of Sciences 106, 7607-7612.

### REFERENCES FOR INVENTION DISCLOSURE AND EXAMPLES

Alhola P, Polo-Kantola P. Sleep deprivation: Impact on cognitive performance. Neuropsychiatr Dis Treat. 2007; 3:553-67.
Eugene, A. R., and Masiak, J. (2015). The Neuroprotective Aspects of Sleep. MEDtube Sci 3, 35-40.
Wischik C M, Bentham P, Gauthier S, Miller S, Kook K, Schelter B O; “Oral Tau aggregation inhibitor for Alzheimer's disease: design, progress and basis for selection of the 16 mg/day dose in a Phase 3, randomized, placebo-controlled trial of hydromethylthionine mesylate”; Journal of Prevention of Alzheimer's Disease; 2022; 9:780-790.

1. A method of treatment of a neurodegenerative disease in a subject, which method comprises orally administering to said subject a methylthioninium (MT) containing compound,
wherein said administration is at a dosage frequency of less than once daily;
and wherein said administration provides an amount of MT to the subject that corresponds to an average of between 0.05 mg and 30 mg MT per day, preferably between 0.1 mg and 20 mg MT per day.

2. A method as claimed in claim 1, wherein the dosage frequency is ≤½, ≤⅓, or ≤¼, wherein the dosage frequency is defined as the total number of doses in a given treatment period, divided by the number of days in said treatment period.

3. A method as claimed in claim 1 or claim 2, wherein doses of the MT-compound are not administered on consecutive days.

4. A method as claimed in any one of the preceding claims, wherein dosage is at regular intervals.

5. A method as claimed in any one of the preceding claims, wherein dosage is irregular or intermittent.

6. A method as claimed in claim 4, wherein the dosage frequency is every two days, every three days, every four days, every five days, every six days.

7. A method as claimed in claim 4, wherein the dosage frequency is weekly, twice weekly, or thrice weekly, on fixed day(s) of each week.

8. A method as claimed in claim 5, wherein the dosage frequency is weekly, twice weekly, or thrice weekly, on variable or random days in each week.

9. A method as claimed in claim 8, wherein the dosage frequency is twice weekly, on a varying schedule each week.

10. A method as claimed in any one of the preceding claims, wherein administration provides an amount of MT to the subject that corresponds to an average amount per day of from around any of 0.05, 0.1, 0.2, 0.3, 0.4, 0.5, 1, 1.5, and 2 mg to around any of 2.5, 3, 5, 6, 7, 8, 9, 10, 11, 12, 13, 14, 15, 16, 17, 18, 19, 20, 25 and 30 mg.

11. A method as claimed in claim 10, wherein the average amount of MT per day is from 0.05 to 30 mg, 0.05 to 20 mg, 0.05 to 10 mg, 0.05 to 5 mg, 0.05 to 3 mg, 0.1 to 30 mg, 0.1 to 20 mg, 0.1 to 10 mg, 0.1 to 5 mg, 0.1 to 3 mg, 0.2 to 20 mg; 0.2 to 10 mg; from 0.2 to 5 mg; from 0.2 to 3 mg; from 0.3 to 10 mg; from 0.3 to 5 mg; from 0.3 to 3 mg; from 0.4 to 10 mg; from 0.4 to 5 mg; from 0.4 to 3 mg; from 0.5 to 10 mg; from 0.5 to 5 mg; or from 0.5 to 3 mg.

12. A method as claimed in any one of the preceding claims, wherein the amount of MT provided by each dose of the methylthioninium (MT) containing compound is from around any of 0.1, 0.2, 0.4, 0.5, 0.6, 0.8, 1.0, 1.2, and 1.4 mg MT to around any of 5, 10, 20, 40, 50, 60, 70, 80, 100, 120 and 140 mg MT.

13. A method as claimed in any one of claims 1 to 5, wherein:
the dosage frequency is ≤½ and the amount of MT provided in each dose is between 0.4 and 40 mg; or
the dosage frequency is every other day and the amount of MT provided in each dose is between 0.4 and 40 mg; or
the dosage frequency is ≤⅓ and the amount of MT provided in each dose is between 0.6 and 60 mg; or
the dosage frequency is every 3 days and the amount of MT provided in each dose is between 0.6 and 60 mg; or
the dosage frequency is ≤¼ and the amount of MT provided in each dose is between 0.8 and 80 mg;
the dosage frequency is every 4 days and the amount of MT provided in each dose is between 0.8 and 80 mg; or
the dosage frequency is every 5 days and the amount of MT provided in each dose is between 1 and 100 mg; or
the dosage frequency is every 6 days and the amount of MT provided in each dose is between 1.2 and 120 mg; or
the dosage frequency is weekly and the amount of MT provided in each dose is between 1.4 and 140 mg; or
the dosage frequency is twice weekly and the amount of MT provided in each dose is between 0.7 and 70 mg; or
the dosage frequency is thrice weekly and the amount of MT provided in each dose is between 0.5 and 50 mg.

14. A method of treatment of a neurodegenerative disease in a subject, which method comprises orally administering to said subject a methylthioninium (MT) containing compound,
wherein said administration provides an amount of MT to the subject that corresponds to less than 0.5 mg MT per day.

15. A method as claimed in claim 14, wherein the amount of MT per day is from 0.05 to less than 0.5 mg.

16. A method as claimed in claim 14, wherein administration provides an amount of MT to the subject that corresponds to an average amount per day of from 0.05 to any of 0.2, 0.3, 0.4, 0.45, 0.49, and 0.495 mg.

17. A method as claimed in any of the preceding claims, wherein administration provides an amount of MT to the subject which results in a plasma MT level of at least 0.10 ng/ml, as measured at 12 months of treatment.

18. A method as claimed in any of the preceding claims, wherein administration provides an amount of MT to the subject which results in a plasma MT level of at least 0.23 ng/ml, as measured at 12 months of treatment.

19. A method as claimed in any one of claims 1 to 18 wherein the compound is a salt of either: 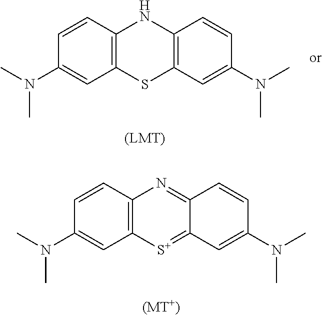
or a hydrate or solvate thereof.

20. A method as claimed in claim 19 wherein a mixture of LMT and MT⁺ containing compounds are administered.

21. A method as claimed in claim 19 wherein the compound is an LMT compound.

22. A method as claimed in claim 21 wherein the compound is an LMTX compound of the following formula: 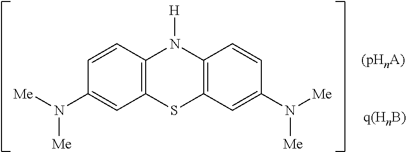
wherein each of HₙA and HₙB (where present) are protic acids which may be the same or different,
and wherein p=1 or 2; q=0 or 1; n=1 or 2; (p+q)×n=2.

23. A method as claimed in claim 22 wherein the compound has the following formula, where HA and HB are different mono-protic acids:

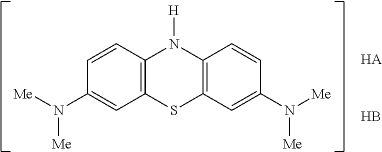

24. A method as claimed in claim 22 wherein the compound has the following formula: 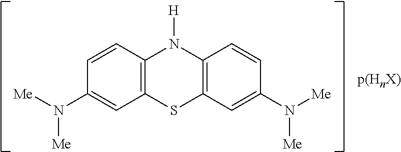
wherein each of HₙX is a protic acid.

25. A method as claimed in claim 22 wherein the compound has the following formula and H₂A is a di-protic acid:

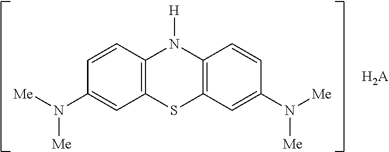

26. A method as claimed in claim 22 wherein the compound has the following formula and is a bis-monoprotic acid:

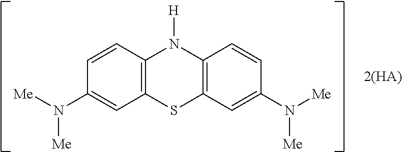

27. A method as claimed in any one of claims 22 to 26 wherein the or each protic acid is an inorganic acid.

28. A method as claimed in claim 27 wherein each protic acid is a hydrohalide acid.

29. A method as claimed in claim 27 wherein the or each protic acid is selected from HCl; HBr; HNO₃; H₂SO₄.

30. A method as claimed in any one of claims 22 to 26 wherein the or each protic acid is an organic acid.

31. A method as claimed in claim 30 wherein the or each protic acid is selected from H₂CO₃; CH₃COOH; methanesulfonic acid, 1,2-ethanedisulfonic acid, ethansulfonic acid, naphthalenedisulfonic acid, p-toluenesulfonic acid.

32. A method as claimed in claim 31 wherein the compound is LMTM:

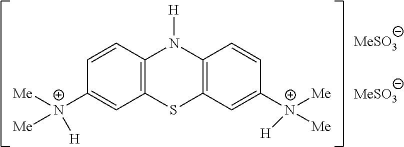

33. A method as claimed in claim 32 wherein administration provides an average amount of LMTM per day of about 0.08 to 50 mg, preferably about 0.17 to 33 mg.

34. A method as claimed in claim 32 wherein the amount of LMTM in each dose is about 27 mg; about 14 mg; about 7 mg, or about 2 mg.

35. A method as claimed in claim 31 wherein the compound is selected from the list consisting of:

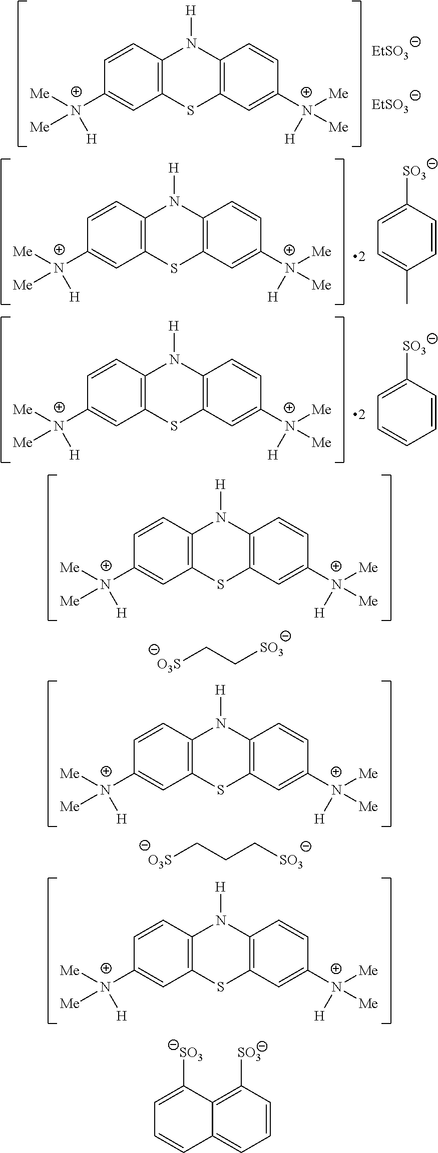

36. A method as claimed in claim 19 wherein the compound is an MT⁺ salt having the formula or being a hydrate, solvate, or mixed salt thereof: 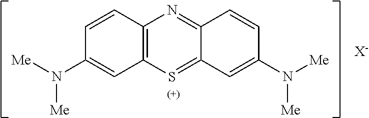
where X⁻ is an anionic counter ion.

37. A method as claimed in claim 36 wherein the compound is MTC.

38. A method as claimed in claim 37 wherein the compound is MTC polymorph A pentahydrate.

39. A method as claimed in any one of claims 36 to 38 wherein the compound is characterised by a purity of greater than 98%.

40. A method as claimed in any one of claims 36 to 39, wherein the compound is characterised by a purity of greater than 98% and one or more of the following:
(i) less than 1% Azure B as impurity;
(ii) less than 0.13% MVB (Methylene Violet Bemstein) as impurity;
(iii) less than 0.15% Azure A as impurity;
(iv) less than 0.15% Azure C as impurity; or
(v) an elementals purity better than less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

41. A method as claimed in any one of claims 36 to 40, wherein the compound is characterised by a purity of greater than 98% and less than 1% Azure B as impurity.

42. A method as claimed in any one of claims 36 to 41, wherein the compound is characterised by a purity of greater than 98% and an elementals purity better than less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

43. A method as claimed in any one of claims 36 to 42, wherein the compound is characterised by:
(i) at least 98% purity
(i) less than 1% Azure B as impurity; and
(ii) an elementals purity better than the European Pharmacopeia limits of less than 100 μg/g Aluminium (Al); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

44. A method as claimed in any one of claims 37 to 43 wherein administration provides an average amount of MTC·5H₂O per day of about 0.07 to 43 mg, preferably about 0.14 to 29 mg.

45. A method as claimed in any one of claims 37 to 44 wherein each dose comprises about 16 mg; about 8 mg; about 4 mg, or about 2 mg of MTC·5H₂O.

46. A method as claimed in any one of claims 32 to 40 wherein each dose comprises about 4 mg MT and the dosage frequency is twice weekly.

47. A method as claimed in claim 37 wherein the compound is selected from: MTC·0.5ZnCl₂; MTI; MTI·HI; MT·NO₃.

48. A method as claimed in any one of claims 37 to 47 wherein the MT⁺ salt is formulated with a reducing agent which is optionally ascorbate, and then optionally lyophilized.

49. A method as claimed in any one of claims 1 to 48 wherein the duration of treatment with the MT compound is at least 7, 8, 9, 10, 11, 12 months, or longer; optionally at least 1, 2, 3, 4, 5 years, or longer.

50. A method as claimed in any one of claims 1 to 49 wherein the subject is a human who has been diagnosed as having said cognitive or CNS disorder, or wherein said method comprises making said diagnosis.

51. A method of prophylactic treatment of a neurodegenerative disorder of protein aggregation in a subject,
which method comprises orally administering to said patient a methylthioninium (MT) containing compound,
wherein said administration is as defined in any one of claims 1 to 50.

52. A method as claimed in claim 51 wherein the subject is a human who has been assessed as being susceptible to the cognitive or CNS disorder, optionally based on familial or genetic or other data.

53. A method as claimed in any one of claims 1 to 52 wherein the disorder is a tauopathy.

54. A method as claimed in any one of claims 1 to 53 wherein the disorder is selected from the list consisting of: Pick's disease, progressive supranuclear palsy, frontotemporal dementia, FTD with parkinsonism linked to chromosome 17, frontotemporal lobar degeneration syndromes; disinhibition-dementia-parkinsonism-amyotrophy complex, pallido-ponto-nigral degeneration, Guam-ALS syndrome, pallido nigro luysian degeneration, cortico-basal degeneration, dementia with argyrophilic grains, dementia pugilistica or chronic traumatic encephalopathy, Down's syndrome, subacute sclerosing panencephalitis, mild cognitive impairment, Niemann-Pick disease, type C, Sanfilippo syndrome type B, or a myotonic dystrophy DM1 or DM2.

55. A method as claimed in any one of claims 1 to 54 wherein the disorder is Alzheimer's disease.

56. A method as claimed in any one of claims 1 to 54 wherein the disorder is mild cognitive impairment.

57. A method as claimed in any one of claims 1 to 54 wherein the disorder is a polyglutamine disorder, such as Huntington's disease, spinal bulbar muscular atrophy, dentatorubropallidoluysian atrophy or spinocerebellar ataxias; wherein the disorder is a TDP-43 proteinopathy, such as FTLD-TDP; wherein the disorder is a synucleinopathy, such as Parkinson's disease, dementia with Lewy bodies or multiple system atrophy; wherein the disorder is hereditary cerebral angiopathy; wherein the disorder is amyotrophic lateral sclerosis; or wherein the disorder is familial encephalopathy with neuronal inclusion bodies.

58. A pharmaceutical composition comprising an MT compound as defined in any one of claims 1 to 47, and a pharmaceutically acceptable carrier or diluent, optionally in the form of a dosage unit, wherein the amount of MT in the composition or unit is less than 0.5 mg.

59. A pharmaceutical composition according to claim 58, wherein the amount of MT in the composition or unit is from any one of 0.05 mg, 0.1 mg, 0.2 mg, and 0.3 mg to any one of 0.45, 0.46, 0.47, 0.48, 0.48, and about 0.5 mg.

60. A composition as claimed in any one of claims 58 to 59 which is a tablet or capsule.

61. A composition as claimed in any one of claims 58 to 60 which is an immediate release composition.

62. A container comprising:
(i) a plurality of dosage units each of which is a composition as claimed in any one of claims 58 to 61;
(ii) a label and/or instructions for their use according to a method as defined in any one of claims 1 to 57.

63. A container as claimed in claim 62, wherein the container comprises dosage units, and the dosage units are present in a blister pack which is substantially moisture-impervious.

64. A container as claimed in claim 62 or claim 63 wherein the label or instructions provide information regarding the disorder for which the composition is intended.

65. A container as claimed in any one of claims 62 to 64 wherein the label or instructions provide information regarding the maximum permitted dosage and/or the permitted frequency of dosage of the dosage units.

66. A container as claimed in any one of claims 62 to 64 wherein the label or instructions provide information regarding the suggested duration of the treatment.

67. An MT-containing compound or composition as described in any one of claims 1 to 61, for use in a method of treatment as defined in any one of claims 1 to 57.

68. Use of an MT-containing compound or composition as described in any one of claims 1 to 61, in the manufacture of a medicament for use a method of treatment as defined in any one of claims 1 to 57.
